SEQUENCE LISTING (4 sequences)

SEQ ID NO: 1 - protein, 4 aa, Artificial
  ER retention signal
KDEL

SEQ ID NO: 2 - protein, 7 aa, Simian virus 40
PKKKRKV

SEQ ID NO: 3 - DNA, 20 bp, Artificial
  Synthetic oligonucleotide primer
ggaaagccacgttgtgtctc

SEQ ID NO: 4 - DNA, 21 bp, Artificial
  Synthetic oligonucleotide primer
cgatttattcaacaaagccgc

(19) United States
(12) Patent Application Publication
(10) Pub. No.: US 2006/0286574 A1
(43) Pub. Date: Dec. 21, 2006

(54) Compositions and methods for enhancing drug sensitivity and treating drug resistant infections and diseases

(75) Inventors: Floyd Romesberg, La Jolla, CA (US); Ryan Cirz, San Diego, CA (US); Philip A. Patten, Portola Valley, CA (US)

Correspondence Address: QUINE INTELLECTUAL PROPERTY LAW GROUP, P.C., P O BOX 458, ALAMEDA, CA, 94501, US

(73) Assignees: The Scripps Research Institute & Achaogen, Inc., La Jolla, CA (US); Achaogen Inc., South San Francisco, CA (US)

(21) Appl. No.: 11/399,294

(22) Filed: Apr. 5, 2006

Related U.S. Application Data
(60) Provisional application No. 60/668,737, filed on Apr. 5, 2005.

(51) Int. Cl. C12Q 1/68; A61K 31/496; A61K 31/4709; C40B 30/06

(52) U.S. Cl. 435/6; 514/253.08; 514/312

(57) ABSTRACT

The present invention provides compositions useful in treating drug-resistant microorganisms and cells, as well as related methods of identifying and using such compositions. In addition, the present invention includes compositions useful in enhancing the sensitivity of both drug-resistant and drug-sensitive microorganisms and cells to microbicidal and cytotoxic agents, including antibiotics and chemotherapeutic drugs. Methods of identifying these compositions, as well as methods of using these agents in treating both drug-resistant and drug-sensitive diseases and conditions are further provided.

### CROSS-REFERENCE TO RELATED APPLICATION

This application claims the benefit under 35 U.S.C. § 119(e) of U.S. Provisional Patent Application No. 60/668,737, filed Apr. 5, 2005, where this provisional application is incorporated herein by reference in its entirety.

### FIELD OF THE INVENTION

The present invention is directed to compositions and methods useful in the treatment of drug-resistant microorganisms and cells. These compositions and methods are also useful in increasing the sensitivity of microorganisms and cells to antimicrobial and cytotoxic agents and, therefore, in the treatment of both drug-sensitive and drug-resistant infections and diseases, including, e.g., tumors.

### BACKGROUND OF THE INVENTION

Drug resistance is an ever-increasing problem in modern medicine that hampers the treatment of conditions as diverse as bacterial infections, viral infections, protozoan infections, fungal infections, and cancer. For example, the worldwide emergence of antibiotic-resistant bacteria threatens to undo the dramatic advances in human health that followed the discovery of these drugs. Clonal spread of drug resistant bacteria, and horizontal transfer of resistance factors among bacteria has resulted in a dramatic increase in the frequency of drug resistant bacteria over the last two decades.

Antibiotic drug resistance is especially acute with tuberculosis, which infects one-third of all humans, most of whom live in the developing world. The health-care establishment is countering this challenge by attempting to create new antibiotics and by limiting the use of those already available. However, this approach has not yet produced the desired effect, as the prevalence of resistant strains continues to increase.

The evolution of resistance is expected to be especially problematic in the event of a bioterrorist attack, due to the potential numbers of infected individuals, the likely continued inappropriate use of antibiotics with a consequent evolution of resistance during therapy, and the continued transmission of a resistant strain. An even more alarming possibility is the release of a drug-resistant deadly microorganism, such as a Bacillus anthracis or Yersinia pestis strain that has been engineered to be resistant to all available antibiotics. Such deadly strains can be constructed, and are in fact thought to exist already. It is, therefore, absolutely essential that effective countermeasures to these threats be developed.

Drug resistance is also a problem with other microorganisms, including viruses and protozoa, such as the human immunodeficiency virus (HIV). In fact, HIV drug resistance is rapidly becoming an epidemic. One study of HIV infected patients between 1996 and 1999 showed that about 78% of patients harbored viruses that were resistant to at least one class of drugs, 51% had viruses that were resistant to two classes of drugs, and 18% had viruses that were resistant to three classes of drugs. Thus, HIV drug therapies must constantly evolve to keep pace with the evolution of resistance. Similarly, in recent years, drug resistance of Plasmodium spp. has become one of the most important problems in malaria control. Resistance in vivo has been reported to all anti-malaria drugs except artemisinin and its derivatives. Such continued increase in drug resistance necessitates the use of drugs that are more expensive and that may have dangerous side effects.

Drug resistance is also a problem during cancer therapy. It is estimated that nearly half of all cancer patients are cured, mostly by a combination of surgery, radiotherapy and/or chemotherapy. However, some cancers can only be treated by chemotherapy, and in those cases, only one in five patients survive long-term. It is believed that the overriding reason for this poor result is drug resistance, wherein the tumors are either innately resistant to the drugs available, or else are initially sensitive but evolve resistance during treatment and eventually re-grow (Allen J D, et al. Cancer Research 62, 2294-2299 (2002)).

Clearly, there is a great need for compounds that have cytostatic and cidal properties against drug-resistant microorganisms and cells, for compounds that sensitize microorganisms and cells to existing drugs, compounds that inhibit mutational processes that lead to drug resistance, compounds that prevent horizontal spread of resistance factors, and methods for identifying and using such compounds to treat and prevent drug resistant diseases and conditions, as well as to enhance the efficacy of such compounds in treating and preventing both drug sensitive and drug resistant infections and diseases.

### SUMMARY OF THE INVENTION

The present invention establishes that DNA repair and replication pathways play a fundamental role in the establishment and maintenance of drug resistance, as well as drug sensitivity of microorganisms and cells. The invention provides a variety of methods and related compositions useful in treating drug resistance and increasing drug sensitivity of microorganisms and cells. In general, the methods and compositions of the invention are related to the identification of inhibitors of a DNA repair, replication pathway, or recombination (“inhibitors”), compositions comprising an inhibitor and a antimicrobial or cytotoxic compound, and methods of using inhibitors to treat drug-resistance microorganisms and cells, enhance drug sensitivity of microorganisms and cells, and treat microbial infections and cancers. Inhibitors of the present invention generally have the ability to enhance the sensitivity of a microorganism or cell to an antimicrobial or cytotoxic agent or are cytotoxic for a drug-resistant microoganism or cell.

Inhibitors of the invention target a DNA repair, replication, or recombination pathway (or polypeptide associated with such a pathway) of any microorganism or cell, including mammalian cells. Specific pathways include double-stranded DNA break repair and stalled replication fork rescue or repair pathways. Examples of double-stranded DNA break repair pathways include homologous recombination, such as RecBCD-mediated and RecFOR-mediated homologous recombination, as well as non-homologous recombination or end joining. Examples of stalled replication fork rescue and repair pathways include recombination-dependent fork repair, replication restart, and primosome reassembly as well as homologous or non-homologous recombination. Accordingly, specific polypeptide targets include any polypeptide associated with a DNA repair or replication pathway, including, e.g., RecA, RecB, PriA, DNA-PK, Ku70, and Ku86. Inhibitors may be any type of molecule capable of inhibiting the activity or expression of a polypeptide associated with DNA repair or replication, such as small molecules, peptides or mimetics thereof, polynucleotides (e.g., antisense and RNAi), and polypeptides (e.g., antibodies). Specific activities that may be inhibited include polymerase, endonuclease, exonuclease, helicase activities as well as chi sequence recognition and RecA functions, including filamentation on DNA or helicase/ATPase activities.

Methods of identifying inhibitors can be based upon their ability to either bind or inhibit an activity of a polypeptide associated with DNA repair or replication. In general, methods of identifying inhibitors include: (a) screening one or more candidate agents for their ability to bind or inhibit an activity of a polypeptide associated with DNA repair, replication, or recombination; (b) identifying a candidate agent identified in step (a) that sensitizes a microorganism or cell to an antimicrobial or cytotoxic compound or kills a drug resistant microorganism or cell. Methods may further include: (c) producing a derivative or analog of the agent identified in step (b); and (d) determining whether said derivative or analog enhances the sensitivity of the microorganism or cell to the antimicrobial or cytotoxic compound or kills a drug-resistant microorganism or cell. Such methods may be practiced using any of a variety of binding or activity assays, e.g., using isolated polypeptides, cellular extracts, or whole cells.

Methods of enhancing the activity of an antimicrobial or cytotoxic compound, which include administering an antimicrobial or cytotoxic compound in combination with an inhibitor are provided. The inhibitor may be administered before, during, or after administration of the antimicrobial or cytotoxic agent. The inhibitor and agent may be administered to a patient suffering from a microbial infection or a tumor. Furthermore, the invention includes methods of inhibiting the growth or proliferation of a microorganism or cell, comprising: administering an inhibitor to a microorganism or cell. Related methods of the invention include a method of sensitizing a microorganism or cell to an antimicrobial or cytotoxic compound, comprising contacting said microorganism or cell with an inhibitor.

The invention can be used diagnostically and therapeutically in treating a patient infected with a microorganism, by: (a) determining if the microorganism contains a mutation in a gene associated with resistance to an antimicrobial compound; and (b) administering an inhibitor to the patient, if the microorganism contains a mutation in a gene associated with resistance to an antimicrobial compound. In certain aspects, the mutated gene may be either parC or gyrA or homologs thereof.

The invention further includes a method of inhibiting the acquisition of drug resistance by a microorganism, comprising: contacting the microorganism with an inhibitor during treatment with said drug, wherein the inhibitor reduces transfer of a resistance-conferring gene, e.g., via homologous recombination or conjugal transfer.

The invention also includes compositions and kits suitable for carrying out methods of the invention. Such compositions and kits generally include an antimicrobial or cytotoxic compound and an inhibitor. The antimicrobial or cytotoxic compound and inhibitor may be formulated separately or in combination, e.g., as a tablet. In addition, the compositions of the invention, including inhibitors, may be formulated for any known route of administration, including, e.g., parenteral and oral administration.

The invention also includes a process of producing an inhibitor, which generally includes: (a) screening a library of compounds to identify a compound that inhibits an activity of a polypeptide associated with DNA repair, replication or recombination; (b) derivatizing the identified compound; (c) testing the derivatized compound for its ability to inhibit an activity of a polypeptide associated with DNA repair or replication; and (d) producing the derivatized compound.

Another use of an inhibitor is to increase the therapeutic index or reduce systemic toxicity of an antimicrobial or cytotoxic compound, by providing the antimicrobial or cytotoxic compound in combination with an inhibitor.

The methods and compositions of the invention can be directed to any known or yet to be discovered antimicrobial or cytotoxic agent or compound (i.e., drugs), including those that act by causing DNA damage or inhibiting DNA replication, repair or recombination. In addition, the methods and compositions of the invention may be used with drugs for which resistance has already developed or drugs for which resistance has not yet developed. Examples of specific antimicrobial and cytotoxic agents include fluoroquinolones and topoisomerase poisons.

Furthermore, the methods and compositions of the invention can be directed to any type of microorganisms or cell, including, e.g., bacteria, fungi, and eukaryotic cells, including mammalian cells (e.g., tumor cells). Bacteria may be either gram positive or gram negative, and specific bacterial species include, but are not limited to: ciprofloxacin-resistant S. aureus, coagulase-negative Staph, E. faecalis, E. faecium, E. coli, K. oxytoca, K. pneumoniae, M. morganii, P. mirabilis, S. marcescens, Acinetobacter, and P. aeruginosa; levofloxacin-resistant S. pneumoniae, S. pyogenes, S. agalactiae, Viridans group, E. coli, K. oxytoca, K. pneumoniae, M. morganii, P. mirabilis, S. marcenscens, Acinetobacter, and P. aeruginosa; sulfamethoxazole trimethoprim-resistant E. coli, K. oxytoca, K. pneumoniae, M. Morganii, P. mirabilis, S. marcenscens, Acinetobacter, and P. aeruginosa; ampicillin-resistant S. aureus, coagulase-negative staph, E. faecalis, E. faecium, and S. pneumoniae; oxacillin-resistant S. aureus and coagulase-negative staph; penicillin-resistant S. pneumoniae and Virdans group; piperacillin-tazobactam-resistant E. coli, K. oxytoca, K. pneumoniae, M. morganii, P. mirabilis, S. marcescens, Acinetobacter, and P. aeruginosa; cefapine-resistant S. aureus, coagulase-negative staph, S. pneumoniae, E. coli, K. oxytoca, K. pneumoniae, M. morganii, P. mirabilis, S. marcescens, Acinobacter, and P. aeruginosa; cefotaxime-resistant S. aureus, coagulase-negative staph, S. pneumoniae, E. coli, K. oxytoca, K. pneumoniae, M. morganii, P. mirabilis, S. marcenscens, Acinetobacter, and P. aeruginosa; ceftriaxone-resistant S. aureus, coagulase-negative staph, S. pneumoniae, M. morganii, P. mirabilis, S. marcescens, Acinetobacter, and P. aeruginosa; gentamycin-resistant S. aureus, coagulase-negative staph, E. faecalis, E. faecium, E. coli, K. oxytoca, K. pneumoniae, M. morganii, P. mirabilis, S. marcenscens, Acinobacter, and P. aeruginosa; clarithromycin-resistant S. pneumoniae, S. pyogenes, S. agalactiae, and Virdans group; erythromycin-resistant S. pneumoniae, S. pyogenes, and S. agalactiae, and Virdans group; teicoplanin-resistant E. faecium; vancomycin-resistant E. faecalis and E. faecium; and imipenem-resistant Acinobacter and P. aeruginosa.

### BRIEF DESCRIPTION OF THE FIGURES

FIG. 1 provides a diagram of the bacterial response to ciprofloxacin. In the absence of homologous sequences, free double strand breaks (DSBs) are repaired by nuclease and polymerase-dependent illegitimate recombination (IR, Pathway A). In the presence of a suitable homologous sequence and a functional homologous recombination system, free DSBs can be repaired by replication-dependant recombination (RDR, Pathway B). This pathway can also contribute to the repair of replication forks when they encounter the free DSB. Finally, inhibited replication forks are repaired by recombination-dependent replication fork repair (Pathway C).

FIG. 2 is a graph depicting the number of viable cells remaining at the indicated time points (colony forming units on LB) following plating of the various recombination mutants on solid media containing 40 ng/ml ciprofloxacin. Visible colonies were excised from the primary ciprofloxacin containing selective plates prior to determination of viable counts on LB.

FIG. 3 illustrates a stressful lifestyle adaptive mutation (SLAM) assay.

FIG. 4 is a graph depicting the number of viable cells remaining at the indicated time points following plating on solid media containing 40 ng/mL ciprofloxacin of wild-type and mutant strains. (A) recombination mutants that were hypersensitive to ciprofloxacin and (B) recombination mutants with wild-type sensitivity. Values represent the number of cells surviving per day and error bars represent standard deviation from at least three independent determinations. Visible colonies were excised from the primary ciprofloxacin containing selective plates prior to determination of viable counts on LB.

FIG. 5 is a graph depicting the minimum inhibitory concentration (MIC) under permissive and non-permissive conditions of various ciprofloxacin resistant strains derived from SK119. SK119 is a strain of E. coli that bears a temperature sensitive recB allele (Kushner, S. et al. J. Bact. 120:1213-1218, 1974). SK119 indicates the wild-type SK119 strain; 1, 3, 5, 6, and 8 each indicate separate strains of ciprofloxacin resistant mutants that were selected at the permissive temperature, as described in Example 4.

FIG. 6 is a graph depicting the effect of deletion of recB on the ciprofloxacin sensitivity of Kleibsiella Pneumoniae in a murine thigh infection model. The graph shows the log cfu/g of thigh muscle in animals infected with wild type or recB mutant strains of Kleibsiella Pneumoniae at various dosages of ciprofloxacin.

### DETAILED DESCRIPTION OF THE INVENTION

The present invention includes the surprising discovery that drug-resistant microorganisms and cells require DNA repair or replication pathways for survival in the presence of otherwise non-lethal concentrations of fluoroquinolone, and that inhibiting the activity of a polypeptide involved in DNA repair, such as, e.g., the E. coli proteins RecA, RecBC(D), or PriA, or homologs thereof, results in reduced proliferation and/or increased death of drug-resistant cells, in the presence of an antimicrobial or cytotoxic agent or compound. In addition, certain aspects of the present invention are based on the related discovery that inhibiting the activity of a polypeptide involved in DNA repair and replication results in increased sensitivity of both drug-resistant and drug-sensitive cells to antimicrobial and cytotoxic agents.

In light of these discoveries, the present invention provides compounds and compositions that inhibit one or more DNA repair or replication pathways, as well as related methods of identifying and using such compositions, e.g., in the treatment of microbial infections and tumors. The compounds and compositions of the present invention inhibit the activity or expression of one or more polypeptide components of a DNA repair or replication pathway in a microorganism or other cell, including a mammalian cell, either directly or indirectly. By inhibiting a DNA repair or replication pathway, compounds of the present invention sensitize cells and microorganism to an antimicrobial or cytotoxic agent. In addition, compounds of the present invention are effective in killing or reducing growth of drug-resistant microorganisms and cells that require a DNA repair or replication pathway for survival.

A number of antimicrobial and cytotoxic agents function by interfering with DNA replication or repair, or by causing DNA damage, either directly or indirectly. Two major forms of DNA damage caused by antimicrobial and cytotoxic agents are: (1) double-stranded DNA breaks and (2) stalled replication forks.

For example, fluoroquinolones (FQs), e.g., ciprofloxacin, function by interfering with the bacterial type II DNA topoisomerases: DNA gyrase encoded by gyrA and gyrB and topoisomerase IV (encoded by parC and pare (Drlica, K., and Zhao, X., Microbiol Mol Biol Rev. 61:377-392 (1997)). Both of these topoisomerases function by forming protein-bridged DNA double strand breaks (DSBs), manipulating DNA strand topology, and finally rejoining the ends of the DNA. Ciprofloxacin and other FQs reversibly bind to the protein-bridged DSB intermediates and inhibit the rejoining of the DNA ends. Cell death results from the creation of free DSBs when the topoisomerase dissociates from the DNA without rejoining the DNA ends (Drlica, K., and Zhao, X., Microbiol Mol Biol Rev. 61:377-392 (1997)) when DNA replication is inhibited by covalent DNA-protein complexes (Khodursky, A. B. and Cozzarelli, N. R., J. Biol Chem. 273:27668-27677 (1998)), and potentially by the induction of suicide proteins (reviewed by Drlica and Hooper, Mechanisms of Quinolone Action, in Quinolone Antimicrobial Agents, D. C. Hooper and E. Rubinstein, eds).

In addition, certain other antimicrobial and cytotoxic agents, such as trimethoprim, cause DNA damage by interfering with thymine biosynthesis, in a process referred to as “thymineless death,” which involves both single- and double-stranded DNA breaks. DNA damaged by thymine starvation is a substrate for DNA repair processes, including recombinational repair. Mutations in recBC(D) recombinational repair genes increase sensitivity to thymineless death (Ann. Rev. Microbiol. 52:591-625 (1998)).

Additionally, DSB's occur in bacterial genomes during in vivo infection due to the action of DNA damaging agents such as nitric oxide and oxygen radicals produced by the innate immune system (Ann. Rev. Imm. 14; 323-350, 1997). Recombinational repair has been shown to be critical for survival of E. Coli exposed to nitric oxide (J. Bact. 183:131-138, 2001), and the RecBCD pathway has been shown to be absolutely essential for virulence of Salmonella (J. Bact. 184:592-595, 2002). Thus, RecA or RecBC inhibitors should augment the activity of not only fluroquinolones but most or all antibiotics that are used to treat bacterial infections in which the pathogenic organism incurs DSB due to the action of nitric oxide or oxygen radicals produced by the innate immune system.

Without wishing to be bound to a particularly theory, the present invention establishes that microorganisms and cells utilize a variety of DNA repair pathways to repair different forms of DNA damage caused by anitmicrobial and cytotoxic agents, e.g., double-stranded DNA breaks and stalled replication forks, thereby permitting a microorganism or cell to survive in the presence of such antimicrobial and cytotoxic agents. Therefore, treating a cell with an inhibitor of a DNA repair or replication pathway enhances its sensitivity to an antimicrobial or cytotoxic agent, including those that cause DNA damage or interfere with DNA replication or repair.

Accordingly, the compounds and methods of the present invention may be used to target any DNA repair or replication pathway, in any microorganism or cell, including, e.g., mammalian cells. Microorganisms and cells possess a variety of different DNA repair mechanisms and pathways, including both homologous and non-homologous recombination-mediated pathways, in addition to non-recombination-based pathways. These DNA repair pathways are utilized during both normal cellular processes, such as DNA replication, as well as in response to DNA damaging agents, such as antimicrobial and cytotoxic compounds.

Repair of double-stranded DNA breaks is accomplished, in certain instances, via homologous recombination-mediated double-stranded break repair, including, e.g., RecBC(D)-mediated homologous recombination and RecFOR-mediated homologous recombination, non-homologous recombination-mediated double-stranded DNA break repair, and non-homologous end joining. In addition, repair or rescue of stalled replication forks is accomplished, in certain instances, via recombination-dependent replication fork repair and primosome reassembly. During DNA synthesis, replication fork progression on chromosomes can be impeded by DNA lesions, DNA secondary structures, or DNA-bound proteins. Elements interfering with the progression of replication forks have been reported to induce rearrangements and/or render homologous recombination essential for viability, in all organisms from bacteria to human.

DNA repair and replication pathways utilized by different microorganisms and cells, including mammalian cells, have been well-defined in the art, and a host of polypeptides involved in these processes and pathways have been identified. The invention contemplates compounds and related methods that reduce the activity and/or expression of any of such polypeptides, thereby inhibiting the activity of any such DNA repair or replication pathway, including but not limited to those specifically described herein.

Bacteria (and other microorganisms and cells) respond to low concentrations of DSBs using several DNA repair pathways. If homologous DNA is present, as is the case in a significant percentage of cells in a bacterial population, bacteria can repair DSBs by homologous recombination (HR) (J. Mol. Biol. 116; 81-98), including HR mediated by either RecBC(D) or RecFOR.

In E. coli, HR is mediated, in part, by RecBC(D). This heterotrimeric protein complex results from the association of RecB, RecC, and RecD. However, since RecD appears to be dispensable for at least some types of HR, including that induced by ciprofloxacin, the complex is referred to as RecBC(D). The RecBC(D) nuclease/helicase loads at the DSB and simultaneously degrades and unwinds the duplex while loading RecA onto the single-stranded DNA (ssDNA) of the nascent 3′-overhang. In this context, RecA forms filaments that promote strand invasion of the ssDNA into a homologous sequence, ultimately restoring an intact chromosome through a synthesis-dependent strand annealing, or DSB repair-like mechanism (Aguilera, A., Trends Genet. 17:318-21 (2001)).

In other instances, HR is mediated, in part, by RecFOR, a heterotrimeric protein complex composed of RecF, RecO, and RecR. In this pathway, RecF helps load RecA onto ssDNA, and RecO and RecR appear to play accessory roles. While this pathway appears less able to mediate HR in response to ciprofloxacin, and is generally associated with additional mutations in sbcA, sbcB, and sbcC, it is a pathway involved in processing the damage that underlies UV sensitivity and “thymineless death.”

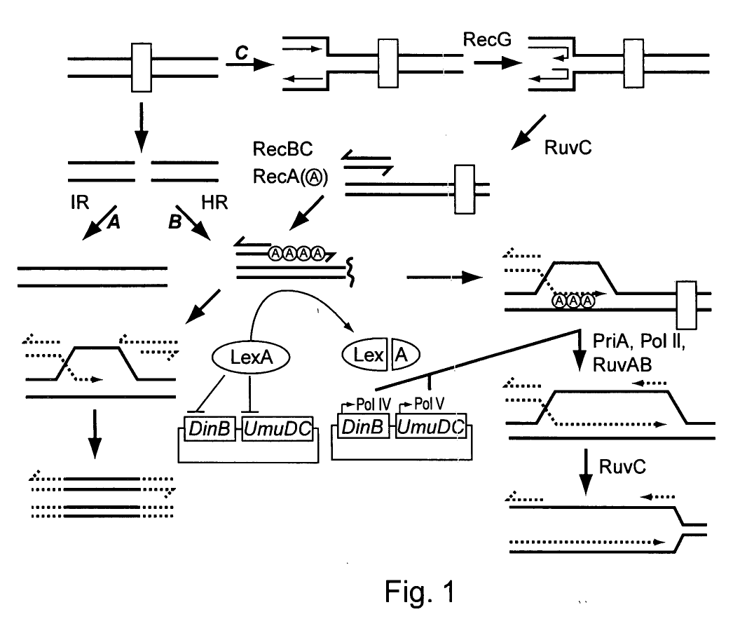

Additionally, bacteria (and other organisms and cells) respond to low concentrations of proteins bound to DNA, thereby inhibiting DNA replication, by recombination-dependent replication fork repair, which is a variant of HR (McGlynn, P., Lloyd, R. G., and Marians, K. J., Proc Natl Acad Sci USA., 98:8235-8240 (2001)). For example, one consequence of inhibition of type II topoisomerase by fluoroquinolones, such as ciprofloxacin, is the stalling of replication forks when they encounter the fluoroquinolone:topoisomerase:DNA complex. As illustrated in FIG. 1, these stalled forks are repaired by recombination-dependent replication fork repair. The stalled forks are regressed, possibly by RecG (Robu, M. E., Inman, R. B., and Cox, M. M., J Biol Chem., 279:10973-10981 (2004) and McGlynn, P., and Lloyd, R. G., Trends Genet., 18:413-419 (2002)) to form a Holliday junction-like structure that is recognized and cleaved by RuvC (Lovett, S. T., Hurley, R. L., Sutera, V. A. Jr, Aubuchon, R. H., Lebedeva, M. A., Genetics, 160:851-9 (2002)) to produce a double stranded end (DSE) and a nicked double stranded duplex. After the DSE is processed by RecBC(D), a RecA-ssDNA filament is formed that invades the homologous region of the nicked duplex. The resultant D-loop structure contains a primed template capable of initiating what will become leading strand synthesis of a new replication fork. An important step in the process of recombination-dependent replication fork repair is replication restart, or primosome reassembly, which is primed by the primosome complex. The primosome consists of DnaG primase, DnaB helicase, PriA, PriB, PriC, DnaC, and DnaT.

These repair strategies, many of which rely heavily on the function of the RecBC(D) helicase/nuclease complex, are thought to enable bacterial survival in the presence of low concentrations of antimicrobial and cytotoxic agents that cause DNA damage, such as ciprofloxacin. Resistance to higher concentrations requires multiple stepwise mutations in chromosomal genes (Drlica, K., and Zhao, X., Microbiol Mol Biol Rev. 61:377-392 (1997); Gibreel, A., et al., Antimicrob. Agents Chemother. 42:3276-3278 (1998); Kaatz, G. W., Seo, S. M., and Ruble, C. A., Antimicrob. Agents Chemother. 37:1086-1094 (1993); Yoshida, H., et al., J. Bacteriol. 172: 6942-6949 (1990); Poole, K., Antimicrob. Agents Chemother. 44:2233-2241 (2000); Kern, W. V., Oethinger, M., Jellen-Ritter, A. S., and Levy, S. B., Antimicrob Agents Chemother. 44:814-820 (2000); and Fukuda, H., Hori, S., and Hiramatsu, K., Antimicrob. Agents Chemother. 42:1917-1922 (1998)). Indeed, virtually all bacterial resistance to ciprofloxacin results from mutations in chromosomal genes (Everett, M. J., Jin, Y. F., Ricci, V., and Piddock, L. J. V., Antimicrob. Agents Chemother. 40:2380-2386 (1996); Deguchi, T., et al., Antimicrob. Agents Chemother. 41:1609-1611 (1997); Kanematsu, E., Deguchi, T., Yasuda, M., Kawamura, T., Nishino, Y., and Kawada, Y., Antimicrob. Agents Chemother. 42:433-435 (1998); and Wang, T., Tanaka, M., and Sato, K., Antimicrob. Agents Chemother. 42:236-240 (1998)). This is also the case for other synthetic and semi-synthetic antibiotics such as Rifampicin. Of the many resistance cases studied, clinical resistance to FQs by plasmid transfer has been reported only once (Martinez, J. L., Alonso, A., Gómez-Gómez, J. M., and Baquero, F., J. Antimicrob. Chemother. p. 42 (1998)). However, the plasmid alone imparted only a low level of resistance to ciprofloxacin, and chromosomal mutations were still required to attain high, clinically relevant resistance.

The location and nature of many of the ciprofloxacin (and other FQs) resistance-conferring mutations have been characterized and occur in the “quinolone resistance determining region” (QRDR). The primary mutations conferring resistance occur in two genes encoding the two molecular targets of ciprofloxacin, including, e.g., the gyrA gene encoding the alpha subunit of DNA gyrase (typically, the primary target in gram-negative bacteria such as E. coli, N. gonorrhoeae, K. pneumoniae, and C. trachomatis) or in the parC gene, encoding a subunit of topoisomerase IV (typically, the primary target in gram-positive bacteria including S. aureus, S. pneumoniae, and E. faecalis). The highest resistance, however, is conferred by mutations in both genes, combined with mutations in genes affecting outer membrane permeability or export through an active efflux system (Köhler, T., et al., Antimicrob. Agents Chemother. 41:2540-2543 (1997)).

Because the QRDR of gyrA and/or parC genes correspond to the DNA binding site of the topoisomerases, in addition to preventing ciprofloxacin binding, the mutations also interfere with DNA binding. Without wishing to be bound to a particular theory, it is understood according to the present invention that these mutations may also cause the topoisomerases to prematurely dissociate from the DNA before rejoining the cleaved DNA strands. Thus, these mutations may impose a liability on the cells harboring them by creating DSBs that must be repaired by HR, thereby making these cells dependant on RecBC(D) for viability. This is consistent with the data of Gari et al. (Gari, E., Bossi, L., and Figueroa-Bossi N., Genetics 159:1405-1414 (2001)), which demonstrates that a temperature sensitive allele of gyrA that mimics low level quinolone treatment at the nonpermissive temperature due to compromised gyrase activity are highly dependent on RecA and RecBC(D) for viability at the nonpermissive temperature.

Importantly, the specific residues of gyrA (i.e., S83 and D87) and parC (i.e., S80) that are frequently mutated in response to FQ selection are similar in both gram negative and gram positive bacteria. Thus, the observations described herein for E. coli may be generalized to other bacterial species.

Therefore, as described generally above, aspects of the present invention are based, in part, on the discovery that E. coli having mutations associated with antibiotic resistance (e.g., gyrA and parC mutations) utilize double-stranded DNA break repair and stalled replication fork repair pathways, including, e.g., RecBC(D)-mediated HR-based DNA repair for survival. Without being bound to any one molecular interpretation, it is understood that while these mutations confer antibiotic resistance, they also compromise their encoded enzyme's ability to carry out its normal functions. Accordingly, the present invention establishes that HR-based DNA repair and replication restart pathways are important for the survival of microorganisms and cells having compromised gyrase and topoisomerase IV activities, including those associated with drug resistance. Furthermore, certain aspects of the present invention are based upon the related discovery that RecBC(D)-mediated HR and replication restart (including primosome reassembly) are important for bacterial survival at even low levels of fluoroquinolones (i.e., at or below the MIC or MBC), and establish that inhibition of double-stranded DNA break repair, e.g., RecBC(D)-mediated homologous recombination, or stalled replication fork rescue or repair, e.g., recombination-dependent replication fork repair and primosome reassembly, causes bacteria to become hypersensitive to certain antimicrobial agents.

As described in detail in the Examples section, fundamental discoveries underlying the present invention were first made using the model organism E. coli, by probing the interdependence of gyrase, homologous recombination enzymes, and ciprofloxacin. For example, as detailed in Example 4, a strain containing a temperature sensitive mutant of RecBC(D) was used to demonstrate that drug-resistant cells are more sensitive to drug when RecBC(D) activity is impaired. In addition, E. coli strains containing mutations in recA, recB, recG or priA, ruvA, ruvB or ruvC were also increasingly sensitive to fluoroquinolones (Examples 1 and 2).

Of course, it is understood that these findings in E. coli are not limited to this bacterial species. A variety of other bacterial species contain recBC(D) homologues, including, e.g., P. aeuruginosa, Salmonella, S. pneumoniae, S. aureus, methicillin resistant S. aureus, Acinetobacter or B. anthracis. Accordingly, treatment with an inhibitor of a DNA repair or replication pathway, e.g., RecBC(D)-mediated homologous recombination or replication restart, in combination with ciprofloxacin or other FQs, or other DNA damaging agents, would also kill these strains, whether they had evolved to be, or were engineered to be, resistant to such DNA damaging agents. In addition, inhibitors of DNA repair or replication should also sensitize these species to antimicrobial agents.

In addition, while these discoveries were first made in bacteria, it is clearly applicable to other cells and organisms, including mammals. Basic mechanisms and certain components of DNA repair and replication pathways are generally conserved from bacteria to eukaryotic cells. In addition, mechanisms of drug action and the acquisition and maintenance of drug resistance, are also shared from bacteria to eukaryotic cells. Thus, the mechanisms of combating drug resistant microorganisms and enhancing drug sensitivity described herein, exemplified in the context of bacteria, are applicable to a wide range of microorganisms and eukaryotic cells, including mammalian cells.

However, in another aspect, differences in activities and physical properties between eukaryotes and bacteria or other pathogens can be used to develop inhibitors that are specific or preferential for bacteria or other infective agents. This is beneficial in the context of inhibitors that are administered to treat, e.g., bacterially, virally or other pathogen-caused diseases in patients, e.g., in human or other mammalian patients. Inhibitors that are differentially active in pathogen repair and replication pathways as compared to patient cells can cause fewer side effects (e.g., unwanted patient cell cytotoxicity) than inhibitors that are equally active against patient repair and replication pathways. Thus, generally, inhibitors can be screened for differential activity against a target pathogen organism as compared to activity against patient cells. Inhibitors that display differential activity, e.g., greater activity against the target pathogen, as compared to patient cells, are preferred inhibitors.

The fundamental role of DNA repair and replication pathways in maintaining cell viability in the presence of mutations associated with drug resistance, or in the presence of drugs that interfere with DNA replication or repair, or cause DNA damage, as discovered according to the present invention, combined with the knowledge of these pathways and their components in many cells types, e.g., bacteria, fungi, and mammalian cells, provides a sound scientific basis for applying the compounds and methods of the present invention to treat a wide variety of drug-resistant microorganisms and cells, and also enhance drug sensitivity of various microorganisms and cells, including mammalian cells.

Indeed, impaired activity of topoisomerases has been shown to result in an increased reliance on HR in eukaryotes, in addition to prokaryotes. HR has been extensively studied in the model organism S. cerivisae. In the case of a DSB, the MRX complex (comprised of Mre11, Rad50 and Xrs2) first binds and then recruits the Tell checkpoint kinase via an interaction between Xrs2 and Tel1. The MRX complex is required to process ‘dirty’ DSEs, such as those that arise in response to ionizing radiation, but not those resulting from endonuclease activity. The MRX complex then dissociates and 5′-3′ resection is initiated by an unknown nuclease(s), producing a 3′-overhang that is coated with replication protein A (RPA), which acts to preserve the integrity of the 3′-overhang until it is displaced during S-phase by Rad52. Rad52 plays a central role in single-strand annealing (SSA), gene conversion (GC), and break induced recombination (BIR). If the exposed ssDNA overhangs contain sufficient homology, Rad52, possibly along with its homolog Rad59, facilitates repair by SSA. For GC and BIR, Rad52 recruits Rad51, the homolog of the bacterial recombination mediator RecA, to DSEs where it catalyzes strand invasion of a homologous duplex with concomitant displacement of the strand of the same polarity (forming an intermediate referred to as a displacement structure or D-loop). The invading strand primes DNA synthesis using the homologous sequence, ultimately creating an intact sequence at the site of a break or restoring a processive replication fork. While the activities of Rad54, Rad55, and Rad57 are sometimes not required, they appear to mediate the most efficient forms of HR, possibly by helping to stabilize Rad52-ssDNA nucleoprotein filaments. The helicases Srs2 and Sgs1 are understood to help form suitable recombination intermediates and/or to help resolve these intermediates after recombination-dependant DNA replication.

In mammalian cells, HR is an important mechanism for repairing blocked or stalled replication forks and is thought to play an important role in the repair of double-stranded DNA breaks. Consequently, inhibitors of proteins such as Rad52, Rad55, BRACA1, and BRACA2 are predicted to synergize with DNA damaging agents, topoisomerse poisons and other agents that lead to blockage of replication forks. Inhibition of the production of these proteins by, e.g., RNAi-based mechanisms, should have similar effects.

Of course, not all DNA repair pathways are mediated by HR. Additional aspects of the present invention are based on the understanding that nonhomologous end joining (NHEJ) or nonhomologous recombination (NHR) are major mechanisms for repair of DSB in mammals. NHEJ generally repairs DSB by performing a microhomology search for regions with microhomology (about 2 to 20 bases) to a DSB and by repairing the lesion in a NHR reaction. Major components of this pathway are DNA protein kinase (DNA-PK), the Ku70 and Ku86 proteins, and the XRCC4 protein. The Ku proteins form a heterodimeric helicase that binds with high affinity to double stranded ends of DNA and recruits DNA-PK. Subsequently, Ku unwinds the DNA and promotes repair either by homology dependent or homology independent pathways (Rathmell and Chu, DNA Double-Strand Break Repair, Chapter 16 or Nickoloff, J. A. and Hoekstra, M. F. in DNA Damage and Repair, Humana Press, Totowa, N.J., 1998). Cells deficient in XRCC4, Ku86 or DNA-PK are hypersensitive to ionizing radiation.

As this is a major pathway for DSB repair in mammals, inhibitors of proteins this pathway are predicted to hypersensitize cells to DNA damaging agents that cause DSB. Without being limited to any particular theory, inhibitors of the Ku proteins (Ku70 and Ku86), DNA-PK or XRCC4 are understood to sensitize mammalian cells to DNA damaging agents and, thus, may be used in combination with treatment regimes, such as treatment with chemotherapeutics or ionizing radiation, that generate DNA damage, to enhance treatment sensitivity or kill resistant cells.

In addition, the observation that topoisomerases that have accumulated mutations in response to topoisomerase poisons have increased reliance on mechanisms that repair DSB and stalled replication forks, due to an elevated level of DSB and stalled replication forks in cells harboring such mutant topoisomerases, may be generalized to eukaryotes. In this regard, such effects are expected to be dominant and, thus, eukaryotic cells bearing somatically selected mutations in their topoisomerases are sensitive to drugs that inhibit the relevant repair pathways.

In light of the fundamental discoveries related to the role of DNA repair and replication pathways in drug sensitivity and resistance, the present invention provides both compositions and methods for inhibiting DNA repair and replication pathways, including, but not limited to those involving double-stranded DNA break repair and stalled replication fork rescue or repair, and novel uses for such inhibitors. The compositions of the present invention include a broad spectrum of inhibitors of DNA repair or replication, including inhibitors of any of the DNA replication or repair pathways or mechanisms referred to herein, any and all of which may be used to inhibit one or more activities of a polypeptide associated with DNA repair or replication. Furthermore, these compositions may be used to combat drug-resistant microorganism and cells, as well as to enhance the sensitivity of both sensitive and resistant cells to antimicrobial and cytotoxic agents, including, e.g., antibiotics and chemotherapeutics.

The compositions and methods of the present invention are applicable to a broad range of drug-resistant microorganisms and cells, including, e.g., bacteria, viruses, fungi, protozoa and eukaryotic cells of higher organisms, such as mammals. In addition, the invention is applicable to a wide variety of antimicrobial and cytotoxic agents or compounds, including, but not limited to, those that target cellular components of a DNA replication or repair pathway or cause DNA damage, either directly or indirectly.

The following definitions are provided to more clearly define terms used in describing the present invention. As used herein, the following phrases are defined as follows:

“DNA repair or replication” refers to any and all biological processes and pathways involved in either DNA repair or replication in any organism or cell, including, e.g., microorganisms and mammalian cells. Such processes and pathways include, but are not limited to, homologous recombination-mediated DNA repair, recombination-dependent DNA replication, repair of DNA double-stranded breaks, repair or rescue of stalled replication forks, replication restart, primosome reassembly, RecBC(D)-mediated homologous recombination, RecFOR-mediated homologous recombination, nonhomologous recombination, nonhomologous end joining, single-stranded annealing, gene conversion, and break-induced recombination, as these terms are generally understood in the art and described herein.

“Double-stranded DNA break repair” includes any and all biological pathways or processes involved in the repair of double-stranded DNA breaks, including, but not limited to, homologous recombination-mediated pathways, e.g., RecBC(D)-mediated HR and RecFOR-mediated HR, non-homologous recombination pathways, e.g., illegitimate recombination (IR), and non-homologous end joining (NHEJ).

“Stalled replication fork rescue or repair” refers to any and all biological pathways or processes involved in the repair of stalled, blocked, or collapsed replication forks, including but not limited to replication restart, recombination-dependent replication fork repair, and primosome reassembly.

“Homologous recombination” is reciprocal or non-reciprocal recombination between DNA sequences that have a high degree of sequence similarity. Homologous recombination is important for a variety of functions in all cell types. Homologous recombination is important for, but not necessarily limited to, the repair of DNA damage, DNA double stranded breaks, and DNA polymerase replication forks that have stalled or collapsed. In bacteria, homologous recombination is also part of conjugation and is required for bacteriophage replication. In higher organisms, it is important for meiotic crossovers, which are responsible for the rearrangement of alleles, as well as being necessary for proper chromosome segregation. It is important for mating type switching in yeast, generation of diversity in some mammalian antibody gene repertoires, and epitope class switching in many organisms such as malaria.

“Homologous recombination-mediated DNA repair” refers to any DNA repair pathway involving homologous recombination, including, e.g., recombination-dependent DNA replication, RecBC(D)-mediated homologous recombination, and RecFOR-mediated homologous recombination.

“Recombination-dependent DNA replication” refers to DNA repair pathways that involve both DNA recombination and replication. Typically, this involves DNA replication where the polymerase substrate is a recombination intermediate, such as Holliday junction or a displacement loop (D-loop). A D-loop is a recombination intermediate resulting from strand invasion of a DNA strand, which will serve as a primer for DNA replication, into a region of duplex DNA that has sufficient sequence homology. For DSB repair, the newly generated 3′-ends of the DNA may, thus, recover the sequence information in the region of the DSB. This process is typically mediated in E. coli by (but not necessarily only by) RecBC(D) and the recombination mediator RecA (or their homologous proteins in bacteria other than E. coli) and in eukaryotes by Mre11, Rad50, Xrs2 and the recombination mediator proteins Rad51, Rad52, Rad54, Rad55, Rad57, Rad59, Srs2, and Sgs1.

“Recombination-dependent replication fork repair” refers to mechanisms for repairing or restarting stalled, blocked, or collapsed replication forks involving homologous recombination.

“Replication restart” is an important step in the process of recombination-dependent replication fork repair is replication restart, or primosome reassembly, which is primed by the primosome complex. The primosome consists of DNAG primase, DNAB helicase, PriA, PriB, PriC, DnaC, and DnaT.

“Non-homologous end joining” is a process wherein DSB are repaired by joining them to another DSB with requirements for microhomology (about 2 to about 20 base pairs). Polypeptides involved in mammalian non-homologous end joining include, e.g., DAN-PK, Ku70, Ku86, and XRCC4.

“Non-homologous recombination” refers to a non-reciprocal recombination event, e.g., when NHEJ joins two DSB.

“Microrogansim,” as used herein, refers to any organism of microscopic or submicroscopic size, including, e.g., a bacterium, a fungus, a virus, and a protozoan, as well as other small organisms, such as certin fungi and nematodes.

A. Inhibitors

The present invention establishes that DNA repair and replication pathways, including those described above, are involved in cellular responses to antimicrobial and cytotoxic agents or compounds, including, but not limited to, antibiotics and chemotherapeutic drugs. Furthermore, the present invention demonstrates that DNA repair and replication pathways play a fundamental role in both drug resistance and drug sensitivity, and that inhibition of DNA repair or replication pathways can both enhance drug sensitivity and kill drug resistant microorganisms and cells. In addition, DNA repair and replication pathways are involved in mutagenesis associated with drug resistance. Therefore, the present invention establishes that inhibiting a DNA repair or replication pathway results in enhanced drug sensitivity, killing of drug resistant cells, and inhibition of mutagenesis and associated development of drug resistance.

More generally, modulators of DNA repair and replication pathways (whether inhibitors or activators) can enhance or suppress drug sensitivity, enhance killing or protection of drug resistant cells, and/or inhibit or enhance mutagenesis and associated development of drug resistance. For convenience, the following discussion focuses on inhibitors, but it will be appreciated that similar discussion applies to modulators generally.

Accordingly, the compositions and methods of the present invention are directed to any and all inhibitors of a DNA repair or replication pathway, or polypeptide associated with such a pathway. In particular aspects of the present invention, therefore, an inhibitor targets a pathway associated with the repair of double-stranded DNA breaks, or an inhibitor targets a pathway associated with the repair of stalled replication forks.

In particular embodiments, an inhibitor of the present invention targets a repair or replication pathway associated with double-stranded DNA break repair. In certain, more specific embodiments related to double-stranded DNA break repair, an inhibitor targets homologous recombination, non-homologous recombination or non-homologous end joining. Specific embodiments of homologous recombination include, but are not limited to, RecBC(D)-mediated homologous recombination and RecFOR-mediated homologous recombination.

In other embodiments, an inhibitor of the present invention targets a repair or replication pathway associated with stalled replication fork rescue or repair. Specific embodiments of stalled replication fork rescue or repair include, but are not limited to, recombination-dependent replication fork repair and replication restart (or primosome reassembly).

In certain embodiments, an inhibitor targets a polypeptide associated with a DNA replication or repair pathway involving homologous recombination. However, in other embodiments, an inhibitor targets a polypeptide associated with a DNA repair or replication pathway that does not involve homologous recombination. Inhibitors of the present invention that reduce the activity of one or more polypeptides associated with either homologous recombination or non-homologous recombination may be referred to as “recombinicides.” In particular embodiments, the present invention is directed to inhibitors of RecBC(D)-mediated homologous recombination, RecFOR-mediated recombination, homologous recombination-mediated DNA repair, recombination-dependent replication fork repair, replication restart or primosome reassembly, gene conversion, single-strand annealing, break-induced recombination, and/or non-homologous end joining, and polypeptides associated with any of these pathways. As used herein, the term DNA repair or replication encompasses, but is not limited to, any and all of these biological pathways.

In general, inhibitors act by reducing the activity or expression of one or more polypeptides associated with a DNA repair or replication pathway. In various embodiments, an inhibitor reduces one or more activities of a polypeptide by at least 20%, 30%, 40%, 50%, 60%, 70%, 80%, 90%, or 100%, as compared to the activity in the absence of the inhibitor. In particular embodiments, an inhibitor reduces expression of a polypeptide by at least 20%, 30%, 40%, 50%, 60%, 70%, 80%, 90%, or 100%. In certain embodiments, an inhibitor specifically binds to a target polypeptide or a polynucleotide encoding a target polypeptide.

Inhibitors can act directly, e.g., by reducing the activity or expression of a polypeptide required for DNA repair or replication, or indirectly, e.g., by increasing the activity or expression of a polypeptide that blocks DNA repair or replication.

In certain embodiments, the invention is directed to inhibitors of RecBC(D)-mediated homologous recombination or similar biological pathways in other organisms and cells. Thus, in specific embodiments, an inhibitor reduces one or more biochemical, enzymatic or biological activities of a polypeptide associated with RecBC(D)-mediated homologous recombination, such as, e.g., RecB, RecA, PriA, RuvA, RuvB, RuvC, RecG, RecC, RecD, RecF, UvrD, or Rep helicase, or a variant, homolog, or ortholog thereof.

In one embodiment, an inhibitor reduces one or more activities of RecBC(D). RecBC(D) is a multi-functional enzyme complex that processes DNA ends resulting from a double-strand break. RecBC(D) is a heterotrimeric complex of three polypeptide subunits, RecB, RecC and RecD. The complex contains both nuclease and helicase activities involved in homologous recombination. RecBC(D) possesses three different activities, including nuclease, helicase, and ATPase activities. RecBC(D) acts as a bipolar helicase that separates the duplex into its component strands and concurrently digests them until encountering a recombinational hotspot (Chi site). The nuclease activity is then attenuated, and RecA is loaded onto the 3′ tail of the DNA. Studies to determine the contribution of each of the subunits to the enzymatic activity of RecBC(D) demonstrated that RecD is necessary for nuclease activity. The active site for nuclease activity is known to be in the RecB domain, although it is inactive when separated from RecD. RecBC possesses helicase and concurrent ATPase activities. Accordingly, in various embodiments, an inhibitor reduces one or more nuclease, helicase, or ATPase activity of RecBC(D).

In related embodiments, an inhibitor reduces one or more activities of any of RecB, RecC, or RecD. Point mutations have been identified that selectively knockout either the helicase or nuclease activities of RecB or RecD, respectively. The active site for nuclease activity has been shown to lie within the RecB domain, although it is thought to be inactive when separated from RecD. Pyridoxal phosphate has been shown to target the DNA binding site of RecBC(D), while the γ-protein encoded by bacteriophage lambda was shown to inhibit the nuclease activity of RecBC, apparently through an interaction with RecD. Helicase activity of RecBC(D) is impaired by mutation of RecB (RecB K29Q) or RecD (RecDK177Q) (Dillingham, M. S. et al., Nature 893-897 (2003) and Taylor A. F. and Smith G. R., Nature 889-893 (2003)). Thus, in particular embodiments, an inhibitor binds or interferes with binding to a region of RecB comprising amino acid residue K29 or a region of RecD comprising amino acid residue K177. In a particular embodiment, an inhibitor reduces RecB helicase activity.

The crystal structure of RecBC(D) bound to DNA has recently been determined, and functional regions and sites within RecBC(D) have been identified (Singleton, M. R. et al., Nature 432:157-8 (2004)). RecBC(D) is a bipolar helicase that splits the DNA duplex into its component strands and digests them until encountering a recombinational hotspot (Chi site). The nuclease activity is then attenuated, and RecBC(D) loads RecA onto the 3′ tail of the DNA. When RecBC(D) is bound to a DNA substrate, the DNA duplex is split across the RecC subunit to create a fork with the separated strands each heading towards different helicase motor subunits. The strands pass along tunnels within the complex, both emerging adjacent to the nuclease domain of RecB. Passage of the 3′ tail through one of these tunnels provides a mechanism for the recognition of a Chi sequence by RecC within the context of double-stranded DNA. Gating of this tunnel suggests how nuclease activity might be regulated. In certain embodiments, an inhibitor binds at or near a functional region of RecBC(D), thereby inhibiting a functional activity. Examples of functional regions include, e.g., the chi cutting site, the tunnels within the complex, and the nuclease domain of recB. For example, in some embodiments the inhibitor is structurally configured to bind to a target functional region. Given that the crystal structure of the functional regions are available, the structural configuration of the cognate binding region of the inhibitor can, in some embodiments, be inferred. In particular embodiments, an inhibitor reduces activity of either the endonuclease or exonuclease activity of RecB.

In another embodiment, an inhibitor reduces one or more activities of RecA. The RecA protein is a critical enzyme in the process of homologous recombination, as it catalyzed the pairing of ssDNA with complementary regions of dsDNA. The RecA monomers first polymerize to form a helical filament around ssDNA. During this process, RecA extends the ssDNA by 1.6 angstroms per axial base pair. Duplex DNA is then bound to the polymer. Bound dsDNA is partially unwound to facilitate base pairing between ssDNA and duplexed DNA. Once ssDNA has hybridized to a region of dsDNA, the duplexed DNA is further unwound to allow for branch migration. RecA has a binding site for ATP, the hydrolysis of which is required for release of the DNA strands from RecA filaments. ATP binding is also required for RecA-driven branch migration, but non-hydrolyzable analogs of ATP can be substituted for ATP in this process, suggesting that nucleotide binding alone can provide conformational changes in RecA filaments that promote branch migration. Therefore, in one embodiment, an inhibitor binds to or inhibits binding of ATP to a RecA ATP binding site or inhibits RecA binding to single-stranded DNA. Thus, an inhibitor may reduce the ATPase or DNA binding activity of RecA.

In other embodiments, an inhibitor reduces one or more activities of RuvC, RuvAB or a subunit thereof. These proteins encode both helicase and branch migration capabilities. Introduction of mutant RuvB domains in the presence of wild type RuvB impedes branch migration but not ATP hydrolysis.

In another embodiment, an inhibitor reduces one or more activities of RecG. RecG is a helicase that promotes branch migration of Holliday junctions. It is believed that the active form is a monomer. A mutant RecG with the substitution K302 to either A or R was shown to lack helicase activity. RecG appears to work in parallel with RuvAB. Deletion of recG hypersensitizes E. coli to ciprofloxacin (Example 1).

In a further embodiment, an inhibitor reduces one or more activities of RecF. RecF binds both DNA and ATP, although no clear enzymatic activity has been defined. Without wishing to be bound to any particular theory, it is believed that RecF may serve to maintain arrested replication forks and assist in loading of RecA, since overexpression of RecA compensated for RecF deficiency.

In another embodiment, an inhibitor reduces one or more activities of UvrD helicase. UvrD dismantles the RecA nucleoprotein filament. UvrD has a role in UV repair where it allows the removal of a 12-nt-long DNA segment containing a UV lesion, after its incision by the combined action of UvrA, UvrB and UvrC (Orren D K, et al., J Biol Chem 267: 780-788 (1992)). UvrD is involved in mismatch repair, where it promotes the removal of the DNA segment containing the erroneous nucleotide after its incision by the combined action of MutS, MutL and MutH Modrich P., Science 266: 1959-1960 (1994). UvrD is related to the yeast Srs2 helicase.

In additional embodiments, an inhibitor reduces the activity or expression of one or more polypeptides associated with recombination dependent fork repair or replication restart, such as a component of the primosome. Thus, in particular embodiments, an inhibitor reduces the activity or expression of PriA, PriB, PriC, DnaC, or DnaT. Inhibitors of the primosome hypersensitive cells to fluoroquinolones based on preventing repair of stalled replication forks. Inhibitors that prevent the formation of a active primosome or inhibit the activity of the primosome also hypersensitive cells to other agents, such as rifampin and its analogs that give rise to blocked replication forks (stalled transcription complexes in the case of rifampin), since they prevent or reduce repair of stalled forks.

In one embodiment, an inhibitor reduces one or more activities of PriA. PriA is a key component of the system for priming DNA synthesis in E. coli. Null mutants are defective in homologous recombination and hypersensitive to chemical and physical mutagens such as radiation or free radicals. PriA is known to possess ATPase, helicase and primase activities. These two functions can be separated by a single point mutation K230R, which inactivates the helicase while retaining the primase activity. In various embodiments, an inhibitor reduces the activity of PriA helicase, primase, or ATPase activities.

As described above, RecFOR-mediated HR is an important repair pathway of the damage underlying UV sensitivity and “thymineless death.” Accordingly, inhibitors of this pathway can be used, alone or in combination with DNA damaging agents, e.g., trimethorprim or aminopterin, that impact these pathways, as well as members of the FQ class of drugs that cause damage that is processed by the RecFOR pathway, to reduce viability of drug-resistant microorganisms and cells and enhance sensitivity of both drug-resistant and drug-sensitive cells. Thus, in additional embodiments, an inhibitor of the present invention reduces the activity or expression of one or more polypeptides of the RecFOR pathway, such as, e.g., RecF, RecA, RecO, and RecR. In particular embodiments, the inhibitor reduces activity of the RecFOR pathway in the presence of a mutation in sbcA, sbcB, or sbcC.

In addition, DNA damaged by thymine starvation is a substrate for recombinational repair. Mutations in recBC(D) recombinational repair genes increase sensitivity to thymineless death (Ann. Rev. Microbiol. 52:591-625 (1998)). Thus, inhibitors of recombination enzymes, such as RecBC(D) and RecA are understood, according to the present invention, to hypersensitize bacteria and other microorganisms to thymine starvation or to blockers of thymine metabolism, such as trimethoprim.

The E. coli mazEF suicide cassette is reported to modulate thymineless death (J. Bact. 185:1803-1807 (2003)). This suicide cassette consists of a toxin (MazF and an antitoxin (MazE). Various stresses such as thymine starvation, blockage of thymine metabolism by trimethoprim or sulfonamides, or treatment with the antibiotics rifampin, chloramphenicol or spectinomycin, trigger this suicide module. It is believed that this suicide module also contributes to the mechanism of killing by other antibiotics, such as quinolones. Therefore, inhibitors, e.g., small molecules, that tip the balance of this trigger toward an excess of MazF, e.g., by inhibiting MazE expression or activity or enhancing MazF activity or expression hypersensitize bacteria to killing by these antibiotics. Accordingly, in particular embodiments, an inhibitor of the present invention reduces the activity or expression of MazE directly or indirectly by enhancing the activity of MazF.

In related embodiments, inhibitors reduce the expression or activity of a polypeptide associate with DNA repair or replication in a different microorganism or eukaryotic cell. For example, in particular embodiments, an inhibitor targets a polypeptide that is a homolog or functionally analogous polypeptide to any of those specifically identified herein, such as the AddAB complex in gram-positive bacteria (e.g., B. anthracis).

Inhibitors, in other embodiments, are targeted to one or more components of a mammalian DNA repair or replication pathway. Such pathways may be HR and non-HR pathways, such as, e.g., NHEJ or NHR. Accordingly, in particular embodiments, an inhibitor reduces the activity or expression of a polypeptide associated with a mammalian DNA repair pathway, such as, e.g., DNA-PK, Ku70, Ku86, or XRCC4.

In certain embodiments, an inhibitor reduces activity or expression of a polypeptide associated with a mammalian DNA repair or replication pathway, such as, e.g., a component of the MRX complex (i.e., Mre11, Rad50, and Xrs2), Tel1, replication protein A, Rad59, Rad51, Rad54, Rad55, Rad57, Srs2, or Sgs1. Inhibitors may inhibit the activity or expression of one or more other mammalian polypeptides, such as, e.g., BRCA1 or BRCA2.

Inhibitors may be characterized based upon the type of enzymatic, biochemical, or biological activity that they inhibit. Accordingly, in various embodiments, inhibitors reduce or inhibit an endonuclease, exonuclease, ATP-ase, helicase, DNA binding, or polymerase activity.

In general, inhibitors may be naturally-occurring or non-naturally occurring. In addition, an inhibitor may be isolated or purified and may also exist in a precursor form, e.g., in a form that is metabolized to produce an active inhibitor. As would be readily understood by one of skill in the art, an inhibitor may be any of a wide variety of different types of molecules, each type having been shown to be capable of possessing polypeptide inhibitory properties in various contexts. For example, in various embodiments, inhibitors comprise a nucleic acid, a polypeptide, a peptide, a peptidomimetic, a peptide nucleic acid (“PNA”), an antibody, a phage, a phagemid, or a small or large organic or inorganic molecule. Inhibitors further include salts, prodrugs, derivatives, homologs, analogs and fragments of any of these classes of molecules.

As illustrated in the following description of specific inhibitors, a wide variety of different types of molecules can be used as inhibitors. The skilled artisan would readily appreciate that polypeptide components associated with DNA repair and replication may be inhibited by many different mechanisms. For example, it is generally accepted that antibodies, or fragments thereof, can be generated that bind to a functional region of a polypeptide and inhibit its function. Similarly, it is understood that antisense and RNAi reagents can be produced that effectively prevent expression of a target polypeptide. Accordingly, the skilled artisan would appreciate that inhibitors of the present invention may be broadly defined based upon their inhibitory function, rather than their particular structural characteristics. Indeed, previously identified inhibitors of RecB include the molecule pyridoxal phosphate, as well as the lambda gam polypeptide (i.e., lambda gamma protein and its homologues, phage T7 gene 5.9 and its homologues, P22 phage encoded Abc1 and Abc2 and their homologues for example), thereby demonstrating that very different types of molecules can serve as effective inhibitors of RecB function.

In certain embodiments, an inhibitor reduces the expression or activity of a polypeptide associated with DNA repair, recombination, or replication in one type of microorganism or cell, but not in another. For example, an inhibitor may inhibit a DNA repair or replication pathway in a microorganism but not affect mammalian cells. Such inhibitors may be preferential in treating microbial infections in mammals.

1. Polynucleotide Inhibitors

In certain embodiments, inhibitors are polynucleotides capable of inhibiting one or more pathways and/or polypeptides associated with DNA repair or replication. The polynucleotide compositions of this invention can include genomic sequences, coding sequences, complementary sequences, extra-genomic and plasmid-encoded sequences, linear or circular polynucleotides, and vectors and smaller engineered gene segments that express, or may be adapted to express, proteins, polypeptides, peptides and the like. Such polynucleotides may be naturally isolated, or modified synthetically. Polynucleotides of the invention may be single-stranded (coding or antisense strand) or double-stranded, and may be DNA (genomic, cDNA or synthetic) or RNA molecules. In various embodiment, polynucleotide inhibitors are antisense RNA, ribozymes, or RNA interference reagents designed to specifically inhibit expression of a polypeptide involved in double-stranded DNA break repair or stalled replication fork rescue or repair, such as, e.g., RecB, RecA, PriA, RuvA, RuvB, RecG, RecA, RecC, and RecF. In addition, in particular embodiments, any of the various polynucleotide inhibitors described herein comprise a polynucleotide sequence corresponding to or complementary to a region of a gene encoding a component of a double-stranded DNA break repair or stalled replication fork rescue or repair pathway, including, e.g., RecA, RecB, RecC, RecG, PriA, RuvA, RuvB, or RuvF.

a. Antisense

In one embodiment, an inhibitor is an antisense RNA directed to a component of a DNA repair or replication pathway. Antisense oligonucleotides have been demonstrated to be effective and targeted inhibitors of protein synthesis, and, consequently, can be used to specifically inhibit protein synthesis by a targeted gene. Examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science 240:1544-6 (1988); Vasanthakumar and Ahmed, Cancer Commun. 1:225-32 (1989); Peris et al., Brain Res Mol Brain Res. 57:310-20 (1998); U.S. Pat. No. 5,801,154; U.S. Pat. No. 5,789,573; U.S. Pat. No. 5,718,709 and U.S. Pat. No. 5,610,288).

Therefore, in certain embodiments, the present invention relates to methods of providing oligonucleotide sequences that comprise all, or a portion of, any sequence that is capable of specifically binding to a polynucleotide sequence encoding a polypeptide involved in DNA repair or replication, or a complement thereof. In one embodiment, the antisense oligonucleotides comprise DNA or derivatives thereof. In another embodiment, the oligonucleotides comprise RNA or derivatives thereof. The antisense oligonucleotides may be modified DNAs comprising a phosphorothioated modified backbone. Also, the oligonucleotide sequences may comprise peptide nucleic acids or derivatives thereof. In each case, preferred compositions comprise a sequence region that is complementary, and more preferably, completely complementary to one or more portions of a target gene or polynucleotide sequence.

Methods of producing antisense molecules are known in the art and can be readily adapted to produce an antisense molecule that targets a gene encoding a component of a DNA repair or replication pathway. For example, antisense molecules may be chemically synthesized or expressed from an appropriate vector. Selection of antisense compositions specific for a given sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul. 15; 25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

b. Ribozymes

According to another embodiment of methods of the invention, ribozyme molecules are used to inhibit expression of a target gene or polynucleotide sequence encoding a polypeptide involved in DNA repair or replication. Ribozymes are RNA-protein complexes that cleave nucleic acids in a site-specific fashion. Ribozymes have specific catalytic domains that possess endonuclease activity (Kim and Cech, Proc Natl Acad Sci USA 84:8788-92 (1987); Forster and Symons, Cell 49:211-20 (1987)). At least six basic varieties of naturally occurring enzymatic RNAs have been described. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target-binding portion of an enzymatic nucleic acid, which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme may be advantageous over many technologies, such as antisense technology (where a nucleic acid molecule simply binds to a nucleic acid target to block its translation), since the concentration of ribozyme necessary to affect inhibition of expression is typically lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci USA 89:7305-9 (1992)). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. 20:4559-65 (1992). Examples of hairpin motifs are described by Hampel et al. (Eur. Pat. Appl. Publ. No. EP 0360257), Hampel and Tritz, Biochemistry 28:4929-33 (1989); Hampel et al., Nucleic Acids Res. 25:299-304 (1990) and U.S. Pat. No. 5,631,359. An example of the hepatitis δ virus motif is described by Perrotta and Been, Biochemistry 31:11843-52 (1992); an example of the RNaseP motif is described by Guerrier-Takada et al., Cell 35:849-57 (1983); Neurospora VS RNA ribozyme motif is described by Collins (Saville and Collins, Cell 6:685-96 (1990); Saville and Collins, Proc Natl Acad Sci USA 88:8826-30 (1991); Collins and Olive, Biochemistry 32:2795-9 (1993)); and an example of the Group I intron is described in (U.S. Pat. No. 4,987,071). Important characteristics of enzymatic nucleic acid molecules used according to the invention are that they have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Ribozyme activity can be optimized by altering the length of the ribozyme binding arms or chemically synthesizing ribozymes with modifications that prevent their degradation by serum ribonucleases (see e.g., PCT Publ. No. WO 92/07065: PCT Publ. No. WO 93/15187; PCT Publ. No. WO 91/03162; Eur. Pat. Appl. Publ. No. 92110298.4; U.S. Pat. No. 5,334,711; and PCT Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

c. RNAi Molecules

RNA interference methods using RNAi molecules also may be used to disrupt the expression of a gene or polynucleotide of interest, including a gene associated with DNA repair or replication. While the first described, RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., Molecular Biotechnology 24:111-119 (2003)). Accordingly, the invention includes the use of RNAi reagents comprising any of these different types of double-stranded molecules. In addition, it is understood that RNAi reagents may be used and introduced to cells in a variety of forms. Accordingly, as used herein, RNAi reagents encompasses any and all reagents capable of inducing an RNAi response in cells, including, but not limited to, double-stranded polynucleotides comprising two separate strands, i.e., a sense strand and an antisense strand, polynucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

In one particular embodiment, a dsRNA molecule that targets and induces degradation of a polynucleotide encoding a polypeptide involved in DNA repair or replication is introduced to a microorganism or cell. While the exact mechanism is not essential to the invention, it is believed the association of the dsRNA to the target gene is defined by the homology between the dsRNA and the actual and/or predicted mRNA transcript. It is also believed that this association will affect the ability of the dsRNA to disrupt the target gene. DsRNA methods and reagents are described, e.g., in PCT applications WO 99/32619, WO 01/68836, WO 01/29058, WO 02/44321, WO 01/92513, WO 01/96584, and WO 01/75164.

Double-stranded RNA-mediated suppression of gene and nucleic acid expression may be accomplished according to the invention by introducing dsRNA, siRNA or shRNA into cells or organisms. dsRNAs less than 30 nucleotides in length do not appear to induce nonspecific gene suppression, as described supra for long dsRNA molecules. Indeed, the direct introduction of siRNAs to a cell can trigger RNAi in mammalian cells (Elshabir, S. M., et al. Nature 411:494-498 (2001)). Furthermore, suppression in mammalian cells occurred at the RNA level and was specific for the targeted genes, with a strong correlation between RNA and protein suppression (Caplen, N. et al., Proc. Natl. Acad. Sci. USA 98:9746-9747 (2001)). In addition, it was shown that a wide variety of cell lines, including HeLa S3, COS7, 293, NIH/3T3, A549, HT-29, CHO-KI and MCF-7 cells, are susceptible to some level of siRNA silencing (Brown, D. et al. TechNotes 9(1):1-7, available at http://www.ambion.com/techlib/tn/91/912.html (Sep. 1, 2002)).

RNAi reagents can be readily prepared according to procedures known in the art. Structural characteristics of effective siRNA molecules have been identified. Elshabir, S. M. et al. Nature 411:494-498 (2001) and Elshabir, S. M. et al., EMBO 20:6877-6888 (2001). Accordingly, one of skill in the art would understand that a wide variety of different siRNA molecules may be used to target a specific gene or transcript. In certain embodiments, siRNA molecules according to the invention are 16-30 or 18-25 nucleotides in length, including each integer in between. In one embodiment, an siRNA is 21 nucleotides in length. In certain embodiments, siRNAs have 0-7 nucleotide 3′ overhangs or 0-4 nucleotide 5′ overhangs. In one embodiment, an siRNA molecule has a two nucleotide 3′ overhang. In one embodiment, an siRNA is 21 nucleotides in length with two nucleotide 3′ overhangs (i.e. they contain a 19 nucleotide complementary region between the sense and antisense strands). In certain embodiments, the overhangs are UU or dTdT 3′ overhangs. Generally, siRNA molecules are completely complementary to one strand of a target DNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases.

In one embodiment, siRNA target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, siRNA target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al. Nature 411:494-498 (2001); Elshabir, S. et al EMBO J. 20:6877-6888 (2001)). In addition, potential target sites may be compared to an appropriate genome database, such as BLASTN 2.0.5, available on the NCBI server at www.ncbi.nlm, and potential target sequences with significant homology to other coding sequences eliminated.

Short hairpin RNAs may also be used to inhibit or knockdown gene or nucleic acid expression according to the invention. Short Hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. It has been established that such short hairpin RNA-mediated gene silencing (also termed SHAGging) works in a variety of normal and cancer cell lines, and in mammalian cells, including mouse and human cells. Paddison, P. et al., Genes Dev. 16:948-58 (2002). Furthermore, transgenic cell lines bearing chromosomal genes that code for engineered shRNAs have been generated. These cells are able to constitutively synthesize shRNAs, thereby facilitating long-lasting or constitutive gene silencing that may be passed on to progeny cells. Paddison, P. et al., Proc. Natl. Acad. Sci. USA 99:1443-1448 (2002).

ShRNAs contain a stem loop structure. In certain embodiments, they contain variable stem lengths, typically from 19 to 29 nucleotides in length, or any number in between. In certain embodiments, hairpins contain 19 to 21 nucleotide stems, while in other embodiments, hairpins contain 27 to 29 nucleotide stems. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In fact, in certain embodiments, shRNAs are designed to include one or several G-U pairings in the hairpin stem to stabilize hairpins during propagation in bacteria, for example. However, complementarity between the portion of the stem that binds to the target mRNA (antisense strand) and the mRNA is typically required, and even a single base pair mismatch is this region may abolish silencing. 5′ and 3′ overhangs are not required, since they do not appear to be critical for shRNA function, although they may be present (Paddison et al. (2002) Genes & Dev. 16(8):948-58).

d. Expression Constructs

In certain embodiments, an inhibitor is introduced to a cell in an expression construct. In certain embodiments, expression constructs are transiently present in a cell, while in other embodiments, they are stably integrated into a cellular genome. Furthermore, it is understood that due to the inherent degeneracy of the genetic code, other DNA sequences that encode substantially the same or a functionally equivalent amino acid sequence or variants thereof may be produced and these sequences may be used to express a given polypeptide.

Methods well known to those skilled in the art may be used to construct expression vectors containing sequences encoding a polynucleotide or polypeptide of interest, e.g., an inhibitor of RecBC-mediated homologous recombination, and appropriate transcriptional and translational control elements. These methods include in vitro recombinant DNA techniques, synthetic techniques, and in vivo genetic recombination. Such techniques are described, for example, in Sambrook et al., Molecular Cloning—A Laboratory Manual (3^{rd} Ed.), Vol. 1-3, Cold Spring Harbor Laboratory, Cold Spring Harbor, 2000 and Ausubel, F. M. et al. (1989) Current Protocols in Molecular Biology, John Wiley & Sons, New York. N.Y., including all current supplements. In one embodiment, expression constructs of the invention comprise polynucleotide sequences corresponding to a region of a RecB gene.

Regulatory sequences present in an expression vector include those non-translated regions of the vector, e.g., enhancers, promoters, 5′ and 3′ untranslated regions, which interact with host cellular proteins to carry out transcription and translation. Such elements may vary in their strength and specificity. Depending on the vector system and cell utilized, any number of suitable transcription and translation elements, including constitutive and inducible promoters, may be used.

In mammalian cells, promoters from mammalian genes or from mammalian viruses are generally preferred, and a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T., Proc. Natl. Acad. Sci. 81:3655-3659 (1984)). In addition, transcription enhancers, such as the Rous sarcoma virus (RSV) enhancer, may be used to increase expression in mammalian host cells.

Vectors of the invention are introduced into a cell by any means available in the art, including, for example, electroporation, microinjection, transfection, infection, lipofection, gene gun, and retrotransposition. Generally, a suitable method of introducing a vector into a cell is readily determined by one of skill in the art based upon the type of vector and the type of cell, and teachings widely available in the art.

In certain embodiments, the invention provides for the conditional expression of an inhibitor of DNA repair or replication. A variety of conditional expression systems are known and available in the art for use in both cells and animals, and the invention contemplates the use of any such conditional expression system to regulate the expression or activity of a polypeptide involved in DNA repair or replication. In one embodiment of the invention, for example, expression of a molecule can be placed under control of the REV-TET system. Components of this system and methods of using the system to control the expression of a gene are well-documented in the literature, and vectors expressing the tetracycline-controlled transactivator (tTA) or the reverse tTA (rtTA) are commercially available (e.g., pTet-Off, pTet-On and ptTA-2/3/4 vectors, Clontech, Palo Alto, Calif.). Such systems are described, for example, in U.S. Pat. No. 5,650,298, No. 6271348, No. 5922927, and related patents, which are incorporated by reference in their entirety.

In particular embodiments, inhibitors are provided to a cell using a viral or bacteriophage vector. A wide variety of viral expression systems are known and available in the art, all of which may be used according to the invention. In one illustrative embodiment, retroviruses provide a convenient and effective platform for gene delivery systems. A selected nucleotide sequence can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman, BioTechniques 7:980-990 (1989); Miller, A. D., Human Gene Therapy 1:5-14 (1990); Scarpa et al., Virology 180:849-852 (1991); Burns et al., Proc. Natl. Acad. Sci. USA 90:8033-8037 (1993); and Boris-Lawrie and Temin, Cur. Opin. Genet. Develop. 3:102-109 (1993).

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham, J. Virol. 57:267-274 (1986); Bett et al., J. Virol. 67:5911-5921 (1993); Mittereder et al., Human Gene Therapy 5:717-729 (1994); Seth et al., J. Virol. 68:933-940 (1994); Barr et al., Gene Therapy 1:51-58 (1994); Berkner, K. L., BioTechniques 6:616-629 (1988); and Rich et al., Human Gene Therapy 4:461-476 (1993)).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., Molec. Cell. Biol. 8:3988-3996 (1988); Vincent et al., Vaccines 90 (Cold Spring Harbor Laboratory Press (1990)); Carter, B. J., Current Opinion in Biotechnology 3:533-539 (1992); Muzyczka, N., Current Topics in Microbiol. and Immunol. 158:97-129 (1992); Kotin, R. M., Human Gene Therapy 5:793-801 (1994); Shelling and Smith, Gene Therapy 1:165-169 (1994); and Zhou et al., J. Exp. Med. 179:1867-1875 (1994).

Additional viral vectors useful for delivering the polynucleotides encoding polypeptides of the present invention by gene transfer include those derived from the pox family of viruses, such as vaccinia virus and avian poxvirus. In addition, the invention contemplated the use of lentiviruses.

Additional illustrative information on these and other known viral-based delivery systems can be found, for example, in Fisher-Hoch et al., Proc. Natl. Acad. Sci. USA 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434 (1991); Kolls et al., Proc. Natl. Acad. Sci. USA 91:215-219 (1994); Kass-Eisler et al., Proc. Natl. Acad. Sci. USA 90:11498-11502 (1993); Guzman et al., Circulation 88:2838-2848 (1993); and Guzman et al., Cir. Res. 73:1202-1207 (1993).

In certain related embodiments, the present invention contemplates inhibiting the activity of a polypeptide involved in DNA repair or replication via gene knockout or knockdown. Methods of gene knockout are widely known and available in the art, and methods of constructing and using knockout and knockdown targeting vectors are known and described, e.g., in Gene Targeting, A Practical Approach, ed. Joyner, A. L., Oxford University Press (2000).

In another embodiment, the invention contemplated inhibiting the activity of a polypeptide component of a DNA repair or replication pathway by gene inversion. Accordingly, in certain embodiments, vectors of the invention comprise two recombinase recognition sites. Preferably, these recombinase recognition sites flank a sequence corresponding to a region of a gene encoding a polypeptide involved in DNA repair or replication. In preferred embodiments, the recombinase recognition sites are positioned to direct recombinase-mediated deletion of this sequence.

Suitable recombinase sites include FRT sites and loxP sites, which are recognized by the flp and cre recombinases, respectively (See U.S. Pat. No. 6,080,576, No. 5,434,066, and No. 4,959,317). The Cre-loxP and Flp-FRT recombinase systems are comprised of two basic elements: the recombinase enzyme and a small sequence of DNA that is specifically recognized by the particular recombinase. Both systems are capable of mediating the deletion, insertion, inversion, or translocation of associated DNA, depending on the orientation and location of the target sites. Recombinase systems are disclosed in U.S. Pat. No. 6,080,576, No. 5,434,066, and No. 4,959,317, and methods of using recombinase systems for gene disruption or replacement are provided in Joyner, A. L., Stricklett, P. K. and Torres, R. M. and Kuhn, R. IN LABORATORY PROTOCOLS FOR CONDITIONAL GENE TARGETING (1997), Oxford University Press, New York.

Representative minimal target sites for Cre and Flp are each 34 base pairs in length and are known in the art. The orientation of two target sites relative to each other on a segment of DNA directs the type of modification catalyzed by the recombinase: directly orientated sites lead to excision of intervening DNA, while inverted sites cause inversion of intervening DNA. In certain embodiments, mutated recombinase sites may be used to make recombination events irreversible. For example, each recombinase target site may contain a different mutation that does not significantly inhibit recombination efficiency when alone, but nearly inactivates a recombinase site when both mutations are present. After recombination, the regenerated recombinase site will contain both mutations, and subsequent recombination will be significantly inhibited. Recombinases useful in the present invention include, but are not limited to, Cre and Flp, and functional variants thereof, including, for example, FlpL, which contains an F70L mutation, and Flpe, which contains P2S, L33S, Y108N, and S294P mutations.

2. Polypeptide Inhibitors

In certain embodiments, inhibitors of the invention are polypeptides or related molecules, such as peptide mimetics. As used herein, the term polypeptide includes peptides and polypeptides of any length. Polypeptides can include natural amino acid residues, unnatural amino acid residues, or a combination thereof. These polypeptide inhibitors also target a component of a DNA repair or replication pathway and may act through a variety of different means. In certain embodiments, these inhibitors correspond to a portion of a polypeptide involved in DNA repair or replication or DNA repair.

In one embodiment, the activity of a polypeptide involved in DNA repair or replication is altered by overexpression of a dominant negative inhibitor of the polypeptide. Dominant negative inhibitors are typically mutant forms of a polypeptide, which reduce or block the activity of the wild type polypeptide, e.g., by competing for binding to a binding partner or substrate. In certain embodiments, dominant negative inhibitors are fragments of a wild type polypeptide. Examples of dominant negative mutants include, e.g., RecA mutants that are incapable of binding to single-stranded DNA, and specific functional or binding domains of RecBCD.

Polypeptide inhibitors further include variants, analogs, and derivatives. The term “analog” as used herein refers to a composition that retains the same structure or function (e.g., binding to a target) as a polypeptide or nucleic acid herein. Examples of analogs include peptidomimetics, peptide nucleic acids, small and large organic or inorganic compounds, as well as derivatives and variants of a polypeptide or nucleic acid herein. The term “derivative” or “variant” as used herein refers to a peptide or nucleic acid that differs from the naturally occurring polypeptide or nucleic acid by one or more amino acid or nucleic acid deletions, additions, substitutions or side-chain modifications. In certain embodiments, variants have at least 70%, at least 80% at least 90%, at least 95%, or at least 99% sequence identity to a wild type polypeptide. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally-occurring amino acid of similar character either in relation to polarity, side chain functionality or size.

Substitutions encompassed by the present invention may also be “non-conservative”, in which an amino acid residue which is present in a peptide is substituted with an amino acid having different properties, such as naturally occurring amino acid from a different group (e.g., substituting a charged or hydrophobic amino acid with alanine), or alternatively, in which a naturally-occurring amino acid is substituted with a non-conventional amino acid. Preferably, amino acid substitutions are conservative.

Amino acid substitutions are typically of single residues, but may be of multiple residues, either clustered or dispersed. Additions encompass the addition of one or more naturally occurring or non-conventional amino acid residues. Deletion encompasses the deletion of one or more amino acid residues.

In certain embodiments, peptide derivatives include peptides in which one or more of the amino acids has undergone side-chain modifications. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄. A variety of other modifications are known and available in the art. For example, in certain embodiments, peptide and peptidomimetics are modified for slower release or degradation, e.g., using D-amino acids or a PEG-terminus. A wide variety of unnatural amino acids can also be incorporated into polypeptides using available methods, e.g., using a cell or other translation system comprising an orthogonal synthetase, an orthogonal tRNA and a coding nucleic acid comprising a selector codon. For a review of this technology, see, e.g., Wang and Schultz “Expanding the Genetic Code,” Angewandte Chemie Int. Ed., 44(1):34-66 (2005), Xie and Schultz, “An Expanding Genetic Code,” Methods 36(3):227-238 (2005); Xie and Schultz, “Adding Amino Acids to the Genetic Repertoire,” Curr. Opinion in Chemical Biology 9(6):548-554; and Wang et al., “Expanding the Genetic Code,” Annu. Rev. Biophys. Biomol. Struct., epub Jan. 13, 2006; the contents of which are each incorporated by reference in their entirety. Unnatural amino acids can be used to alter protein function, half-life, uptake, reactivity to secondary molecules, or the like.

The present invention also encompasses all types of peptide mimetics (“peptidomimetics”). Peptidomimetics refer to molecules that mimic one or more aspects of a polypeptide structure. Specific examples of three types of peptide mimetics contemplated by the invention include: type I mimetics; which are amide bond mimetics and include transition state isosteres, amide backbone isosteres, β-strand mimetics and β-turn mimetics; type II mimetics, which are functional mimetics that produce the same function but do not bind at the same place in the receptor; and type III mimetics, which are non-peptide topographical mimetics that mimic the binding interactions of peptides.

Synthesis of polypeptides and derivatives and analogs thereof is known by those skilled in the art. In certain embodiments, it is preferable that a peptide or a peptidomimetic of the present inventions fit within the substrate binding site. Therefore, in certain embodiments, a peptide or peptidomimetic of the present invention is less than about 60 Angstroms, less than about 45 Angstroms, less than about 30 Angstroms, or less than about 15 Angstroms.

In a further embodiment, an inhibitor of the present invention is an evolved phage peptide ligand or another phage-derived protein. A variety of phage display methods for identifying peptides and polypeptides that bind to a target are known and available in the art. (see, e.g., Clackson, T. and Lowman, Phage Display: A Practical Approach (The Practical Approach Series, 266).

a. Antibody Inhibitors

Inhibitors of the present invention further include antibodies, or antigen-binding fragments thereof, specific for polypeptides associated with DNA repair or replication. In particular embodiments, an inhibitor of the present invention is an antibody or antigen-binding fragment thereof that specifically binds to RecA, RecB, PriA, Ku86, Ku70, or DNA-PK.

An antibody, or antigen-binding fragment thereof, is said to “specifically bind,” “immunologically bind,” and/or is “immunologically reactive” to a polypeptide if it reacts at a detectable level (within, for example, an ELISA assay) with the polypeptide, and does not react detectably with unrelated polypeptides under similar conditions. Antibodies are considered to specifically bind to a target polypeptide when the binding affinity is at least 1×10⁻⁷ M or, preferably, at least 1×10⁻⁸ M. Antibodies of the invention include, but are not limited to, monoclonal antibodies, chimeric antibodies, humanized antibodies, Primatized® antibodies, single chains, Fab fragments and scFv fragments.

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies via conventional techniques known in the art, or via transfection of antibody genes into suitable bacterial or mammalian cell hosts, in order to allow for the production of recombinant antibodies. Monoclonal antibodies specific for an antigenic polypeptide of interest may be prepared, for example, using the technique of Kohler and Milstein, Eur. J. Immunol. 6:511-519 (1976), and improvements thereto.

The Fab or F(ab′)₂ fragments may be wholly animal or human derived, or they may be in chimeric form, such that the constant domains are derived from the constant regions of human immunoglobulins and the variable regions are derived from the parent murine MAb. Alternatively, the Fv, Fab, or F(ab′)₂ may be humanized, so that only the complementarity determining regions (CDR) are derived from an animal MAb, and the constant domains and the framework regions of the variable regions are of human origin. These chimeric and humanized fragments are less immunogenic than their wholly animal counterparts, and thus more suitable for in vivo use, especially over prolonged periods. Methods of making chimeric and humanized antibodies are well known in the art, (See, e.g., U.S. Pat. No. 4,816,567, International Application No. WO84/03712, respectively).

In certain embodiments, an antibody inhibitor of the present invention is an intrabody. “Intracellular antibodies” or “intrabodies” are single-chain antibodies derived from a parent monoclonal antibody in which the variable domains of the light and heavy chains are joined together by a flexible peptide linker. The resulting recombinant gene product retains the ability of the parent antibody to bind to and neutralize the target antigen. As used herein, intrabodies encompass monoclonals, single chain antibodies, V regions, and the like, as long as they bind to a target protein. The entire intrabody sequence can be encoded on an expression plasmid, and the plasmid can be transfected into cells, leading to intracellular expression of the intrabody protein and neutralization of its intracellular protein antigen. Intrabodies are reviewed and described in a variety of articles, including Marasco, W. A., Gene Ther. 4: 11-15 (1997) and Persic, L., et al., Gene 187: 1-8 (1997).

Intracellular antibodies have found various applications, not only as research reagents but even more as therapeutic molecules. Initially, antibodies were microinjected into the cytoplasm of cells to block specifically the activity of cellular proteins. Recent advances in antibody engineering technology has led to two important developments: (1) the antigen binding domains of monoclonal antibodies can be expressed in recombinant form, e.g. as single-chain Fv fragments or Fab fragments; and (2) by using suitable expression systems, these fragments can be expressed in a variety of different cells, including mammalian cells.

By providing different signal sequences, recombinant antibodies can be directed to different subcellular compartments. The attachment of a hydrophobic mammalian leader sequence to the N-terminus of the antibody fragment results in the secretion of the molecule to the extracellular environment. Addition of an ER retention signal (e.g., KDEL) to the C-terminus of antibody fragments containing a leader sequence leads to retention in the lumen of the endoplasmatic reticulum. Using a C-terminal type 2 transmembrane domain instead of an ER retention signal, the antibody fragment is directed to the plasma membrane, resulting in cell surface display. Furthermore, antibody fragments can be directed to mitochondria by using a mitochondrial leader sequence, e.g. the leader sequence of cytochrome C oxidase subunit VIII. Cytoplasmic expression is achieved by expression of the antibody fragments without any signal or leader sequences. These fragments can be imported into the nucleus by fusing a nuclear localization sequence, e.g. from the SV40 large T antigen (PKKKRKV), to either the N- or C-terminus.

An important issue of intracellular expression of antibody fragments is the assembly of functional molecules. Since the antibody domains are stabilized by intradomain disulphide bonds, an oxidizing milieu is necessary for functional assembly. This environment is found for example in the secretory pathway and antibodies directed to this pathway are generally assembled into functional antibody molecules. Formation of disulphide bonds was also reported for scFv fragments directed to mitochondria. In contrast, the cytoplasm and the nucleus have a reducing milieu and the formation of functional molecules in this compartment is either abolished or reduced. Nevertheless, various groups have reported the functional expression of recombinant antibody fragments in the cytoplasm and nucleus. The engineering of functional antibody fragments without cysteine residues that has recently been shown will help to improve functional expression of antibody fragments in these compartments.

It has been demonstrated that intrabodies are capable of effectively neutralizing gp120, the glycoprotein that studs the outer surface of HIV. One such intrabody is sFv105, a modified version of a human monoclonal antibody that recognizes the CD4 binding site of gp120, which plays a critical role in the infection process. Stripped of its glycoprotein coat, the “naked” HIV is unable to infect new cells. In addition, by blocking the expression of gp120 on the surface of infected CD4 cells, sFv105 inhibits the formation of syncytia, the lethal cell clusters that trigger widespread cell death.

3. Small Molecule Inhibitors

Inhibitors of the present invention further include large or small inorganic or organic molecules. In certain embodiments, inhibitors are small organic molecules, or derivatives or analogs thereof. In preferred embodiments, inhibitors of the present invention are able to permeate or enter a microorganism or cell. Thus, in one embodiment, preferred inhibitors are bacterial permeable. In other embodiments, inhibitors are able to enter a microorganims or cell by passive diffusion or active transport, including, e.g., receptor-mediated uptake.

In certain embodiments, an inhibitor includes a protecting group. The term “protecting group” refers to chemical moieties that block at least some reactive moieties and prevent such groups from participating in chemical reactions until the protective group is removed (or “cleaved”). Examples of blocking/protecting groups are described, e.g., in Greene and Wuts, Protective Groups in Organic Synthesis, 3rd Ed., John Wiley & Sons, New York, N.Y., 1999.

Any of the inhibitors may possess one or more chiral centers and each center may exist in the R or S configuration. Inhibitors of the present invention include all diastereomeric, enantiomeric, and epimeric forms as well as mixtures thereof. Stereoisomers may be obtained, if desired, by methods known in the art as, for example, the separation of stereoisomers by chiral chromatographic columns. Inhibitors further include of N-oxides, crystalline forms (also known as polymorphs), and pharmaceutically acceptable salts, as well as active metabolites of any inhibitor. All tautomers are included within the scope of the inhibitors presented herein. In addition, the inhibitors described herein can exist in unsolvated as well as solvated forms with pharmaceutically acceptable solvents such as water, ethanol, and the like. The solvated forms of the inhibitors presented herein are also included within the present invention.

In a particular embodiment, a small molecule inhibitor binds to RecB, RecA, or PriA. Specific inhibitors of the present invention bind at or near a functional site identified in any of these molecules, including, e.g., a chi cutting site, helicase domain, nuclease domain, tunnel region of RecB, a helicase, primase, or ATPase domain of PriA, or a DNA binding domain of RecA.

B. Pharmaceutical Compositions and Kits

The present invention further includes formulations of inhibitors of DNA repair or replication. Formulations are typically adapted for particular uses and include, e.g., pharmaceutical compositions suitable for administration to a patient, i.e., physiologically compatible. Accordingly, compositions of the inhibitors will often further comprise one or more buffers or carriers. In any of the compositions or formulations herein, the inhibitor can be formulated, e.g., as a salt, a drug, a prodrug, or a metabolite.

In addition, compositions of the present invention may comprise a pharmaceutically effective buffer or carrier. As used herein, a “pharmaceutical acceptable carrier” is a pharmaceutically acceptable solvent, suspending agent or vehicle for delivering an inhibitor of the present invention to a microorganism, animal or human. The carrier may be, for example, gaseous, liquid or solid and is selected with the planned manner of administration in mind.

General examples of carriers include buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

Examples of pharmaceutically acceptable carriers for oral pharmaceutical formulations include lactose, sucrose, gelatin, agar and bulk powders. In certain embodiments, pharmaceutical compositions of the present invention are formulated as tablets or capsules for oral administration. Such tablets or capsules may be formulated for specific release characteristics, e.g., extended release capsules. In particular embodiments, wherein a composition of the invention comprises both an inhibitor of DNA repair or replication and another antimicrobial or cytotoxic agent, the composition may be formulated as a mixture or in layers, e.g., the antimicrobial or cytotoxic agent may be encapsulated by the inhibitor or vice versa. In another embodiment, pharmaceutical compositions of the present invention comprises two or more inhibitors of the present invention.

Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also preferred carriers.

Examples of pharmaceutically acceptable carriers for topical formulations include: ointments, cream, suspensions, lotions, powder, solutions, pastes, gels, spray, aerosol or oil. Alternately, a formulation may comprise a transdermal patch or dressing such as a bandage impregnated with an active ingredient (e.g., inhibitor and/or second therapeutic agent) and optionally one or more carriers or diluents. The topical formulations may include a compound that enhances absorption or penetration of the active ingredient through the skin or other affected areas. Examples of such dermal penetration enhancers include dimethylsulfoxide and related analogues. In certain embodiments, to be administered in the form of a transdermal delivery system, the dosage administration will be continuous rather than intermittent throughout the dosage regimen.

Formulations suitable for parenteral administration include aqueous and non-aqueous formulations isotonic with the blood of the intended recipient; and aqueous and non-aqueous sterile suspensions which may include suspending systems designed to target the compound to blood components of one or more organs. The formulations may be presented in unit-dose or multi-dose sealed containers, for example, ampoules or vials. Extemporaneous injection solutions and suspensions may be prepared from sterile powders, granules and tablets of the kind previously described. Parenteral and intravenous formulation may include minerals and other materials to make them compatible with the type of injection or delivery system chosen.

Commonly used pharmaceutically acceptable carriers for parenteral administration includes, water, a suitable oil, saline, aqueous dextrose (glucose), or related sugar solutions and glycols such as propylene glycol or polyethylene glycols. Solutions for parenteral administration preferably contain a water soluble salt of the active ingredient, suitable stabilizing agents and, if necessary, buffer substances, antioxidizing agents, such as sodium bisulfite, sodium sulfite, or ascorbic acid, either alone or combined, are suitable stabilizing agents. Citric acid salts and sodium EDTA may also be used as carriers. In addition, parenteral solutions may contain preservatives, such as benzalkonium chloride, methyl- or propyl-paraben, or chlorobutanol. Suitable pharmaceutical carriers are described in Remington, cited above.

The present invention additionally contemplates inhibitors formulated for veterinary administration by methods conventional in the art.

The inhibitors described herein can also be formulated for industrial applications with, for example, a cleaning product, such as soap, laundry detergent, shampoo, dishwashing soap, toothpaste, and other house cleaning detergents.

The compositions and pharmaceutical formulation herein can be administered to an organism by any means known in the art. Routes for administering the compositions and pharmaceutical formulations herein to an animal, such as a human, include parenterally, intravenously, intramuscularly, orally, by inhalation, topically, vaginally, rectally, nasally, buccally, transdermally, as eye drops, or by an implanted reservoir external pump or catheter.

Injectable formulations can be prepared in conventional forms, either as liquid solutions or suspensions; as solid forms suitable for solubilization or suspension in liquid prior to injection; or as emulsions. Preferably, sterile injectable suspensions are formulated according to techniques known in the art using suitable pharmaceutically acceptable carriers and other optional components, as described above.

Parenteral administration may be carried out in any number of ways, but it is preferred that a syringe, catheter, or similar device, be used to effect parenteral administration of the formulations described herein. The formulation may be injected systemically such that the active agent travels substantially throughout the entire bloodstream.

Also, the formulation may also be injected locally to a target site, e.g., injected to a specific portion of the body for which inhibition of a pathway using DNA repair or replication is desired. An advantage of local administration via injection is that it limits or avoids exposure of the entire body to the active agent(s) (e.g., inhibitors and/or other therapeutic agents). It must be noted that in the present context, the term local administration includes regional administration, e.g., administration of a formulation directed to a portion of the body through delivery to a blood vessel serving that body zone. Local delivery may be direct, e.g., intratumoral. Local delivery may also be nearly direct, i.e., intralesional or intraperitoneal, that is, to an area that is sufficiently close to a tumor or site of infection so that the inhibitor exhibits the desired pharmacological activity. Thus, when local delivery is desired, the pharmaceutical formulations are preferably delivered intralesionally, intratumorally, or intraperitoneally.

In certain embodiments, the pharmaceutical compositions are in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet, or it can be the appropriate number of any of these packaged forms.

Pharmaceutical compositions of the present invention will typically comprise an amount of inhibitor that is sufficient to achieve a therapeutic or prophylactic effect upon administration to a patient at a prescribed dosage. The actual effective amount will depend upon the condition being treated, the route of administration, the drug treatment used to treat the condition, and the medical history of the patient. Determination of the effective amount is well within the capabilities of those skilled in the art. The effective amount for use in humans can be determined from animal models. For example, a dose for humans can be formulated to achieve circulating concentrations that have been found to be effective in animals. The effective amount of an inhibitor can vary if the inhibitor is coformulated with another therapeutic agent (e.g., antimicrobial or cytotoxic agent or compound, such as an antibiotic, an antineoplastic agent, an antiviral agent, an antiprotozoan agent, etc.).

In particular embodiments, an effective amount of an active ingredient (e.g., an inhibitor or second therapeutic agent) is from about 0.0001 mg to about 500 mg active agent per kilogram body weight of a patient, more preferably from about 0.001 to about 250 mg active agent per kilogram body weight of the patient, still more preferably from about 0.01 mg to about 100 mg active agent per kilogram body weight of the patient, yet still more preferably from about 0.5 mg to about 50 mg active agent per kilogram body weight of the patient, and most preferably from about 1 mg to about 15 mg active agent per kilogram body weight of the patient. In terms of weight percentage, a pharmaceutical formulation of an active agent (e.g., an inhibitor or second therapeutic agent) preferably comprises of an amount from about 0.0001 wt. % to about 10 wt. %, more preferably from about 0.001 wt. % to about 1 wt. %, and more preferably from about 0.01 wt. % to about 0.5 wt. %.

In certain embodiments, compositions and formulations of the present invention that include an inhibitor of DNA repair or replication also include one or more additional therapeutic agent(s), preferably an antimicrobial or cytotoxic compound or agent, such as, e.g., an antibiotic, an antiviral agent, an antifungal agent, an antiprotozoan agent, and/or an antineoplastic agent. In addition, a composition comprising an inhibitor of DNA repair or replication may be administered in combination with one or more additional therapeutic agents.

In various embodiments, an inhibitor of the DNA repair and replication is co-formulated with an additional therapeutic agent. An inhibitor may be provided to a microorganism, cell or patient before, at the same time as, of after an additional therapeutic agent is provided to the microorganism, cell or patient.

In certain embodiments, a composition of the present invention further comprises or is administered in combination with an antibiotic. Examples of antibiotics that may be coformulated or administered with an inhibitor of DNA repair or replication include aminoglycosides, carbapenems, cephalosporins, cephems, glycopeptides, fluoroquinolones/quinolones, macrolides, oxazolidinones, penicillins, streptogramins, sulfonamides, and tetracyclines. In various related embodiments, a composition of the present invention further comprises or is administered in combination with an anti-tumor, anti-viral and/or an anti-malarial agent. Similarly, in particular embodiments, a composition of the present invention comprises two or more inhibitors of the present invention, alone, or in combination with an additional therapeutic agent, such as an antibiotic, anti-tumor, anti-viral, or anti-malarial agent.

Aminoglycosides are a group of antibiotics found to be effective against gram-negative bacteria. Aminoglycosides are used to treat complicated urinary tract infections, septicemia, peritonitis and other severe intra-abdominal infections, severe pelvic inflammatory disease, endocarditis, mycobacterium infections, neonatal sepsis, and various ocular infections. They are also frequently used in combination with penicillins and cephalosporins to treat both gram-positive and gram-negative bacteria. Examples of aminoglycosides include amikacin, gentamycin, tobramycin, netromycin, streptomycin, kanamycin, paromomycin, and neomycin.

Carbapenems are a class of broad-spectrum antibiotics that are used to fight gram-positive, gram-negative, and anaerobic microorganisms. Carbapenems are available for intravenous administration, and as such are used for serious infections which oral drugs are unable to adequately address. For example, carbapenems are often used to treat serious single or mixed bacterial infections, such as lower respiratory tract infections, urinary tract infections, intra-abdominal infections, gynecological and postpartum infections, septicemia, bone and joint infections, skin and skin structure infections, and meningitis. Examples of carbapenems include imipenem/cilastatin sodium, meropenem, ertapenem, and panipenem/betamipron.

Cephalosporins and cephems are broad spectrum antibiotics used to treat gram-positive, gram-negative, and spirochaetal infections. Cephems are considered the next generation cephalosporins with newer drugs being stronger against gram negative and older drugs better against gram-positive. Cephalosporins and cephems are commonly substituted for penicillin allergies and can be used to treat common urinary tract infections and upper respiratory infections (e.g., pharyngitis and tonsillitis). Cephalosporins and cephems are also used to treat otitis media, some skin infections, bronchitis, lower respiratory infections (pneumonia), and bone infection (certain members), and are a preferred antibiotic for surgical prophylaxis. Examples of cephalosporins include cefixime, cefpodoxime, ceftibuten, cefdinir, cefaclor, cefprozil, loracarbef, cefadroxil, cephalexin, and cephradineze. Examples of cephems include cefepime, cefpirome, cefataxidime pentahydrate, ceftazidime, ceftriaxone, ceftazidime, cefotaxime, cefteram, cefotiam, cefuroxime, cefamandole, cefuroxime axetil, cefotetan, cefazolin sodium, cefazolin, cefalexin.

Fluroquinolones/quinolones are antibiotics used to treat gram-negative infections, though some newer agents have activity against gram-positive bacteria and anaerobes. Fluroquinolones/quinolones are often used to treat conditions such as urinary tract infections, sexually transmitted diseases (e.g., gonorrhea, chlamydial urethritis/cervicitis, pelvic inflammatory disease), gram-negative gastrointestinal infections, soft tissue infections, ophthalmic infections, dermatological infections, surgical site infections, sinusitis, and respiratory tract infections (e.g., bronchitis, pneumonia, and tuberculosis). Fluroquinolones/quinolones are also used in combination with other antibiotics to treat conditions, such as multi-drug resistant tuberculosis, neutropenic cancer patients with fever, and potentially anthrax. Examples of fluoroquinolones/quinolones include ciproflaxacin, levofloxacin, ofloxacin, cinoxacin, nalidixic acid, gatifloxacin, norfloxacin, lomefloxacin, trovafloxacin, moxifloxacin, sparfloxacin, gemifloxacin, and pazufloxacin. Other quinolones have been recently described, including the nonfluorinated quinolones, PGE 926932 and PGE 9509924 (Jones, M. E. et al., Antimicrob Agents Chemother. 46:1651-7 (2002)) and ciprofloxacin dimers (Gould, K. A., et al., Antimicrob Agents Chemother. 48:2108-15 (2004)). However, certain fluoroquinolones are not widely available due to side effects. For example, sparfloxacin is associated with a high incidence of photosensitivity, grepafloxacin is associated with QTc prolongation, and loefloxacin is associated with a high incidence of photosensitivity.

Glycopeptides represent antibiotics that are used to treat bacteria that are resistant to other antibiotics, such as methicillin-resistant staphylococcus aureus (MRSA). They are also be used for patients who are allergic to penicillin. Examples of glycopeptides include vancomycin, teicoplanin, and daptomycin.

Macrolides are broad-spectrum antibiotics and are an important alternative to penicillins and cephalosporins. Macrolides are often used to treat respiratory tract infections (e.g., otitis media, chronic sinusitis, bronchitis, pharyngitis, pneumonia, tonsillitis, and strep throat), sexually transmitted diseases (e.g., infections of the cervix and urinary tract, genital ulcer disease in men, syphilis), and opportunistic infections (e.g., pneumonia and mycobacterium avium complex (MAC) infection). Examples of macrolides include erythromycin, clarithromycin, azithromycin, axithromycin, dirithromycin, troleandomycin, oleandomycin, roxithromycin, and telitrhomycin.

Oxazolidinones are commonly administered to treat gram-positive infections. Carbapenems are used to treat gram-positive, gram-negative, and/or anaerobes. Oxazolidinones are commonly used as an alternative to other antibiotic classes for bacteria that have developed resistance. Examples of oxazolidinones include linezolid.

Penicillins are broad spectrum used to treat gram-positive, gram-negative, and spirochaetal infections. Conditions that are often treated with penicillins include pneumococcal and meningococcal meningitis, dermatological infections, ear infections, respiratory infections, urinary tract infections, acute sinusitis, pneumonia, and lyme disease. Examples of penicillins include penicillin, amoxicillin, amoxicillin-clavulanate, ampicillin, ticarcillin, piperacillin-tazobactam, carbenicillin, piperacillin, mezocillin, benzathin penicillin G, penicillin V potassium, methicillin, nafcillin, oxacillin, cloxacillin, and dicloxacillin.

Streptogramins are antibiotics developed in response to bacterial resistance that diminished effectiveness of existing antibiotics. Streptogramins are a very small class of drugs and are currently very expensive. Examples of streptogramins include quinupristin/dafopristin and pristinamycin.

Sulphonamides are broad-spectrum antibiotics that have had reduced usage due to harmful adverse events and an increase in bacterial resistance to them. Suphonamides are commonly used to treat recurrent attacks of rheumatic fever, urinary tract infections, prevention of infections of the throat and chest, traveler's diarrhea, whooping cough, meningococcal disease, sexually transmitted diseases, toxoplasmosis, and rhinitis. Examples of sulfonamides include co-trimoxazole, sulfamethoxazole trimethoprim, sulfadiazine, sulfadoxine, and trimethoprim.

Tetracyclines are broad-spectrum antibiotics that are often used to treat gram-positive, gram-negative, and/or spirochaetal infections. Tetracyclines are often used to treat mixed infections, such as chronic bronchitis and peritonitis, urinary tract infections, rickets, chlamydia, gonorrhea, lyme disease, and periodontal disease. Tetracyclines are an alternative therapy to penicillin in syphilis treatment and are also used to treat acne and anthrax. Examples of tetracyclines include tetracycline, demeclocycline, minocycline, and doxycycline.

Other antibiotics contemplated herein (some of which may be redundant with the list above) include abrifam; acrofloxacin; aptecin, amoxicillin plus clavulonic acid; amikacin; apalcillin; apramycin; astromicin; arbekacin; aspoxicillin; azidozillin; azithromycin; azlocillin; aztreonam; bacitracin; benzathine penicillin; benzylpenicillin; clarithromycin, carbencillin; cefaclor; cefadroxil; cefalexin; cefamandole; cefaparin; cefatrizine; cefazolin; cefbuperazone; cefcapene; cefdinir; cefditoren; cefepime; cefetamet; cefixime; cefmetazole; cefminox; cefoperazone; ceforanide; cefotaxime; cefotetan; cefotiam; cefoxitin; cefpimizole; cefpiramide; cefpodoxime; cefprozil; cefradine; cefroxadine; cefsulodin; ceftazidime; ceftriaxone; cefuroxime; cephalexin; chloramphenicol; chlortetracycline; ciclacillin; cinoxacin; ciprofloxacinfloxacin; clarithromycin; clemizole penicillin; cleocin, cleocin-T, clindamycin; cloxacillin; corifam; daptomycin; daptomycin; demeclocycline; desquinolone; dibekacin; dicloxacillin; dirithromycin; doxycycline; enoxacin; epicillin; erthromycin; ethambutol; gemifloxacin; fenampicin; finamicina; fleroxacin; flomoxef; flucloxacillin; flumequine; flurithromycin; fosfomycin; fosmidomycin; fusidic acid; gatifloxacin; gemifloxaxin; gentamicin; imipenem; imipenem plus cilistatin combination; isepamicin; isoniazid; josamycin; kanamycin; kasugamycin; kitasamycin; kalrifam, latamoxef; levofloxacin, levofloxacin; lincomycin; linezolid; lomefloxacin; loracarbaf; lymecycline; mecillinam; meropenem; methacycline; methicillin; metronidazole; meziocillin; midecamycin; minocycline; miokamycin; moxifloxacin; nafcillin; nafcillin; nalidixic acid; neomycin; netilmicin; norfloxacin; novobiocin; oflaxacin; oleandomycin; oxacillin; oxolinic acid; oxytetracycline; paromycin; pazufloxacin; pefloxacin; penicillin g; penicillin v; phenethicillin; phenoxymethyl penicillin; pipemidic acid; piperacillin; piperacillin and tazobactam combination; piromidic acid; procaine penicillin; propicillin; pyrimethamine; rifadin; rifabutin; rifamide; rifampin; rifamycin sv; rifapentene; rifomycin; rimactane, rofact; rokitamycin; rolitetracycline; roxithromycin; rufloxacin; sitafloxacin; sparfloxacin; spectinomycin; spiramycin; sulfadiazine; sulfadoxine; sulfamethoxazole; sisomicin; streptomycin; sulfamethoxazole; sulfisoxazole; quinupristan-dalfopristan; teicoplanin; telithromycin; temocillin; gatifloxacin; tetracycline; tetroxoprim; telithromycin; thiamphenicol; ticarcillin; tigecycline; tobramycin; tosufloxacin; trimethoprim; trimetrexate; trovafloxacin; vancomycin; verdamicin; azithromycin; and linezolid.

In certain embodiments, an inhibitor of the present invention is used to treat a microorganism or cell resistant to or in combination with a drug that asserts its effect by causing DNA damage or inhibiting DNA replication or repair. Similarly, an inhibitor of the present invention is also used to sensitize cells to a drug that asserts its effect by causing DNA damage or inhibiting DNA replication or repair. A variety of antimicrobial and chemotherapeutic agents are known to involve such mechanisms. For example, sulphonamides interfere with the use of folic acid and inhibit bacterial replication. Also, as described herein, fluoroquinolones inhibit DNA replication by targeting DNA gyrase and topoisomerase IV. In addition, certain DNA damaging agents, e.g., trimethorprim and aminopterin, cause DNA damage associated with DNA sensitivity or “thymineless death.”

As described herein, in certain methods of the present invention, an inhibitor of a polypeptide associated with DNA repair or replication or fork repair is used to treat a drug-resistant microorganism or cell. A variety of drug-resistant microorganisms have been identified and are known in the art. For example, methicillin-resistant Staphylococcus aureus, vancomycin-resistant Enterococci, and fluoroquinolone-resistant Pseudomonas aeruginosa pose significant resistance problems. Resistance to fluoroquinolones has been reported in a variety of microorganisms, including methicillin-susceptible Staphylococcus aureus, Campylobacter jejuni/coli, Salmonella, Shigella, and E. coli. Resistance emerged first in species in which single mutations were sufficient to cause clinically important levels of resistance, e.g., Staphylococcus aureus and Pseudomonas aeruginosa (Emerg Infect Dis. 7:337-41 (2001)). Subsequently, resistance has emerged in bacteria such as Campylobacter jejuni, E. coli, and Neisseria gonorrhoeae, in which multiple mutations are generally observed in clinically important resistance.

One particular bacterial strain exhibiting a wide spectrum of resistance is S. pneumoniae. Resistance was 34% for penicillin, 20-30% for macrolides, 17% for tetracyclines, 36% for trimethoprim-sulfamethoxazole, 3% for fluoroquinoles and 22% multi-drug resistant (Doem, G. et al., Antimicrob Agents Chemother 45:1721-1729 (2004); Jacobs, M. R., Clin Infect Dis 35:565-569 (2002); Hoban, D. J. et al., Clin Infect Dis 32:581-583 (2001)). The incidence of fluoroquinolone-resistant pneumococci is currently low, but recently, cases of fluoroquinolone-resistant strains of S. pneumoniae have been observed.

Resistance is also becoming increasingly prevalent to a number of other widely-used antimicrobial agents, including, e.g., penicillin, erythromycin, levofloxacin and telithromycin. In addition, resistant strains of Pseudomonas prove a continuing problem, as the incidence of P. aeruginosa infections in hospitals and other institutions is increasing, and currently accounting for over 10 percent of all hospital-acquired infections. Only a few antibiotics are currently considered effective against Pseudomonas, including various fluoroquinolones, gentamicin and imipenem, and even these antibiotics are not effective against all strains. For example, the futility of treating Pseudomonas infections with currently available antibiotic protocols is dramatically illustrated in cystic fibrosis patients, virtually all of whom eventually become infected with a drug-resistant strain that cannot be effectively treated.

A non-exhaustive list of examples of known drug resistance includes: ciprofloxacin resistant S. aureus, coagulase-negative Staph, E. faecalis, E. faecium, E. coli, K. oxytoca, K. pneumoniae, M. morganii, P. mirabilis, S. marcescens, Acinetobacter, and P. aeruginosa; levofloxacin resistant S. pneumoniae, S. pyogenes, S. agalactiae, Viridans group, E. coli, K. oxytoca, K. pneumoniae, M. morganii, P. mirabilis, S. marcenscens, Acinetobacter, and P. aeruginosa; sulfamethoxazole trimethoprim resistant E. coli, K. oxytoca, K. pneumoniae, M. Morganii, P. mirabilis, S. marcenscens, Acinetobacter, and P. aeruginosa; ampicillin resistant S. aureus, coagulase-negative staph, E. faecalis, E. faecium, and S. pneumoniae; oxacillin resistant S. aureus and coagulase-negative staph; penicillolin resistant S. pneumoniae and Virdans group; piperacillin-tazobactam resistant E. coli, K. oxytoca, K. pneumoniae, M. morganii, P. mirabilis, S. marcescens, Acinetobacter, and P. aeruginosa; cefapine resistant S. aureus, coagulase-negative staph, S. pneumoniae, E. coli, K. oxytoca, K. pneumoniae, M. morganii, P. mirabilis, S. marcescens, Acinobacter, and P. aeruginosa; cefotaxime resistant S. aureus, coagulase-negative staph, S. pneumoniae, E. coli, K. oxytoca, K. pneumoniae, M. morganii; P. mirabilis, S. marcenscens, Acinetobacter, and P. aeruginosa; ceftriaxone resistant S. aureus, coagulase-negative staph, S. pneumoniae, M. morganii, P. mirabilis, S. marcescens, Acinetobacter, and P. aeruginosa; gentamycin resistant S. aureus, coagulase-negative staph, E. faecalis, E. faecium, E. coli, K. oxytoca, K. pneumoniae, M. morganii, P. mirabilis, S. marcenscens, Acinobacter, and P. aeruginosa; clarithromycin resistant S. pneumoniae, S. pyogenes, S. agalactiae, and Virdans group; erythromycin resistant S. pneumoniae, S. pyogenes, and S. agalactiae, and Virdans group; teicoplanin resistant E. faecium; vancomycin resistant E. faecalis and E. faecium; and imipenem resistant Acinobacter and P. aeruginosa.

In certain embodiments, a composition of the present invention further comprises or is administered in combination with an antifungal. A variety of different classes of antifungal agents exist. Example of antifungals include, but are not limited to, allymines and other non-azole ergosterol biosynthesis inhibitors, antimetabolites, azoles, glucan synthesis inhibitors, polyenes, and other miscellaneous systemic antifungals.

In certain embodiments, a composition of the present invention further comprises or is administered in combination with an antiviral agent. Examples of antiviral agents include, but are not limited to idoxuridine (IDU), which is used in topical therapy of herpes simplex keratoconjunctivitis; vidarabine (adenine arabinoside, ara-A), which is used, e.g., in the treatment of HSV infections; trifluridine (trifluorothymidine), a thymidine analog, which interferes with DNA synthesis and is effective in treating primary keratoconjunctivitis and recurrent keratitis caused by HSV-1 and HSV-2; acyclovir, which is a purine nucleoside analog with activity against herpes and cytomegalovirus (CMV); famciclovir, which is a pro-drug of the active antiviral penciclovir and is used to treat HSV-1, HSV-2, VZV, EBV, CMV, and HBV; penciclovir, a guanosine analog that inhibits HSV-1 and HSV-2 viral DNA polymerase; valacyclovir; ganciclovir which is used against all herpes viruses, including CMV, as well as HIV and CMV retinitis; foscarnet, an organic analog of inorganic pyrophosphate that inhibits virus-specific DNA polymerase and reverse transcriptase; ribavirin, a guanosine analog that inhibits the replication of many RNA and DNA viruses; amantadine and rimantadine, which are used primarily for influenza A prophylaxis and treatment, interfere with the development of immunity from the vaccine; cidofovir (cytosine; HPMPC), which is a nucleotide analog that has inhibitory in vitro activity against a broad spectrum of viruses, including HSV-1, HSV-2, VZV, CMV, EBV, adenovirus, human papillomavirus (HPV), and human polyomavirus, as well as oligonucleotides, immune globulins, such as hyperimmune CMV immunoglobulin and interferons.

In certain embodiments, a composition of the present invention further comprises or is administered in combination with an antineoplastic agent or chemotherapeutic compound. In particular embodiments, the antineoplastic agent is a DNA damaging agent, an agent that inhibits DNA replication, or a topoisomerase poison. Antracyclines, amsacrine and ellipticines are examples of intercalating agents that act as topoisomerase II poisons. Camptothecin and VM26 (teniposide) are representative DNA topoisomerase poisons that target DNA topoisomerase I and topoisomerase II, respectively. Camptothecin (CPT) compounds include various 20(S)-camptothecins, analogs of 20(S)camptothecin, and derivatives of 20(S)-camptothecin. Camptothecin, when used in the context of this invention, includes the plant alkaloid 20(S)-camptothecin, both substituted and unsubstituted camptothecins, and analogs thereof. Examples of camptothecin derivatives include, but are not limited to, 9-nitro-20(S)-camptothecin, 9-amino-20(S)-camptothecin, 9-methyl-camptothecin, 9-chlorocamptothecin, 9-flouro-camptothecin, 7-ethyl camptothecin, 10-methylcamptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as described in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate. Particular examples of 20(S)-camptothecins include 9-nitrocamptothecin, 9-aminocamptothecin, 10,11-methylendioxy-20(S)camptothecin, topotecan, irinotecan, 7-ethyl-10-hydroxy camptothecin, or another substituted camptothecin that is substituted at least one of the 7, 9, 10, 11, or 12 positions. These camptothecins may optionally be substituted.

Other examples of antineoplastic agents that may be coformulated or administered with an inhibitor of the present invention include: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine; hydrochloride; elsamitrucin; enloplatin; enpromate; epipropidine; epirubicin hydrochloride; erbulozole; esorubicin hydrochloride; estramustine; estramustine phosphate sodium; etanidazole; ethiodized oil; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; imofosine; interferon alpha-2a; interferon alpha-2b; interferon alpha-n1; interferon alpha-n3; interferon beta-Ia; interferon gamma-Ib; iproplatin; irinotecan hydrochloride; lanreotide acetate; letrozole; leuprolide acetate liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycinl; spirogermanium hydrochloride; spiromustine; spiroplatin; streptonigrin; streptozocin; strontium chloride sr 89; sulofenur; talisomycin; taxane; taxoid; tecogalan sodium; tegafur; teloxantrone hydrochloride; temoporfin; teniposide; teroxirone; testolactone; thiamiprine; thioguanine; thiotepa; tiazofurin; tirapazamine; topotecan hydrochloride; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride. Additional antineoplastic agents that are disclosed herein or known in the art are also contemplated by the present invention.

The present invention also includes kits comprising one or more inhibitors of DNA repair or replication. Kits may further comprise one or more additional therapeutic compounds (antimicrobial or cytotoxic agent or compound, e.g., an antimicrobial agent, such as an antibiotic, antifungal, or antiviral, antiprotozoan, or a cytotoxic agent, e.g., a chemotherapeutic agent).

Typically, kits of the present invention comprise one or more vials or containers, with one of said vials comprising an inhibitor of the present invention, as well as instructions for the use of the kit. For example, instructions can direct an individual as to the specific inhibitor to be used, dosages to be applied, frequency and duration of use, and methods of administration. Preferably, a vial comprises an inhibitor in a pharmaceutical formulation. In some embodiments, a kit comprises one or more vials of an inhibitor formulated for local or system administration. In certain embodiments, an additional vial comprises another therapeutic agent (e.g., an antibiotic, an antiviral, an antifungal, an antineoplastic, or an antiprotozoan medication). The inhibitor and the second therapeutic agent can be combined prior to administration or may be administered separately.

As noted, inhibitors of the present invention sensitize microorganisms and cells to antimicrobial and cytotoxic agents. Accordingly, the use of an inhibitor of the present invention permits the use of a lower dose of an antimicrobial or cytotoxic agent than necessary for a therapeutic or prophylactic effect in the absence of the inhibitor. Essentially, the use of an inhibitor of the present invention in combination with the other therapeutic agent or drug reduces the MIC for that therapeutic agent or drug.

Accordingly, the present invention permits the use of lower dosages of antimicrobial and cytotoxic agents, including, but not limited to, the antibiotics and chemotherapeutic agents described herein, than previously used or shown to be efficacious in the art. This offers clear advantages, in that it reduces side effects associated with higher dosages of active agents and reduces the cost associated with treatment with anitmicrobial and cytotoxic agents. Thus, in certain situations, cooadministration (including pre- or post-administration) with an inhibitor of the present invention permits the use of antimicrobial or cytotoxic agents that were previously unavailable or inadvisable for use in one or more patient populations due to potential side effects or high cost. This further permits the use of antimicrobial or cytotoxic agents for previously unprescribed usages, e.g., for indications that were not considered serious enough to risk any potential side effects and for indications that previously had less expensive alternative therapeutic options. In addition, the use of lower dosages facilitates the use of certain drugs in pediatric patients. For example, the use of fluoroquinolones, e.g., ciprofloxacin, is generally avoided in pediatric patients due to potential cartilage damage. The ability to use lower dosages in combination with an inhibitor of the present invention permits the use of such drugs in pediatric patents. Similarly, the present invention permits the use of drugs that were previously not used due to their having an undesirable toxicity profile at the dosage required for efficacy. Reducing the required dosage can reduce the toxicity profile to an acceptable level.

Therefore, in certain embodiments of kits and methods of the present invention, wherein said kit includes an additional active or therapeutic agent or said method involves administering an additional active or therapeutic agent, the kit or method comprises a lesser amount of the additional active agent or a lower unit dosage form of said additional active agent than previously used in the art.

The invention further includes methods of manufacturing and processes for producing an inhibitor of the present invention. In one embodiment, a process of producing a compound that enhances the sensitivity of a microorganism or cell to an antimicrobial or cytotoxic compound, comprises: screening a library of compounds to identify a compound that inhibits an activity of a polypeptide associated with double-stranded DNA break repair or stalled replication fork repair, and producing the identified compound. In other embodiments, the process further comprises derivatizing the identified compound and testing the derivatized compound for its ability to inhibit an activity of a polypeptide associated with DNA repair or replication.

In another related embodiment, a process of producing a compound microbicidal for a drug-resistant microorganism, comprises screening a library of compounds to identify a compound that inhibits an activity of a polypeptide associated with DNA repair or replication and producing the derivatized compound. In further embodiments, the process also comprise derivatizing the identified compound and testing the derivatized compound for its ability to inhibit an activity of a polypeptide associated with DNA repair or replication.

Other embodiments of the invention provide processes of producing a compound cytotoxic for a drug-resistant tumor cell, comprising screening a library of compounds to identify a compound that inhibits an activity of a polypeptide associated with recombination-dependent DNA repair and producing the identified compound. Again, this process may further comprise derivatizing the identified compound and testing the derivatized compound for its ability to inhibit an activity of a polypeptide associated with recombination-dependent DNA repair.

C. Methods of Identifying Inhibitors

The discovery that DNA repair or replication pathways are required to develop resistance of drug-resistant microorganisms and cells provides the basis for methods of identifying inhibitors of DNA repair or replication that are useful in reducing survival of drug-resistant microorganisms and cells and/or increasing the sensitivity of microorganisms and cells to antimicrobial and chemotherapeutic agents. These methods can be used to test one or more candidate inhibitors or screen a library of compounds.

In certain embodiments, methods of identifying compounds and compositions that inhibit homologous recombination are based upon the identification of an inhibitor that binds to or inhibits an activity of a polypeptide involved in DNA repair or replication, including any polypeptide described herein. In particular embodiments, the polypeptide is RecB, RecA, or PriA. Methods of identifying molecules that bind to a polypeptide are widely available and known in the art. The skilled artisan would be fully apprised as to methods of screening or testing molecules to determine their ability to specifically bind a particular polypeptide, based upon general knowledge available in the art, in light of the particular type of molecule being screened.

In one embodiment, the invention provides a general method of identifying an agent that increases the microbicidal activity of an antimicrobial compound (or the antineoplastic activity of a chemotherapeutic agent), comprising: (a) screening one or more candidate agents for their ability to bind a polypeptide associated with RNA repair or replication; and (b) identifying one or more agents that bind to said polypeptide.

In another embodiment, the invention provides a general method of identifying an agent that is microbicidal or cytotoxic for a drug-resistant microorganism or cell, comprising: (a) screening one or more candidate agents for their ability to bind a polypeptide associated with DNA repair or replication; and (b) identifying one or more agents that bind to said polypeptide.

In one embodiment, inhibitors are identified by screening libraries of molecules or chemical compounds, e.g., small molecules. Such libraries and methods of screening the same are known in the art and include: biological libraries, natural products libraries, spatially addressable parallel solid phase or solution phase libraries, synthetic library methods requiring deconvolution, the ‘one-bead one-compound’ library method, and synthetic library methods using affinity chromatography selection. The biological library approach is largely limited to polypeptide libraries, while the other four approaches are applicable to polypeptide, non-peptide oligomer or small molecule libraries of compounds. See Lam, K. S. (1997) Anticancer Drug Des. 12:145. In certain embodiments, screening of libraries is performed using an array or microarray, which permits the testing of multiple compounds, e.g., small molecules, polypeptides, or antibodies) simultaneously. In particular embodiments, screening is high throuput screening.

In one embodiment, inhibitors are identified using Automated Ligand Identification System (referred to herein as “ALIS”). See, e.g., U.S. Pat. Nos. 6,721,665, 6,714,875, 6,694,267, 6,691,046, 6,581,013, 6,207,861, and 6,147,344. ALIS is a high-throughput technique for the identification of small molecules that bind to proteins of interest (e.g., RecB, PriA, or RecA). Small molecules found to bind tightly to a protein can then be tested for their ability to inhibit the biochemical activity of that protein.

Thus, in some embodiments, a target protein (e.g., RecB, RecA, or PriA) is mixed with pools of small molecules. Preferably, more than 1,000 pools are used, more preferably more than 2,000 pools are used, more preferably more than 3,000 pools are used, or more preferably, more than 10,000 pools are used. Each pool contains approximately, 1,000 compounds, more preferably approximately 2,500 compounds, or more preferably approximately 5,000 compounds that are ‘mass encoded,’ meaning that their precise molecular structure can be determined using only their mass and knowledge of the chemical library.

The small molecules and proteins are mixed together and allowed to come to equilibrium (they are incubated together for 30 minutes at room temperature). The mixture is rapidly cooled to trap bound complexes and subject to rapid size exclusion chromatography (SEC). Small molecules that bind tightly to the protein of interest will be co-excluded with the protein during SEC. Mass spectroscopic analysis is performed to determine the masses of all small molecules found to bind the protein. Measurement of these masses allows for the rapid determination of the molecular structures of the small molecules.

In certain embodiments, such screening methods further comprise testing agents identified based upon their ability to bind a component of a DNA repair or replication pathway for their ability to increase the microbicidal activity of an antimicrobial compound or increase the cytotoxic activity of a chemotherapeutic compound. In other embodiments, such screening methods further comprise testing agents identified based upon their ability to bind a component of a DNA repair or replication pathway for microbicidal or cytotoxic activity against drug-resistant microorganisms or cells.

In a further embodiment, a peptide or polypeptide that binds a polypeptide component of a DNA repair or replication pathway is identified using phage display methods.

In other related embodiments, inhibitors of DNA repair or replication are identified based upon their ability to interfere with one or more enzymatic or biological activities of a polypeptide associated with DNA repair or replication. In various embodiments, such polypeptides include one or more of RecBC(D)'s helicase, ATPase, or nuclease activities, or PriA or RuvAB's helicase activity. A variety of in vitro and in vivo assays are known and available for measuring helicase, ATPase, and nuclease activities and any may be used according to the invention. In certain embodiments, such assays are performed using recombinantly-produced polypeptides involved in DNA repair or replication, e.g., RecBC(D)-mediated homologous recombination. Such polypeptides may be used individually, e.g., RecB, RecA, or PriA, or in combination, e.g., RecBC(D).

In other embodiments, functional assays to identify inhibitors of DNA repair or replication include whole cell assays. For example, in one embodiment, whole cell screens are performed to identify inhibitors of DNA repair or replication that sensitive cells to an antimicrobial or chemotherapeutic agent, such as drugs that target topoisomerases, e.g., topoisomerase poisons. In addition, in certain embodiments, methods of identifying inhibitors of DNA repair or replication comprise screening potential inhibitors, or libraries thereof, to identify inhibitors that sensitize both wild-type E. coli and E. coli comprising one or both of S83L and parC mutations to an antibiotic.

Whole cell assays of the present invention are not limited to those designed to identify an inhibitor that targets a particular pathway or polypeptide associated with DNA repair or replication. Rather, in certain embodiments, whole cell assays of the present invention are used to identify an inhibitor, based directly upon its ability to enhance sensitivity of a microorganism or cell to an antimicrobial or cytotoxic agent. The ability of an identified inhibitor to inhibit an activity or expression of a polypeptide associated with DNA repair or replication may be confirmed in a separate assay.

For example, in particular embodiments, inhibitors that hypersensitize mammalian cells to topoisomerase poisons are identified by standard HTS screening of libraries of small molecules. Targets of these agents are identified by standard chemical genomics methods. Identified targets are subjected to standard SAR and optimization schemas. In particular embodiments, such screens are performed using cells with mutations in their topoisomerases (essentially the equivalent of a synthetic lethal screen on gyrA). In other embodiments, such screes are used to identify inhibitors that hypersensitize cells with mutant topoisomerases to the original topoisomerase poisons.

In one particular embodiment of whole cell screens to identify a compound that enhances the sensitivity of a microorganism or cell to an antimicrobial or cytotoxic agent, the method involves contacting a microorganism or cell with a candidate compound in the presence of an antimicrobial or cytotoxic agent, and then determining whether said microorganism or cell has increased sensitivity to the antimicrobial or cytotoxic agent as compared to a microorganism or cell that is not treated with the candidate compound. Increased sensitivity indicates that the candidate compound enhances the sensitivity of the microorganism or cell to the antimicrobial or cytotoxic agent.

These methods can be conducted using any microorganism or cell, as well as any antimicrobial or cytotoxic agent, including those described herein. In particular embodiments, the method is conducted using a fluoroquinolone, e.g., ciprofloxacin.

In particular embodiments, the method is conducted using a microorganism or cell contains a mutation in a gene encoding a polypeptide associated with DNA repair or replication, such as, e.g., a mutation in S83 or D87 of gyrA or S80 of parC.

In another particular embodiment of whole cell screens, the invention includes a method of identifying a compound that inhibits induction of the SOS response pathway, mutagenesis, and/or drug resistance induced by an antimicrobial or cytotoxic agent, wherein said method includes contacting a microorganism or cell with a candidate compound in the presence of a sublethal dose of an antimicrobial or cytotoxic agent, wherein said micoorganism comprises an SOS pathway-inducible reporter gene, and determining whether expression of the reporter polypeptide is reduced in the microorganism or cell contacted with the candidate compound as compared to a microorganism or cell comprising said polynucleotide that is not treated with the candidate compound. Reduced expression of the reporter gene indicates that the compound enhances the sensitivity of the microorganism of cell to the antimicrobial or cytotoxic agent.

A reporter gene construct generally comprises a polynucleotide containing an inducible promoter and encoding a reporter polypeptide. A variety of reporter polypeptides are known and available in the art, including, e.g., luciferase. In one embodiment of this aspect of the present invention, the SOS pathway inducible promoter sequence includes a portion of a promoter or enhancer sequence of a gene known to be induced in response to SOS pathway activation, such as, e.g., an error-prone polymerase gene.

Various embodiments of the function-based and whole cell assays described here include the step of determining whether an identified inhbitor reduces or inhibits a DNA repair, recombination, or replication pathway in a microorganism or cell from the one used in the initial assay. For example, in one embodiment, an initial assay is performed to identify inhibitors of bacterial DNA repair, recombination, or replication. Identified inhibitors are then assayed for their ability to inhibit a related pathway in a mammalian cell. Such methods may be employed to identify microorganism or cell-specific inhibitors, which are particularly useful for treating an infection in a mammalian patient.

Whole cell screening assays may be performed using a library of candidate compounds and can be performed using high throuput methods, such as the utilization of microtitre plates comprising multiple wells that can be assayed simultaneously, e.g., using a fluorescence plate reader device.

In certain embodiments, inhibitors of RecBC(D) are identified based upon their ability to interfere with or reduce RecBC(D) helicase or hydrolysis activities. In particular embodiments, RecBC(D) exonuclease or endonuclease activity is examined. The dual enzymatic activities (i.e., ATP hydrolysis and DNA unwinding) of RecBC(D) provide two different assays in which to characterize its activity in vitro. ATP hydrolyzing enzymes generate Pᵢ, ADP, and H⁺. Techniques have been developed to monitor the formation of each of these species. For example, one technique utilizes the enzyme pyruvate kinase to convert ADP back into ATP, generating pyruvate from phosphoenolpyruvate in the process. A second enzyme, L-lactate dehydrogenase uses NADH to convert pyruvate to lactate, and the resulting decrease in absorbance at 340 nm is readily monitored with a standard plate reader (Kiinitsa, K. et al., Anal. Biochem. 321:266-271 (2003).

A more direct means of observing helicase activity is to utilize a DNA substrate that is labeled on complementary strands with a fluorophore-quencher pair. Unwinding of the DNA by RecBC(D) is accompanied by a marked increase in fluorescence as the distance between the two probes increases (Lucius, A. L., et al., J. Mol. Biol. 339:731-750 (2064).

E. coli strains bearing the temperature sensitive mutation parE10(Ts) are dependent on PriA for viability at the non-permissive temperature where topoisomerase IV is inactive (Michel et al, J. Bact. 186:1197-1199, 2004). Thus, inhibitors of PriA (or other steps in the repair pathway) may be identified by screening for molecules that kill this strain at the non-permissive temperature.

Salmonella typhimurium strains bearing the temperature sensitive gyrA208 or gyrB652 mutations are dependent on RecBC(D) function for viability (Bossi et al., Mol. Microb. 21:111-122, 1996). These are believed to mimic the phenotype of gyrA FQ resistance mutations. Accordingly, in certain embodiments, inhibitors are identified by screening at the non-permissive temperature for small molecules that are lethal to this strain.

Inhibitors may be identified by screening E. coli bearing the gyrAS83L or other mutations in the FQ binding site of GyrA for molecules that inhibit cell growth or sensitize the cells to antibiotic treatment, e.g., treatment with fluoroquinolone.

In other embodiments, inhibitors of DNA repair or replication, e.g., RecBC(D)-mediated homologous recombination (and other homologous and non-homologous recombination pathways), are identified by structural analysis, using molecular modeling software tools, which create realistic 3-D models of molecules structures. Such methods include the use of, e.g., molecular graphics (i.e., 3D representations) and computational chemistry (e.g., calculations of the physical and chemical properties).

Using molecular modeling, rational drug design programs can predict which of a collection of different drug like compounds may fit into the active site of an enzyme, and by computationally adjusting their bound conformation, decide which compounds actually might fit the active site well. See William Bains, Biotechnology from A to Z, 2nd edition, Oxford University Press, 1998, at 259.

Basic information on molecular modeling is known and available in the art: e.g., M. Schlecht, Molecular Modeling on the PC, 1998; John Wiley & Sons; Gans et al., Fundamental Principals of Molecular Modeling, 1996, Plenum Pub. Corp.; N. C. Cohen (editor), Guidebook on Molecular Modeling in Drug Design, 1996, Academic Press; and W. B. Smith, Introduction to Theoretical Organic Chemistry and Molecular Modeling, 1996. U.S. patents that provide detailed information on molecular modeling include U.S. Pat. Nos. 6,093,573; 6,080,576; 5,612,894; 5,583,973; 5,030,103; 4,906,122; and 4,812,12.

For example, in one embodiments, the 3-dimensional structure of RecBC(D) (Singelton, M. R. et al., Nature 432:187-93 (2004)) is used according to methods well known in the art to enable the selection of candidate binders from a virtual library of compounds using methods of molecular modeling and docking. In particular embodiments, candidate binders are selected to bind a particular region of RecBC(D), such as, e.g., a chi cutting site, a region that forms “tunnels,” or a region required for nuclease activity, e.g., exonuclease or endonuclease.

The present invention permits the use of molecular and computer modeling techniques to design, and select compounds (e.g., inhibitors) that bind to a polypeptide associated with DNA repair or replication and for which a molecular structure has been determined or can be predicted.

This invention also enables the design of compounds that act as non-competitive inhibitors of DNA repair or replication. These inhibitors may bind to, all or a portion of, an active site of, e.g., RecA or RecB. Similarly, non-competitive inhibitors that bind to either RecA or RecB and inhibit RecA or RecB (whether or not bound to another chemical entity) may be designed using the atomic coordinates of RecA or RecB.

As noted, the crystal structure of RecBC(D) bound to a DNA substrate has been determined (Singleton, M. R. et al., Nature 432:187-93 (2004). In addition, the crystal structure of RecA polypeptides has also been determined (Xing, X. and Bell, C. E. J., J. Mol Biol. 342:1471-85 (2004)). These structures provide insight regarding important functional domains that might be targeted to interfere with their function, and provide the basis for molecular modeling of inhibitors. Thus, structural features of inhibitors can be determined from the relevant crystal structure information.

In further embodiments, the present invention enables computational screening of small molecule databases for chemical entities, agents, or compounds that can bind in whole, or in part, to a polypeptide involved in DNA repair or replication, e.g., RecB, PriA, or RecA, and, thereby prevent homologous recombination, non-homologous recombination, or repair of stalled replication forks. In this screening technique, the quality of fit of such entities or compounds to the binding site may be judged either by shape complementarity or by estimated interaction energy. See Meng, E. C. et al., J. Coma. Chem., 13: 505-524 (1992).

The design of compounds that bind to or inhibit one or more activities of a polypeptide involved in DNA repair or replication, e.g., RecB, PriA, or RecA, according to this invention generally involves consideration of two factors. First, the compound must be capable of physically associating with the target polypeptide. Non-covalent molecular interactions important in the association of compounds with target polypeptides include hydrogen bonding, van der Waals and hydrophobic interactions. Second, the compound must be able to assume a conformation that allows it to associate with a target polypeptide. Although certain portions of the compound will not directly participate in this association with a target polypeptide, those portions may still influence the overall conformation of the molecule. This, in turn, may have a significant impact on potency. Such conformational requirements include the overall three-dimensional structure and orientation of the chemical entity or compound in relation to all or a portion of the active site of a target polypeptide or the spacing between functional groups of a compound comprising several chemical entities that directly interact with a target polypeptide.

The potential inhibitory or binding effect of a chemical compound on DNA repair or replication may be analyzed prior to its actual synthesis and by the use of computer modeling techniques. If the theoretical structure of the given compound precludes any potential association between it and a target polypeptide, synthesis and testing of the compound is obviated. However, if computer modeling suggests a strong interaction is possible, the molecule may then be synthesized and tested for its ability to bind a target polypeptide and inhibit an activity associated with DNA repair or replication, such as, e.g., homologous recombination or fork repair. In this manner, synthesis of inactive compounds may be avoided.

One skilled in the art may use one of several methods to screen chemical entities fragments, compounds, or agents for their ability to associate with a target polypeptide. This process may begin by visual inspection of, for example, the active site of a target polypeptide identified based upon actual or predicted structural information. Selected chemical entities, compounds, or agents may then be positioned in a variety of orientations, or docked, within an individual binding pocket of a target polypeptide. Docking may be accomplished using software such as Quanta and Sybyl, followed by energy minimization and molecular dynamics with standard molecular mechanics force fields, such as CHARMM or AMBER.

Specialized computer programs also assist in the process of selecting chemical entities. These include but are not limited to GRID (Goodford, P. J., J. Med. Chem. 28:849-857 (1985)). GRID is available from Oxford University, Oxford, UK; MCSS (Miranker, A. et al., Structure, Function and Genetics, (1991) Vol. 11, 29-34), MCSS is available from Molecular Simulations, Burlington, Mass., AUTODOCK (Goodsell, D. S. and A. J. Olsen, “Automated Docking of Substrates to Proteins by Simulated Annealing” Proteins: Structure, Function, and Genetics, 8, 195-202 (1990)). AUTODOCK is available from Scripps Research Institute, La Jolla, Calif.; DOCK (Kuntz, I. D. et al., J. Mol. Biol., 161:269-288 (1982)). DOCK is available from University of California, San Francisco, Calif.

Once suitable chemical entities, compounds, or agents are selected, they can be assembled into a single compound or inhibitor. Assembly may proceed by visual inspection of the relationship of the fragments to each other on the three-dimensional image displayed on a computer screen in relation to the atomic coordinates of a target polypeptide. This is followed by manual model building using software such as Quanta or Sybyl.

Useful programs to aid one of skill in the art in connecting the individual chemical entities, compounds, or agents include but are not limited to CAVEAT (Bartlett, P. A. et al, “CAVEAT: A Program to Facilitate the Structure-Derived Design of Biologically Active Molecules”. In Molecular Recognition in Chemical and Biological Problems”, Special Pub., Royal Chem. Soc., 78:82-196 (1989)). CAVEAT is available from the University of California, Berkeley, Calif.; 3D Database systems such as MACCS-3D (MDL Information Systems, San Leandro, Calif.). This area is reviewed in Martin, Y. C., J. Med. Chem. 35:2145-2154 (1992); also HOOK (available from Molecular Simulations, Burlington, Mass.).

Instead of designing an inhibitor in a step-wise fashion, one chemical moiety at a time, as described above, inhibitors may be designed as a whole or “de novo” using either an empty binding site or optionally including some portion(s) of known inhibitor(s). These methods include LUDI (Bohm, H.-J., J. ComR. Aid. Molec. Design 6:61-78 (1992)). LUDI is available from Biosym Technologies, San Diego, Calif. and LEGEND (Nishibata, Y. and A. Itai, Tetrahedron 47:8985 (1991)). LEGEND is available from Molecular Simulations, Burlington, Mass. LeapFrog is available from Tripos Associates, St. Louis, Mo.

Other molecular modeling techniques can also be employed in accordance with this invention. See, e.g., Cohen, N. C. et al., J. Med. Chem. 33:883-894 (1990). See also, Navia, M. A. and M. A. Murcko, Current Opinions in Structural Biology 2:202-210 (1992).

Once a compound has been designed or selected by the above methods, the efficiency with which that compound may bind to a component of a DNA repair or replication pathway and inhibit its activity may be tested and optimized by computational evaluation. An effective inhibitor of DNA repair or replication preferably demonstrate a relatively small difference in energy between its bound and free states (i.e., a small deformation energy of binding). Thus, the most efficient inhibitors should preferably be designed with deformation energy of binding of not greater than about 10 kcal/mole, or more preferably, not greater than 7 kcal/mole.

Once an inhibitor has been optimally selected or designed, as described above, substitutions can then be made in some of its atoms or side groups to improve or modify its binding properties. Generally, initial substitutions are conservative, e.g., the replacement group will have approximately the same size, shape, hydrophobicity and charge as the original group. It should, of course, be understood that components known in the art to alter conformation should be avoided, unless such changes are desired for the particular application at issue. Such substituted chemical compounds can then be analyzed for efficiency of fit into the 3-D structures of a target polypeptide by the same computer methods described in detail, above.

D. Methods of Use

The present invention establishes that DNA repair or replication pathways are utilized for survival of drug resistant microorganisms and cells. In addition, the present invention establishes that inhibition of DNA repair or replication causes microorganisms and cells to be more sensitive to antimicrobial and chemotherapeutic agents. Accordingly, the invention includes the use of inhibitors of DNA repair or replication for a variety of purposes related to killing drug-resistant microorganisms and cells or increasing the sensitivity of microorganisms and cells to antimicrobial and chemotherapeutic agents, including, but not limited to, any disclosed herein.

In one embodiment, the present invention includes a method of sensitizing a microorganism or cell to an antimicrobial or chemotherapeutic agent. The method involves contacting a microorganism or cell with an inhibitor of DNA repair or replication. In a related embodiment, a method of increasing the microbicidal or cytotoxic activity of an antimicrobial or cytotoxic agent includes contacting a microorganism or cell with an inhibitor of DNA repair or replication in combination with an antimicrobial or cytotoxic agent.

As described throughout, in certain applications of the present invention, inhibitors are administered to a subject or contacted with a microorganism or cell in combination with an antimicrobial or cytotoxic agent. This may occur at the same time, or the inhibitor may be administered or contacted before or after administration or contact with the agent.

Since the methods of the present invention may be used to sensitize a micoorganism or cell to a drug, in related embodiments, the present invention includes methods of reducing the minimum inhibitory concentration (MIC) of a drug and methods of shifting the therapeutic index of a drug, such that a lower dosage may be used, when the drug is provided in combination with an inhibitor of DNA repair or replication.

The ratio of the drug dose that produces an undesired effect to the dose that causes the desired effects is a therapeutic index and indicates the selectivity of the drug and consequently its usability. It should be noted that a single drug can have many therapeutic indices, one for each of its undesirable effects relative to a desired drug action, and one for each of its desired effects if the drug has more than one action. Accordingly, by using an inhibitor of the present invention in combination with a drug, thereby enhancing the sensitivity of a microorganism or cell to the drug and, thus, decreasing the drug dose required for a desired effect, the present invention provides a method of increasing the therapeutic index.

Generally, an increase in the microbicidal or cytotoxic activity of an agent, i.e., drug, is determined using methods routinely available in the art, including, e.g., determining the MIC of the agent in the presence or absence of the inhibitor of DNA repair or replication. In various embodiments, an inhibitor increases the microbicidal or cytotoxic activity of an agent by at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 60-fold, 70-fold, 80-fold, 90-fold or 100-fold. In related embodiments, an inhibitor reduces the MIC of an agent by at least 5%, 10%, 15%, 20%, 25%, 30%, 35%, 40%, 45%, 50%, 55%, 60%, 65%, 70%, 75%, 80%, 85%, 90%, or 95%. In other embodiments, an inhibitor shift the therapeutic index of an agent, such that a patient may be treated with a dosage that is at least 5%, 10%, 15%, 20%, 25%, 30%, 35%, 40%, 45%, 50%, 55%, 60%, 65%, 70%, 75%, 80%, 85%, 90%, or 95% lower than the dosage used in the absence of an inhibitor.

Accordingly, the invention further provides methods of treating a subject diagnosed with or suspected of having an infection with a microorganism, comprising providing to the subject an appropriate antimicrobial agent in combination with an inhibitor of the present invention. In particular embodiments, the antimicrobial agent is provided at a dosage lower than previously used (i.e., in the absence of an inhibitor of the present invention).

Similarly, in other related embodiments, the invention further provides methods of treating a subject diagnosed with or suspected of having a tumor, comprising providing to the subject an appropriate chemotherapeutic agent in combination with an inhibitor of the present invention. In particular embodiments, the chemotherapeutic agent is provided at a dosage lower than previously used (i.e., in the absence of an inhibitor of the present invention).

The invention further includes a method of treating a subject diagnosed with or at risk of having a microbial infection, comprising providing an inhibitor of DNA repair or replication to said patient. In a related embodiment, the inhibitor is provided in combination with an antimicrobial agent.

In addition, the invention includes a method of treating a subject diagnosed with or suspected of having a tumor, comprising providing an inhibitor of DNA repair or replication to said patient. In a related embodiment, the inhibitor is provided in combination with a chemotherapeutic agent.

The methods described herein related to increasing or enhancing the activity of an antimicrobial or chemotherapeutic agent allow the use of dosages lower than those previously demonstrated effective in the absence of an inhibitor of the present invention. Such lower dosages offer significant advantages, including decreased side effects and decreased costs. Accordingly, in certain embodiments, methods of the present invention are practiced using dosages of antimicrobial or chemotherapeutic agent lower than those previously used. In addition, in particular embodiments, methods of the invention are practiced using antimicrobial or chemotherapeutic agents not generally used due to prohibitive side effects or high cost. For example, sparfloxacin is associated with a high incidence of photosensitivity, grepafloxacin is associated with QTc prolongation, and lomefloxacin is associated with a high incidence of photosensitivity.

The invention also provides methods of combating drug-resistant microorganisms and cells. Such methods may be used to reduce the growth of or kill drug-resistant microorganisms and cells. Depending upon the particular application of the method, the method typically comprises providing an inhibitor of DNA repair or replication to a subject or contacting a drug-resistant microorganism or cell with an inhibitor of DNA repair or replication. In one embodiment, the inhibitor is provided in combination with an antimicrobial or cytotoxic agent. For example, an inhibitor of the present invention can be used in combination with any antibiotic disclosed herein or otherwise known in the art. In certain embodiments, an inhibitor is used in combination with rifampin, an oxazolidinone (e.g., linezolid), a quinolone, a fluoroquinolone (e.g., ciprofloxacin, levofloxacin, moxifloxacin, gatifloxacin, gemifloxacin, ofloxacin, lomefloxacin, norfloxacin, enoxacin, sparfloxacin, temafloxacin, trovafloxacin, grepafloxacin), a macrolide (e.g., azithromycin and clarithromycin), or a later generation cephalosporin (e.g., cefaclor, cefadroxil, cefazolin, cefixime, cefoxitin, cefprozil, ceftazidime, cefuroxime, and cephalexin).

The inhibitors of the present invention can be administered to, provided to, or contacted with microorganisms or cells that are located within or on a subject. For example, the inhibitors may be provided to a subject having a microbial infection or tumor. Alternatively, the inhibitors may be contacted with microorganisms or cells that are not present within or on a subject. For example, inhibitors can be used to treat or kill a microorganism on a solid surface, such as a food preparation surface, or inhibitors can be used to treat or kill, or prevent the growth of, a microorganism in a food or beverage or pharmaceutical or cosmetic preparation.

In various embodiments, the methods of the invention are applied to any of a wide variety of microorganisms and cells, including all those described herein.

In certain embodiments, a method of the invention is applied to bacteria. In particular embodiments, the bacteria are gram positive or gram negative. In further embodiments, the bacteria are sensitive or resistant to one or more antibiotics. In particular embodiments, the bacteria comprise one or more mutations in a gene encoding a type II topoisomerase, e.g., the gyrase or topoisomerase gene, wherein the mutations are associated with drug resistance. The protein targets for certain antibiotics, e.g., quinolones, are type II topoisomerases (DNA gyrase and topoisomerase IV). Both are tetrameric enzymes with two A subunits and two B subunits, encoded by the gyrA and gyrB genes; respectively, in the case of DNA gyrase, and by the parC and parE genes in the case of topoisomerase IV. There is a region in these genes that is known as the quinolone resistance determining region (QRDR), where mutations associated with the acquisition of quinolone resistance have been located. Specific mutations identified as playing important roles in the acquisition of resistance are located in the QRDR of the gyrA and parC genes. Specific mutations identified as being associated with drug resistance include, e.g., mutation of amino acid resides Ser91 and Asp95 of GyrA and Glu91 and Ser87 of ParC. Furthermore, double mutations in Ser91 and Asp95 of GyrA plus mutation of Glu91 or Ser87 of ParC lead to significant high level drug resistance.

Accordingly, in particular embodiments, methods of the invention are applied to the treatment of bacteria having one or more mutations in gyrA, e.g., at Ser91 and/or Asp95, or having one ore more mutations in parC, e.g., Glu91 and/or Ser87. In a particular embodiment, a bacteria has one or more mutations in GyrA, as well as one or more mutations in ParC, including, but not limited to, the specific mutations described herein.

Relatedly, the invention includes methods of diagnosing the presence of a drug-resistant microorganism, e.g., bacteria, determining whether a microorganism has acquired drug resistance, and determining appropriate therapeutic treatment of a microorganism (or a patient infected with a microorganism), comprising determining the presence of a mutation associated with drug resistance in a microorganism. The presence of a mutation can be readily determined by a variety of different methods known and routinely used in the art, including, e.g., PCR analysis. A rapid PCR mismatch method of detecting mutations in gyrA and parC is described, e.g., in Ziang, Y. Z. et al., Journal of Antimicrobial Chemotherapy, 49: 549-552 (2002). In particular embodiments, the invention provides a methods of treating a drug-resistant microorganism, comprising determining the presence of one or more mutations associated with resistance and, if such a mutation is present, providing an inhibitor of DNA repair or replication to the microorganism. The inhibitor may be provided in the presence or absence of another antimicrobial agent.

Examples of bacteria treated according to methods of the invention include, but are not limited to: Baciccis Antracis; Enterococcus faecalis; Corynebacterium; diphtheriae; Escherichia coli; Streptococcus coelicolor; Streptococcus pyogenes; Streptobacillus moniliformis; Streptococcus agalactiae; Streptococcus pneumoniae; Salmonella typhi; Salmonella paratyphi; Salmonella schottmulleri; Salmonella hirshfeldii; Staphylococcus epidermidis; Staphylococcus aureus; Klebsiella pneumoniae; Legionella pneumophila; Helicobacter pylori; Moraxella catarrhalis, Mycoplasma pneumonia; Mycobacterium tuberculosis; Mycobacterium leprae; Yersinia enterocolitica; Yersinia pestis; Vibrio cholerae; Vibrio parahaemolyticus; Rickettsia prowazekii; Rickettsia rickettsii; Rickettsia akari; Clostridium difficile; Clostridium tetani; Clostridium perfringens; Clostridium novyii; Clostridium septicum; Clostridium botulinum; Legionella pneumophila; Hemophilus influenzae; Hemophilus parainfluenzae; Hemophilus aegyptus; Chlamydia psittaci; Chlamydia trachomatis; Bordetella pertusis; Shigella spp.; Campylobacter jejuni; Proteus spp.; Citrobacter spp.; Enterobacter spp.; Pseudomonas aeruginosa; Propionibacterium spp.; Bacillus anthracis; Pseudomonas syringae; Spirrilum minus; Neisseria meningitidis; Listeria monocytogenes; Neisseria gonorrheae; Treponema pallidum; Francisella tularensis; Brucella spp.; Borrelia recurrentis; Borrelia hermsii; Borrelia turicatae; Borrelia burgdorferi; Mycobacterium avium; Mycobacterium smegmatis; Methicillin-resistant Staphyloccus aureus; Vancomycin-resistant enterococcus; and multi-drug resistant bacteria (e.g., bacteria that are resistant to more than 1, more than 2, more than 3, or more than 4 different drugs).

In some embodiments, an inhibitor of the present invention is used to treat an already drug resistant bacterial strain such as Methicillin-resistant Staphylococcus aureus (MRSA) or Vancomycin-resistant enterococcus (VRE), including, but not limited to, any other drug-resistant strain described herein.

Accordingly, the inhibitors herein may be used to treat a wide variety of bacterial infections and conditions, such as intra-abdominal infections, ear infections, gastrointestinal infections, bone, joint, and soft tissue infections, sinus infections, bacterial infections of the skin, bacterial infections of the lungs, urinary tract infections, respiratory tract infections, sinusitis, sexually transmitted diseases, ophthalmic infectionstuberculosis, pneumonia, lyme disease, and Legionnaire's disease. Thus any of the above conditions and other conditions resulting from bacterial infections may be prevented or treated by the compositions herein.

In specific embodiments, methods of the present invention are used to treat any classification of urinary tract infection (UTI), and UTIs caused by any microorganism. Examples of these include, but are not limited to: uncomplicated UTI, of which 85% is caused by E. coli and the remainder by S. saprophyticus, Proteus spp., and Klebseiella spp; complicated UTIs, associated with gram-negative organisms, including E. coli, P. aeuroginosa, and E. facecalis; and recurrent UTIs, 80% of which are caused by an organism different from the organism isolated from the preceding infection, and the remaining 20% are relapses, possibly due to persistence fo infection with the same organisms after therapy. E. coli is the most common bacterium isolated from UTIs and accounts for about 80% of community-acquired infections, while Staphylococcus saprophyticus accounts for about 10%. In hospitalized patients, E. coli accounts for about 50% of cases, the gram-negative species Klebsiella, Proteus, Enterobacter and Serratia account for about 40%, and the gram-positive bacterial cocci Enterococcus faecalis and Staphylococcus spp (e.g., saprophyticus and aureus) account for most of the remainder.

In specific examples of embodiments of the present invention, an inhibitor is used to treat: a respiratory tract infection with Streptococcus, alone or in combination with levofloxacin; a respiratory or urinary tract infection with P. aeuroginosa, alone or in combination with ciprofloxacin; or a urinary tract infection with E. coli, alone or in combination with ciprofloxacin.

In particular embodiments, an inhibitor of the present invention is used to treat a microorganism used in biowarfare. Biowarfare and bioterrorism have been defined as the intentional or the alleged use of viruses, bacteria, fungi and toxins to produce death or disease in humans, animals or plants. Of these various biowarfare agents, bacteria and viruses appear to pose the most significant threat of widespread harm, primarily due to their relative ease of both production and transmissibility, as well as a lack of medical treatments. Examples of known viruses considered suitable as biowarfare agents include smallpox virus, and the hemorrhagic fever viruses, such as ebola virus, amongst others. Although there are currently a limited number of known viruses considered suitable as biowarfare agents, many more might be made suitable through genetic engineering or other modifications. Discussed in Kostoff, R. N. The Scientist 15:6 (2001). Such novel viral agents present a particular threat, since vaccines and methods of detection and treatment would likely not exist.

Examples of biowarfare bacteria and spores that may be treated according to the present invention include, but are not limited to, Bacillus anthracis, Bacillus cereus, Clostridium botulinum, Yersinia pestis, Yersinia enterocolitica, Francisella tularensis, Brucella species, Clostridium perfringens, Burkholderia mallei, Burkholderia pseudomallei, Staphylococcus species, Tuberculosis species, Escherichia coli, Group A Streptococcus, Group B Streptococcus, Streptococcus pneumoniae, Helicobacter pylori, Francisella tularensis, Salmonella enteritidis, Mycoplasma hominis, Mycoplasma orale, Mycoplasma salivarium, Mycoplasma fermentans, Mycoplasma pneumoniae, Mycobacterium bovis, Mycobacterium tuberculosis, Mycobacterium avium, Mycobacterium leprae, Rickettsia rickettsii, Rickettsia akari, Rickettsia prowazekii, Rickettsia canada, and Coxiella burnetti.

Examples of yeast and other fungi that may be treated according to the present invention include, but are not limited to, Aspergillus species (e.g. Aspergillus niger), Mucor pusillus, Rhizopus nigricans, Candida species (e.g. Candida albicans, Candida dubliniensis, C. parapsilosis, C. tropicalis, and C. pseudotropicalis), Torulopsis glabrata, Blastomyces dermatitidis, Coccidioides immitis, Histoplasma capsulatum, Cryptococcus neoformans, and Sporothrix schenckii.

Viral infections that may be treated by the methods and compositions of the present invention include those caused by both DNA and RNA viruses. DNA viruses may comprise a double-stranded DNA genome (e.g., smallpox) or a single-stranded DNA genome (e.g., adeno-associated virus). RNA viruses include those with genomes comprising antisense RNA (e.g., Ebola), sense RNA (e.g., poliovirus), or double-stranded RNA (e.g., reovirus), as well as retroviruses (e.g., HIV-1). Examples of DNA viruses and associated diseases that may be treated by the invention include: variola (smallpox); herpesviruses, such as herpes simplex (cold sores), varicella-zoster (chicken pox, shingles), Epstein-Barr virus (mononucleosis, Burkitt's lymphoma), KSHV (Kaposi's sarcoma), and cytomegalovirus (blindness); adenoviruses; and hepatitis B. Examples of RNA viruses include polioviruses, rhinociruses, rubella, yellow fever, West Nile virus, dengue, equine encephalitis, hepatitis A and C, respiratory syncytial virus, parainfluenza virus, and tobacco mosaic virus. RNA viruses have been implicated in a variety of human diseases that may be treated by the invention, including, for example, measles, mumps, rabies, Ebola, and influenza. Viral infections treated by the invention may be localized to specific cells or tissues, or they may be systemic. In addition, these viral infections may be either lytic or latent.

Compositions and methods of the present invention may, therefore, be used to treat diseases, including, but not limited to, cutaneous anthrax, inhalation anthrax, gastrointestinal anthrax, nosocomical Group A streptococcal infections, Group B streptococcal disease, meningococcal disease, blastomycocis, streptococcus pneumonia, botulism, Brainerd Diarrhea, brucellosis, pneumonic plague, candidiasis (including oropharyngeal, invasive, and genital), drug-resistant Streptococcus pneumoniae disease, E. coli infections, Glanders, Hansen's disease (Leprosy), cholera, tularemia, histoplasmosis, legionellosis, leptospirosis, listeriosis, meliodosis, mycobacterium avium complex, mycoplasma pneumonia, tuberculosis, peptic ulcer disease, nocardiosis, chlamydia pneumonia, psittacosis, salmonellosis, shigellosis, sporotrichosis, strep throat, toxic shock syndrome, trachoma, traveler's diarrhea, typhoid fever, ulcer disease, and waterborne disease.

The methods and compositions of the present invention may also be used to treat systemic viral infections that can lead to severe hemorrhagic fever. Although many viral infections can be associated with hemorrhagic complications, infection with any of several RNA viruses regularly results in vascular involvement and viral hemorrhagic fever. Known viral hemorrhagic fevers include Ebola hemorrhagic fever, Marburg disease, Lassa fever, Argentine haemorrhagic fever, and Bolivian hemorrhagic fever. Etiologic agents for these disease include Ebola virus, Marburg virus, Lassa virus, Junin virsus, and Machupo virus, respectively.

A variety of viruses are associated with viral hemorrhagic fever, including filoviruses (e.g., Ebola, Marburg, and Reston), arenaviruses (e.g. Lassa, Junin, and Machupo), and bunyaviruses. In addition, phleboviruses, including, for example, Rift Valley fever virus, have been identified as etiologic agents of viral hemorrhagic fever. Etiological agents of hemorrhagic fever and associated inflammation may also include paramyxoviruses, particularly respiratory syncytial virus, since paramyxoviruses are evolutionarily closely related to filoviruses (Feldmann, H. et al. Arch Virol Suppl 7:81-100 (1993)). In addition, other viruses causing hemorrhagic fevers in man have been characterized as belonging to the following virus groups: togavirus (Chikungunya), flavivirus (dengue, yellow fever, Kyasanur Forest disease, Omsk hemorrhagic fever), nairovirus (Crimian-Congo hemorrhagic fever) and hantavirus (hemorrhagic fever with renal syndrome, nephropathic epidemia). Furthermore, Sin Nombre virus was identified as the etiologic agent of the 1993 outbreak of hantavirus pulmonary syndrome in the American Southwest.

In other embodiments, an inhibitor of the present invention is used in combination with an antiviral agent, including but not limited: AZT; Ganciclovir; valacyclovir hydrochloride (Valtrex™); Beta Interferon; Cidofovir; Ampligen™; penciclovir (Denavir™), foscarnet (Foscavir™), famciclovir (Famvir™), acyclovir (Zovirax™), and any others recited herein.

Examples of viruses that may be treated according to methods of the present invention include, but are not limited to, human immunodeficiency virus (HIV); influenza; avian influenza; ebola; chickenpox; polio; smallpox; rabies; respiratory syncytial virus (RSV); herpes simplex virus (HSV); common cold virus; severe acute respiratory syndrome (SARS); Lassa fever (Arenaviridae family), Ebola hemorrhagic fever (Filoviridae family), hantavirus pulmonary syndrome (Bunyaviridae family), and pandemic influenza (Orthomyxoviridae family).

In another example, an inhibitor is used in combination with an antiprotozoan agent selected from the group consisting of: Chloroquine; Pyrimethamine; Mefloquine Hydroxychloroquine; Metronidazole; Atovaquone; Imidocarb; Malarone™; Febendazole; Metronidazole; Ivomec™; Iodoquinol; Diloxanide Furoate; and Ronidazole.

Examples of protozoan organisms that are treated using methods of the present invention include, but are not limited to, Acanthameba; Actinophrys; Amoeba; Anisonema; Anthophysa; Ascaris lumbricoides; Bicosoeca; Blastocystis hominis; Codonella; Coleps; Cothurina; Cryptosporidia Difflugia; Entamoeba histolytica (a cause of amebiasis and amebic dysentery); Entosiphon; Epalxis; Epistylis; Euglypha; Flukes; Giardia lambia; Hookworm Leishmania spp.; Mayorella; Monosiga; Naegleria Hartmannella; Paragonimus westermani; Paruroleptus; Plasmodium spp. (a cause of Malaria) (e.g., Plasmodium falciparum; Plasmodium malariae; Plasmodium vivax and Plasmodium ovale); Pneumocystis carinii (a common cause of pneumonia in immunodeficient persons); microfilariae; Podophrya; Raphidiophrys; Rhynchomonas; Salpingoeca; Schistosoma japonicum; Schistosoma haematobium; Schistosoma mansoni; Stentor; Strongyloides; Stylonychia; Tapeworms; Trichomonas spp. (e.g., Trichuris trichiuris and Trichomonas vaginalis (a cause of vaginal infection)); Typanosoma spp.; and Vorticella.

In other embodiments, an inhibitor of the present invention is used in combination with an antifungal agent selected from the group consisting of: imidazoles (e.g., clotrimazole, miconazole; econazole, ketonazole, oxiconazole, sulconazole), ciclopiroz, butenafine, and allylamines.

Examples of fungus infections that can be treated with an inhibitor (+/− an antifungal agent) according to methods of the invention include, but are not limited to, tinea; athlete's foot; jock itch; and candida.

In particular embodiments, the present invention contemplates the prevention and treatment of infectious diseases identified in Table 1, which have re-emerged with increased resistance to medications:

TABLE 1
Examples of Infectious Diseases With Increased Resistance to Medications

| | |
|---|---|
| Cryptosporidiosis | Cryptosporidium parvum |
| | (protozoan) |
| Diphtheria | Corynebacterium diptheriae |
| | (bacterium) |
| Malaria | Plasmodium species |
| | (protozoan) |
| Meningitis, necrotizing fasciitis (flesh- | Group A Streptococcus |
| eating disease), toxic-shock | (bacterium) |
| syndrome, and other diseases | |
| Pertussis (whooping cough) | Bordetella pertussis (bacterium) |
| Rabies | Rhabdovirus group (virus) |
| Rubeola (measles) | Morbillivirus genus (virus) |
| Schistosomiasis | Schistosoma species (helminth) |
| Tuberculosis | Mycobacterium tuberculosis |
| | (bacterium) |
| Yellow fever | Flavivirus group (virus) |
| HIV-associated infections | Staphylococcus (bacteria) |

As discussed earlier, pathways comparable to the bacterial pathways discussed herein are also known to exist in eukaryotic cells. Accordingly, in certain embodiments, inhibitors of the present invention are used to treat eukaryotic cells, including, e.g., mammalian cells. In one embodiment, an inhibitor is used to treat a drug-resistant tumor. In another embodiment, an inhibitor is used in combination with a chemotherapeutic agent to treat a drug-sensitive or drug-resistant tumor. The inhibitors may be used to treat or prevent both benign and malignant tumors.

Examples of cancers that may be treatable or preventable by the compositions and methods of the present invention include, but are not limited to, breast cancer; skin cancer; bone cancer; prostate cancer; liver cancer; lung cancer; brain cancer; cancer of the larynx; gallbladder; pancreas; rectum; parathyroid; thyroid; adrenal; neural tissue; head and neck; colon; stomach; bronchi; kidneys; basal cell carcinoma; squamous cell carcinoma of both ulcerating and papillary type; metastatic skin carcinoma; osteo sarcoma; Ewing's sarcoma; veticulum cell sarcoma; myeloma; giant cell tumor; small-cell lung tumor; gallstones; islet cell tumor; primary brain tumor; acute and chronic lymphocytic and granulocytic tumors; hairy-cell leukemia; adenoma; hyperplasia; medullary carcinoma; pheochromocytoma; mucosal neuromas; intestinal ganglioneuromas; hyperplastic corneal nerve tumor; marfanoid habitus tumor; Wilm's tumor; seminoma; ovarian tumor; leiomyomater tumor; cervical dysplasia and in situ carcinoma; neuroblastoma; retinoblastoma; soft tissue sarcoma; malignant carcinoid; topical skin lesion; mycosis fungoide; rhabdomyosarcoma; Kaposi's sarcoma; osteogenic and other sarcoma; malignant hypercalcemia; renal cell tumor; polycythermia vera; adenocarcinoma; glioblastoma multiforme; leukemias (including acute myelogenous leukemia); lymphomas; malignant melanomas; epidermoid carcinomas; chronic myleoid lymphoma; gastrointestinal stromal tumors; and melanoma.

As indicated in the specific illustrative embodiments recited above, inhibitors of DNA replication and repair, may be used in combination with an antimicrobial or chemotherapeutic agent that targets a DNA replication or repair pathway, such as a fluoroquinolone. However, it is further understood according to the present invention that inhibitors of DNA repair or replication may also be used to enhance sensitivity to agents that act via different mechanisms. Since the inhibitors of DNA repair and replication target fundamental cellular processes, they are generally somewhat crippling to microorganisms and cells, and therefore, synergize or cooperate additively with agents that target other pathways. For example, an inhibitor of RecB would block induction of RecA gene expression mediated via the SOS pathway. Accordingly, the methods of the present invention are applicable to agents that act on DNA repair or replication pathways, as well as agents that act on different cellular targets.

### EXAMPLES

### Example 1

### recA, recB, recG, priA, ruvB and ruvC Mutants Exhibit an Increased Sensitivity to Sublethal Doses of Ciprofloxacin

The contribution of different components of DNA recombination and repair pathways in mediating ciprofloxacin resistance was determined by examining the effect of various mutations. The experiments were performed using the E. coli strain MG1655 as the genetic background, since this K-12 strain was used in the E. coli genome sequencing project. Strains listed in Table 2 were constructed using PCR-mediated gene replacement. See Murphy, K C, et al., Gene 2000, 246:321-330. PCR reactions were performed using Platinum pfx DNA polymerase from Invitrogen, with standard cycling parameters. Genomic template DNA was prepared from a fresh bacterial overnight culture using the DNeasy kit (Qiagen).

The kanamycin cassette was PCR amplified from a pUC4K plasmid using primers 5′-GGA AAG CCA CGT TGT GTC TC and 5′-CGA TTT ATT CAA CAA AGC CGC. Gene specific components from each gene were amplified from MG1655 genomic DNA to obtain two PCR products: the ‘N-fragment’ containing 500 base pairs upstream and including the first two to three codons and the ‘C-fragment’ containing the last two to three codons and 500 base pairs downstream. The fragment ends were engineered to contain the reverse complement of the kanamycin cassette sequence at their internal sites by using primers with 20 base pairs of homology and a 20 base pair tail complementary to the kanamycin cassette ends at the 3′-end for the N fragment and at the 5′-end for the C fragment.

TABLE 2
Mutated strains

| Parent | Mutation |
|---|---|
| MG1655 | — |
| ATCC25922 | — |
| MG1655 | DY329 (nadA::RED) |
| MG1655 | lacZΔ::kan |
| MG1655 | polBΔ::kan |
| MG1655 | polBΔ::spc |
| MG1655 | dinBΔ::kan |
| MG1655 | umuDCΔ::kan |
| MG1655 | umuDCΔ::cat |
| MG1655 | polBΔ::Spc, dinBΔ::kan |
| MG1655 | polBΔ::Spc, umuDCΔ::kan |
| MG1655 | dinBΔ::kan umuDCΔ::cat |
| MG1655 | polBΔ::spc dinBΔ::kan, UmuDC::Cat |
| MG1655 | LexA(S119A)::kan |
| MG1655 | recAΔ::kan |
| MG1655 | recBΔ::kan |
| MG1655 | recDΔ::kan |
| MG1655 | recFΔ::kan |
| MG1655 | recGΔ::kan |
| MG1655 | ruvBΔ::kan |
| MG1655 | ruvCΔ::kan |
| MG1655 | sulAΔ::kan |
| MG1655 | priAΔ::kan |
| ATCC25922 | lacZΔ::kan |
| ATCC25922 | LexA(S119A)::kan |
| ATCC25922 | recFΔ::kan |

To create the full, gene-specific disruption cassettes, the products of the N-fragment, C-fragment and kanamycin cassette reactions were combined in a PCR reaction, in equal volume. Conditions for this PCR reaction were standard, with the exception that the proximal primers were used in limiting amounts. The excess distal primer is consumed in the second PCR reaction. The complementary sequences on the N- and C-fragments acted as primers for the kanamycin cassette, which resulted in a final product containing approximately 500 base pairs of upstream sequence, the kanamycin cassette in a reverse orientation to the gene that was knocked out, and 500 base pairs of downstream sequence.

Generation of the genomic deletions in MG1655 proceeded in two steps: (i) genomic insertion into strain MG-DY329 and (ii) P1-mediated transfer of the deletion cassette to MG1655. In the first step, the linear DNA fragments (PCR products) were electroporated into the hyper-recombinational E. coli strain MG-DY329 [Yu, D, et al. Proc Natl Acad Sci USA (2000) 97:5978-5983], a derivative of MG1655 which carries the lambda phage red genes. This strain accepted the linear PCR product and recombined it into the genome with high efficiency. Recombination genes were activated by growing DY329 at 42° C. and the competent cells stored at −80° C. The competent cells were transformed with the desired kanamycin cassette and kan transformants selected at 30° C.

Although MG-DY329 was engineered such that the lambda phage red genes could be easily removed to return the cell to a non-hyper-recombinational background, P1 transduction was utilized to move the gene-specific disruption from MG-DY329 into MG1655. MG1655 provides a more ‘wild-type’ background than MG-DY329, and thus simplifies the interpretation of the results. Gene deletions were verified by PCR.

The ΔlacZ strain was constructed as a control. The ΔlacZ strain exhibited wild-type growth and mutation (+/−1.15-fold) and is, therefore, also referred to herein as “wild-type.”

TABLE 3
Doubling time and sensitivity to ciprofloxacin of E. coli mutants.

| | Relative | ciprofloxacin MIC (ng/ml) | | |
|---|---|---|---|---|
| E. coli strain | Doubling Time | WT gyrA | gyrA S83Δ | gyrA S83L |
| MG1655 | 1.00 (±0.00) | 35 | nd | 500 |
| ΔlacZ | 1.03 (±0.01) | 35 | 250 | 500 |
| ΔdinB | 1.01 (±0.03) | 35 | 250 | 500 |
| ΔumuDC | 1.02 (±0.02) | 35 | 250 | 500 |
| ΔpolB, ΔdinB, | 1.09 (±0.13) | 30 | 250 | 500 |
| ΔumuDC | | | | |
| lexA(S119A) | 1.00 (±0.01) | 30 | 250 | 350 |
| ΔrecD | 1.01 (±0.02) | 30 | nd | 350 |
| ΔrecF | 1.02 (±0.02) | 40 | nd | 400 |
| ΔrecO | 0.99 (±0.01) | 35 | nd | 500 |
| ΔrecR | 0.99 (±0.02) | 35 | nd | 350 |
| ΔuvrB | 0.99 (±0.01) | 30 | nd | 400 |
| ΔrecQ | 0.97 (±0.02) | 30 | nd | 500 |
| ΔrecA | 1.13 (±0.10) | 5 | nd | nd |
| ΔrecB | 1.22 (±0.14) | 5 | nd | nd |
| ΔrecG | 1.01 (±0.03) | 10 | nd | 150 |
| ΔruvB | 1.08 (±0.01) | 10 | nd | 150 |
| ΔruvC | 1.07 (±0.10) | 10 | nd | 150 |

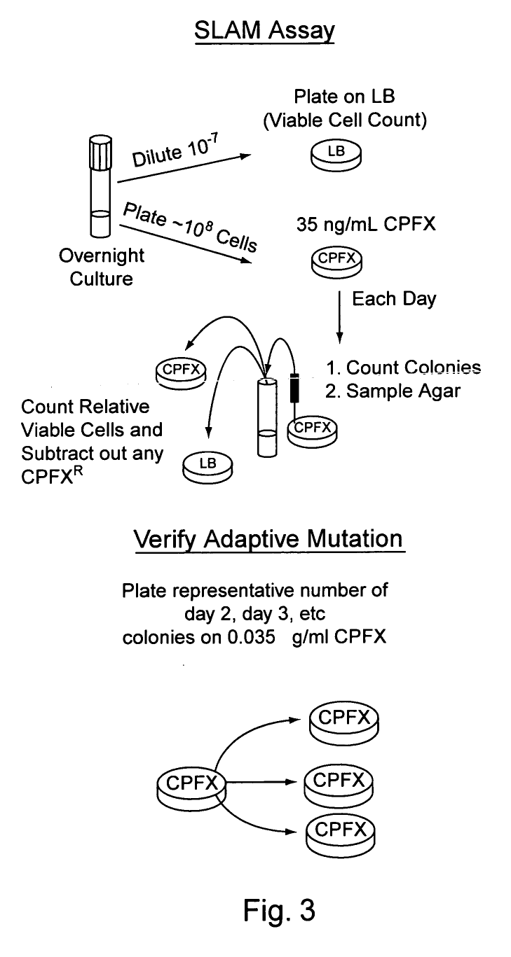

The ciprofloxacin MIC was determined for the wild-type and mutant strains and is provided in Table 3 (WT gyrA column). The MIC for wild-type was 35 ng/ml in liquid media. On solid media, 40 ng/ml ciprofloxacin killed 99% of the cells within 24 hours of plating (FIG. 3). The majority of deletions had little or no effect on the MIC.

Surprisingly, however, the MIC for both the ΔrecA and ΔrecB strains was only 5 ng/ml, indicating that these strains had increased sensitivity to ciprofloxacin as compared to wild-type (although both exhibited virtually wild-type viability in the absence of ciprofloxacin). In addition, no pre- or post-exposure ciprofloxacin resistant mutants were observed in SLAM assays on the ΔrecA or ΔrecB strains (data not shown; see Example 2), indicating that these deletions prevented either the emergence or maintenance of ciprofloxacin resistance.

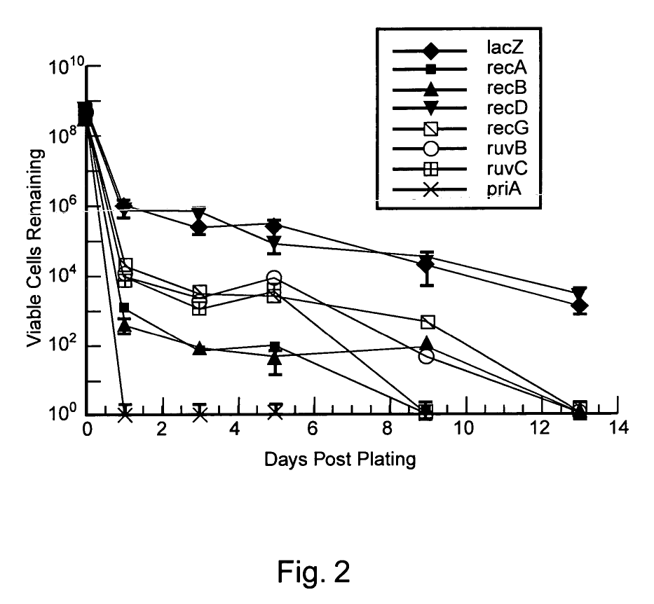

In contrast, deletion of recD had little or no effect on drug sensitivity (Table 3 and FIG. 2), mutation rate, or mutation spectrum. This result is consistent with the fact that RecBC can process DSEs and load RecA onto ssDNA in the absence of the RecD helicase.

The potential steps before and after RecBC(D) and RecA-mediated recombination were examined with ΔrecG, ΔruvB, and ΔruvC strains, since RecG, RuvB, and RuvC are known to be involved in the regression of stalled replication forks and/or the processing of HR intermediates. Deletion of RecG, ruvB, or ruvC did not cause a significant decrease in viability in the absence of ciprofloxacin, but did show high sensitivity to the drug, although not as great as the ΔrecA and ΔrecB strains (Table 3 and FIG. 2). Also, like the ΔrecA and ΔrecB strains, no ciprofloxacin-resistant mutants were isolated from SLAM assays (FIG. 3; see Example 2) on the ΔrecG, ΔruvB, and ΔruvC strains, either before or after exposure to ciprofloxacin. Furthermore, although it was possible to delete recG, ruvB, and ruvC in a gyrA(S83L) background by P1 transduction, the three double mutants were synthetically sick, exhibited increased filamentation relative to the respective single mutants, and had a low ciprofloxacin MIC relative to the gyrA(S83L) parent strain (Table 3). These results demonstrate that the functions of RecG, RuvAB, and RuvC are required in the presence of ciprofloxacin or certain gyrase mutations that confer ciprofloxacin resistance.

To determine if the resumption of processive DNA synthesis is required in response to ciprofloxacin, a ΔpriA strain was examined. Deletion of priA resulted in extreme sensitivity to ciprofloxacin (MIC<1 ng/ml), demonstrating that replication restart is essential in response to the drug (FIG. 2). No mutants were isolated before or after exposure to ciprofloxacin, and a gyrA(S83L) ΔpriA double mutant could not be constructed.

The unexpected results of these studies establish that the RecBC(D)-mediated HR plays an important role in DNA repair processes important for the survival of drug resistant strains having compromised gyrase function, and that inhibition of DNA repair and replication pathways renders bacteria more sensitive to ciprofloxacin than wild type strains. In addition, they demonstrate that replication restart is essential in response to ciprofloxacin and may play a role in tolerating the effects of resistance-conferring gyrase mutations. Accordingly, these findings establish that RecBC(D) and other components of double-stranded break repair or stalled replication fork rescue or repair pathways are required for the repair of DNA damage caused by ciprofloxacin. Accordingly, these studies indicate that RecBC(D) and RecA, as well as PriA, RecG, RuvB, and RuvC, are important targets in the treatment of both sensitive and resistant strains, and demonstrate that inhibitors of these polypeptides (or other polypeptides involved in double-stranded DNA break repair, replication restart, or fork repair) can be used to increase the drug sensitivity of and, ultimately, reduce viability or kill both sensitive and resistant strains.

### Example 2

### The Roles of Various Genes in Determining Sensitivity to Ciprofloxacin and the Ability to Evolve Resistance to Ciprofloxacin

With the isogenic loss of function strains in hand, mutation in response to ciprofloxacin (obtained from U.S. Biologicals) was determined using a protocol based on the Stressful Lifestyle Adaptive Mutation (SLAM) assay, as depicted in FIG. 3. Five colonies of each strain, selected from 30 ug/mL kan plates, were grown for 24 hours in LB at 37° C. Dilutions of each culture were made in duplicate and plated on LB plates to determine the number of viable cells.

To assay for mutation, 150 μL of each culture was plated twice on LB plates containing 35 ng/mL ciprofloxacin. Also, two 150 μL cultures from each strain were plated on five additional plates for use in ‘survival’ experiments (see below). The concentration of ciprofloxacin used was chosen based on trial experiments with the MG1655 parent strain which indicated that 35 ng/mL ciprofloxacin maximized mutation-dependent growth. Every twenty-four hours for thirteen days post-plating, colonies were counted and marked and up to 10 representative colonies per strain were stocked in 15% glycerol and stored at −80° C., for use in the reconstruction experiments (see below). Also, to determine the number of ciprofloxacin susceptible cells remaining on the plates, parallel ‘survival’ experiments were performed. The ‘survival’ experiment plates were treated exactly as the SLAM plates, except at specified time points, representative plates were sacrificed by excising all visible colonies, recovering the remaining agar in 9 mg/mL saline, and plating dilutions of the resulting solution on LB to determine the number of viable cells.

After thirteen days, a reconstruction experiment (Bull, H J, et al., Proc Natl Acad Sci USA (2001) 98:8334-8341; and Rosenberg, S M, (2001) Nat. Rev. Genet. 2:504-515) was performed to determine which of the resistant colonies isolated had evolved resistance via induced mutation after exposure to the antibiotic. The stocked colony suspensions isolated during the original experiment were used to inoculate 1 mL of LB and grown overnight at 37° C. The resulting cultures were then diluted and duplicate plated on LB and LB containing 35 ng/mL ciprofloxacin, and the time elapsed to colony formation was recorded and compared to the original experiment. Only those colonies that grew in a shorter time during the reconstruction experiment than in the original experiment were considered to have acquired an induced mutation, i.e., occurred after exposure to the antibiotic. Using the colony counts of induced mutants on the ciprofloxacin containing SLAM plates and the viable cell counts from the ‘survival’ experiments, an induced mutation rate was calculated per viable cell.

The data from these experiments are shown in Table 4. As indicated the frequency of mutation to ciprofloxacin resistance was found to be significantly reduced in several strains, including polBΔ (Pol II deletion strain); dinBΔ (Pol IV deletion strain); umuDCΔ (Pol V deletion stain), and lexA(lnd⁻) (which cannot under autocleavage and thus makes the strain uninducible). The largest effect from any single mutation was seen for the LexA(lnd⁻) strain which had a reduction of more than two orders of magnitude in the frequency of developing resistance to ciprofloxacin (the precise amount depending on the antibiotic concentration). The observed effect is remarkably large when considered in the context of clinical resistance. Clinically relevant high resistance requires multiple independent mutations. See Drlica, K, et al. Microbiol Mol. Biol. Rev. (1997) 61:377-392; Gibreel, A, et al. Antimicrob. Agents Chemother. (1998) 42:3276-3278; Kaatz, G W, Antimicrob Agents Chemother. (1993) 37:1086-1094; Yoshida, H, et al. J. Bacteriol., (1990) 172:6942-6949; Poole, K., Antimicrob. Agents Chemother. (2000) 44:2233-2241; Kern, W V, Antimicrob. Agents Chemother. (2000) 44:814-820; Fukuda, H, Antimicrob. Agents Chemother. (1998) 42:1917-1922, whereas resistance in these experiments requires a single mutation (in the gyrA gene, confirmed by sequencing).

TABLE 4
Strain growth, ciprofloxacin sensitivity, and mutation spectra

| | | | | | | | | Post-ciprofloxacin | | |
|---|---|---|---|---|---|---|---|---|---|---|
| | | ciprofloxacin | | | | | | Exposure | | |
| | | MIC | | | Exponential Growth | | | Day 5-13 Mutation | | |
| | Relative | (ng/ml) | | | Mutation Spectra | | | Spectra | | |
| | Doubling | WT | gyrA | gyrA | % | % Base | % | % | % Base | % |
| Strain | Time | gyrA | S83Δ | S83L | WT | Substitution | Codon Δ | WT | Substitution | Codon Δ |
| ΔlacZ | 1.0 (±0.01) | 35.0 | 250.0 | 450 | 16.7 | 83.3 | 0.0 | 22.2 | 61.2 | 16.7 |
| ΔpolB | 1.1 (±0.10) | 30.0 | 250.0 | 450 | 28.6 | 71.4 | 0.0 | 0.0 | 0.0 | 100.0 |
| ΔdinB | 1.0 (±0.03) | 35.0 | 250.0 | 450 | 16.7 | 83.3 | 0.0 | 0.0 | 0.0 | 100.0 |
| ΔumuDC | 1.0 (±0.01) | 35.0 | 250.0 | 450 | 25.0 | 75.0 | 0.0 | 33.3 | 0.0 | 66.7 |
| ΔpolB, ΔdinB | 1.0 (±0.12) | 25.0 | 250.0 | 450 | 50.0 | 50.0 | 0.0 | 83.3 | 0.0 | 16.7 |
| ΔpolB, | 1.1 (±0.19) | 25.0 | 250.0 | 450 | 66.7 | 33.3 | 0.0 | 0.0 | 0.0 | 100.0 |
| ΔumuDC | | | | | | | | | | |
| ΔdinB, | 1.1 (±0.08) | 35.0 | 250.0 | 450 | 16.7 | 83.3 | 0.0 | 33.3 | 0.0 | 66.7 |
| ΔumuDC | | | | | | | | | | |
| ΔpolB, | 1.2 (±0.17) | 25.0 | 250.0 | 450 | 42.9 | 57.1 | 0.0 | 0.0 | 0.0 | 100.0 |
| ΔdinB, | | | | | | | | | | |
| ΔumuDC | | | | | | | | | | |
| lexA (S119A) | 1.0 (±0.03) | 30.0 | 250.0 | 350 | 16.7 | 83.3 | 0.0 | 0.0 | 0.0 | 100.0 |
| ΔrecD | 1.0 (±0.10) | 35.0 | 250.0 | 350 | 0.0 | 100.0 | 0.0 | 0.0 | 80.0 | 20.0 |
| ΔrecA | 1.1 (±0.02) | 5.0 | | | | | | | | |
| ΔrecB | 1.1 (±0.04) | 7.5 | | | | | | | | |
| ΔrecG | 1.0 (±0.02) | 10.0 | | | | | | | | |
| ΔruvB | 1.1 (±0.14) | 10.0 | | | | | | | | |
| ΔruvC | 1.0 (±0.04) | 10.0 | | | | | | | | |
| ΔpriA | 1.1 (±0.04) | <1.0 | | | | | | | | |

The ΔlacZ strain was constructed as a control, and exhibited wild-type growth and mutation (±1.15-fold) in all cases. Other strains were constructed and characterized to examine the contribution of recombination and the SOS response to the survival in the presence of the antibiotic, and to the evolution of resistance.

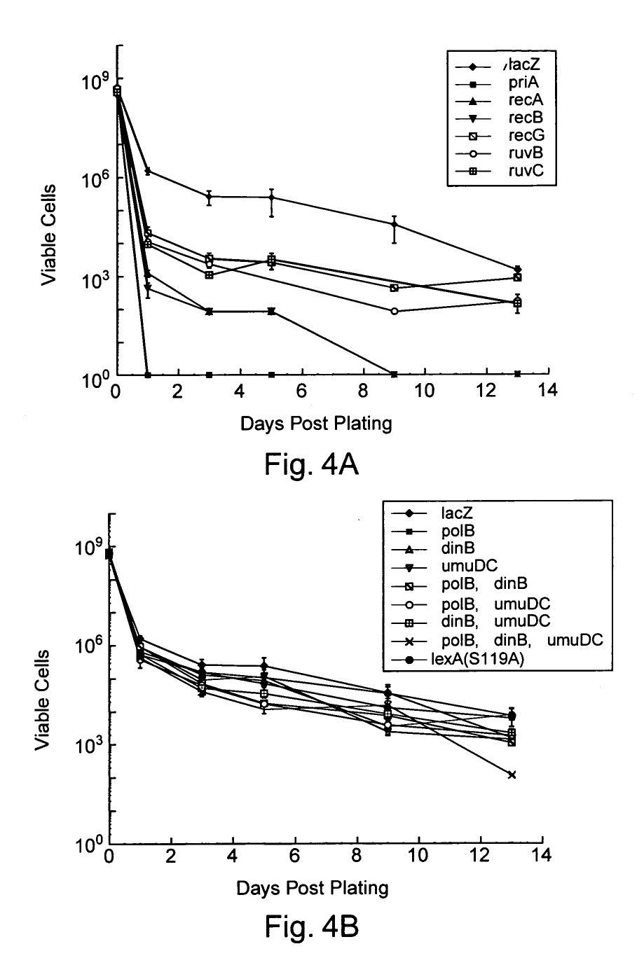

Given the apparent importance of recombination dependent replication restart to induced mutation at the lac allele, its role in response to ciprofloxacin was examined (FIG. 4). Deletion of priA, whose protein product facilitates replication restart after replication fork collapse by reloading replisome proteins, resulted in an extreme sensitivity to the antibiotic (MIC<1 ng/ml ciprofloxacin), implying that replication restart is required in response to the drug. This conclusion remains valid even in the presence of possible suppressor mutations (common in ΔpriA strains), because the strain remains hypersensitive to ciprofloxacin. recA and recB encode proteins required for recombination. The ΔrecA and ΔrecB strains exhibited nearly wild-type growth in the absence of ciprofloxacin, but were both hypersensitive to the antibiotic. The ΔrecG, ΔruvB, and ΔruvC strains, lacking the corresponding proteins involved in processing recombination intermediates, also showed no major growth defects in the absence of ciprofloxacin, and a high sensitivity to the drug, although not as great as the ΔrecA and ΔrecB strains. While the hypersensitivity to ciprofloxacin precludes determination of a post-exposure mutation rate in these strains (no resistant colonies could be isolated), it indicates that recombination-dependent replication restart becomes essential in the presence of ciprofloxacin, even at the low concentrations used in these experiments. In contrast, deletion of recD had no effect on the sensitivity to the antibiotic or the rate of mutation, implying that resectioning to a Chi sequence is not critical for repair of ciprofloxacin induced DNA damage. The lexA(S119A) strain showed virtually wild-type sensitivity to ciprofloxacin, implying that induction of the SOS response is not required as a response to the drug at this low concentration. However, the frequency with which bacteria evolved resistance to 35 ng/mL ciprofloxacin was reduced by approximately 100-fold in this strain (data not sown). These observations establish that RecA, RecB, RecG, RuvB, RuvC and PriA are attractive targets for the development of drugs that hypersensitize bacteria to ciprofloxacin, other quinolones, and other DNA damaging agents.

### Example 3

### Deletion of RecB Sensitizes both FQs and FQ^{R} Strains to Ciprofloxacin

To investigate the effect of recB mutation in FQ^{r} gyrA mutants, the recB gene was deleted from gyrA FQ^{r} mutants, and these strains were assayed for ciprofloxacin response (Table 5). The deletion of recB was carried out using P1-mediated transduction of a recB::Km^{r} allele into strains harboring gyrA FQ^{r} mutations, including gyrA-S83L in two different strain backgrounds, and gyrA-D87G.

Notably, it was demonstrated that deletion of recB from each of the gyrA FQ^{r} mutant strains significantly re-sensitized the strains to ciprofloxacin, 5- to 8-fold depending upon the strain background and the specific gyrA* mutation. In addition, deletion of recB from a wild-type FQ-sensitive strain sensitized the strain approximately 8-fold. Taken together, these results demonstrate that an inhibitor of RecB is an effective combination therapy with fluoroquinolone antibiotics against both FQ-sensitive as well as FQ-resistant infections.

TABLE 5
Ciproflaxocin MICs of ΔrecB gyrA* strains

ciprofloxacin
MIC (ng/ml)

| E. coli strain | recB+ | ΔrecB |
|---|---|---|
| MG1655 (gyrA+) | 25 | 3 |
| MG1655 gyrA-S83L | 500 | 100 |
| AB1157 gyrA-D87G | 200 | 25 |
| DM4100 gyrA-S83L | 400 | 50 |

Interestingly, it was found that the efficiency of general P1 transduction was approximately 10-fold lower using the ΔrecB strain as a P1 donor compared to control donors. Also, the efficiency of general P1 transduction into the gyrA FQ^{r} (gyrA*) mutant strains was approximately 20-fold less efficient compared to control recipients. The combination of these factors resulted in a greatly reduced efficiency of the ΔrecB allele into gyrA* backgrounds, but transductant colonies were still obtained.

Because of the very low efficiency of transduction observed, several steps were taken to confirm the genotype of each ΔrecB gyrA* strain. The presence of the ΔrecB allele was confirmed using three methods. First, attempts were made to PCR amplify the recB locus from the putative ΔrecB strains and recB+ controls. The recB+ locus was PCR amplified from all recB+ parental strains, but a product was not amplified from any of the putative ΔrecB strains. Second, the ΔrecB strains were tested in a P1 plaque assay. It was observed that rec+ strains are able to support P1 plaque formation, while rec-strains (including the ΔrecB strain) are not. Consistent with this observation, P1 was unable to form plaques on the putative ΔrecB transductants. Third, the Mitomycin C MIC of the putative ΔrecB transductants matched that of the parental ΔrecB strain (about 8-fold lower compared to recB+ strains).

The presence of the gyrA* FQ^{r} mutations was also confirmed by PCR amplification and sequencing of the quinolone resistance determining region (QRDR) from all strains. The genotypes matched expectations in all cases. The Cipro MICs of these ΔrecB gyrA* strains compared to the parental ΔrecB strain also suggested the presence of the gyra* mutations (Table 5).

### Example 4

### Temperature Sensitive recB and recC Mutants Exhibit an Increased Sensitivity to Ciprofloxacin

In order to further demonstrate the role of RecBC(D) in the maintenance of stable ciprofloxacin resistance, the biological consequences of abrogating RecBC(D) activity was examined in the context of strains that had evolved resistance to low (35 ng/ml) levels of ciprofloxacin.

Stressful lifestyle adaptive mutation (SLAM) assays were performed in strain SK119 containing a temperature sensitive RecB mutation (Kushner, S., J. Bacteriol. 1213 (1974)), essentially as described in Cirz et al. (pending publication) and depicted in FIG. 3. Briefly, 1×10⁷ cells from three separate cultures were spread on to each of 8 LB plates containing 35 ng/ml ciprofloxacin. The plates were incubated for five days at 30° C. During that time, six colonies were picked by excising a 3 mm plug from the agar plate that was resuspended in 1 ml of 15% glycerol. Ten microliters of this resuspension was streaked out on a fresh plate containing 35 ng/ml ciprofloxacin. A single colony was picked from each of these six ciprofloxacin resistant strains as well as the parental strain, and the MIC was measured as described (Cirz et al., pending publication).

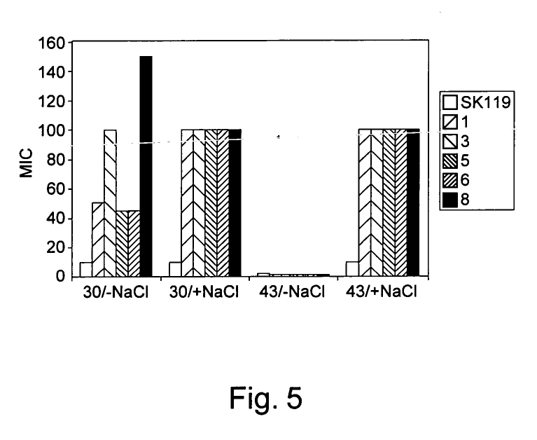

The MIC was examined under four different conditions: 30° C. and 43° C. in the presence or absence of NaCl. Media containing NaCl consisted of 10 g bacto tryptone, 5 g yeast extract, 5 g NaCl in 1 L of H₂O at pH 7.0. Media lacking NaCl was of the identical composition, but with no added NaCl. These four conditions were examined, since the temperature sensitive phenotype is only observed under conditions of low salt (Kushner, S., J. Bacteriol. p 1213 (1974)). As indicated in FIG. 5, the MICs of the strains ranged from 50-150 ng/ml at the permissive temperature and at the non-permissive temperature in the presence of high salt. However, at the non-permissive temperature and low salt, the MICs of all six strains was dramatically shifted to 1 ng/ml.

These studies demonstrate that these strains are highly dependent on the presence of RecB activity to tolerate otherwise sublethal concentrations of ciprofloxacin. In addition, these data further establish that RecBC(D) and other components of the homologous recombination or recombination dependent DNA replication pathways, are required for the maintenance of stable ciprofloxacin resistance and, thus, represent important targets in the development of new drugs for the treatment of resistant strains.

### Example 5

### Target-Based Method of Identifying Small Molecule Inhibitors of RecBC or RecBCD

Small molecule inhibitors of RecBC(D) are identified by screening a library of chemical compounds for their ability to bind recombinant RecBC(D) (“RecBC(D)” indicates that one can screen either RecBC or RecBCD in any of the indicated steps) using the Automated Ligand Identification System (ALIS), essentially as described in U.S. Pat. Nos. 6,721,665, 6,714,875, 6,694,267, 6,691,046, 6,581,013, 6,207,861, and 6,147,344. ALIS is a high throughput technique for the identification of small molecules that bind to proteins of interest.

Using this technique, recombinantly produced and purified RecBC(D) is combined with 5,000 pools of compounds, each pool containing approximately 5,000 compounds, each compound having a precise molecular structure that can be determined based upon its mass (and knowledge of the compounds present in the library). The RecBC(D) proteins and the compounds are mixed together for 30 minutes at room temperature to permit binding. The mixture is then rapidly cooled to trap bound complexes and subjected to rapid size exclusion chromatography (SEC). Small molecules that bind tightly to RecBC(D) and are co-excluded with RecBC(D) during SEC are then subjected to mass spectroscopic analysis to determine their masses. The mass of each compound is then used to determine its molecular structure. The corresponding structure is then resynthesized, and its ability to bind RecBC(D) is confirmed in a binding assay.

Confirmed binders are subsequently tested in a helicase assay to identify inhibitors of RecBC(D)-mediated helicase activity (Nature. 2003 Jun. 19; 423(6942):889-93; Eggleston NAR 24:1179-1186, 1996). The RecBCD complex encodes both a 5′-3′ (RecD) and a 3′-5′ (RecBC) helicase. recD mutants are still recombination proficient and are not hypersensitized to FQs (Example 1). In contrast, recB mutants are hypersensitized to FQs (Example 1) and are deficient in HR. Thus, in certain embodiments, inhibitors of RecBC are desired. The helicase assay is performed using a purified RecBC helicase fraction or a RecBC(D) (recD K177Q) mutant, as the K177Q mutation has been shown to disable the helicase activity of RecD. Compounds identified as inhibitors of the RecBC helicase activity are subjected to SAR to identifiy structurally diverse analogs with a range of potencies.

Compound series identified as binding RecBC(D) are tested for their ability to increase ciprofloxacin sensitivity in wild type gyrA and mutant gyrA(S83L) strains of E. coli MG 1655. Each of these strains is treated with 35 ng/ml of ciprofloxacin in the presence or absence of various amounts of a compound identified as binding RecBC(D), and the MIC is determined. Compounds that result in lower MIC values are identified as compounds that inhibit RecB activity. To avoid false positives due to low permeability into cells or efficient elimination of compounds by efflux pumps, these assays are performed using strains and conditions that favor permeability into cells and that reduce the activity of efflux pumps by, for example, mutation or by the addition of inhibitors.

Analogs and derivatives of these compounds are synthesized using various techniques known in the art and further tested for their ability to reduce MIC value using an in vivo thigh challenge model, essentially as described in Andes and Craig, Antimicrob Agents Chemother. 46:1665-70 (2002)). Briefly, six week old, specific pathogen-free, female CD-1 mice are rendered neutropenic (neutrophil counts<100/mm³) by injecting 150 mg/kg cyclophosphamide intraperitoneally four days before infection and 100 mg/kg cyclophosphamide 24 hours before infection. Mueller-Hinton (MH) broth cultures inoculated from freshly plated bacteria are grown to logarithmic phase (OD580 of approximately 0.3) and diluted 1:10 in MH broth. Thigh infections are produced by injecting 0.1 ml volumes of the diluted broth cultures into halothane-anesthetized mice. Beginning two hours after infection (defined as time zero), mice are administered subcutaneous injections of either 0.5 mg/kg ciprofloxacin in the presence or absence of various amounts of a compounds being tested every 12 hours for three days. At each time point tested, both thighs from two sacrificed animals are removed and homogenized. Serial dilutions of thigh homogenates are plated on MH agar and MH agar containing about 10-80 ng/ml ciprofloxacin. After 24 hours, visible colonies are counted and excised from the plates to determine the total number of viable, ciprofloxacin-resistant cells. The remaining agar is homogenized in saline, and serial dilutions are plated in duplicate on LB agar to determine the total number of viable, ciprofloxacin-sensitive cells present. Compounds resulting in a reduced number of viable, ciprofloxacin-resistant or sensitive cells are identified as compounds that inhibit the activity of RecBC and are useful in treating ciprofloxacin resistant strains.

### Example 6

### Activity-Based Methods of Identifying Inhibitors

Small molecule inhibitors of RecB are identified based upon their ability to inhibit RecBC(D) ATPase activity. An in vitro assay for recombinant RecBC(D) ATPase activity is used to screen a library of small molecules for their ability to inhibit RecBC(D) activity.

In brief, His-tagged RecC or wild type RecC and wild-type RecB and RecD polypeptides are coexpressed in E. coli from bacterial expression vectors, such that the proteins form a native heterotrimer. Alternatively, RecBC(D) or mutant complexes such as RecBC(D)(K177Q) are expressed and purified in order to focus the assay on a particular activity of interest. These heterdimers or heterotrimers are then purified using a Ni-affinity columns or under other well-established conditions that maintain the heterotrimer. Recombinant expression of RecBC(D) has previously been described in Amundsen, S. K. et al., PNAS: 7399-7404 (2000) and Dillingham, M. S. et al., Nature: 893-897 (2003).

RecBC(D) ATPase activity is determined by measuring ATP hydrolysis using ³²P-ATP coupled to NADH oxidation, basically as described in Nucl. Acid. Res. 28:2324 (2000). Essentially, purified His-tagged RecBCD is incubated with ³²P-ATP, dsDNA, (NH₄)₂MoO₄, and malachite green. ATP hydrolysis is then determined based upon NADH oxidation, as measured at 660 nm absorbance.

To identify an inhibitor of RecBC(D) ATPase activity, a library of small molecules is screened using the NADH oxidation coupled ATP hydrolysis assay in a high throughput format, using 96-well plates. Recombinant RecBC(D) is placed into each well with the appropriate substrates. In addition, pools of different small molecules are added to each well (except a control well, to which no small molecules are added), and NADH oxidation is measured. Wells exhibiting decreased NADH oxidation are identified as containing a small molecule that inhibits RecBC(D) ATPase activity. Small molecules that were included in these well are then rescreened individually for their ability to inhibit RecBC(D) ATPase activity.

Compounds identified as inhibiting RecBC(D) ATPase activity are then tested for their ability to increase ciprofloxacin sensitivity in wild type gyrA and mutant gyrA(S83L) strains of E. coli. Each of these strains is treated with 35 ng/ml of ciprofloxacin in the presence or absence of various amounts of a compound, and the MIC is determined. Compounds that result in lower MIC values are identified as compound that increase ciprofloxacin sensitivity.

Analogs and derivatives of these compounds are synthesized using various techniques known in the art and further tested for their ability to reduce MIC value and in an in vivo thigh challenge model, as described above.

These methods are also used to obtain inhibitors of homologues of RecBCD such as the AddAB gene products in Bacillus subtilis and Bacillus anthracis and the RexAB proteins of Streptococcus pneumoniae.

### Example 7

### Inhibition of RecBC Reduces the Viability of Ciprofloxacin Resistant Y. pestis

The E. coli data presented in the previous Examples suggests that mutations in the Yersinia pestis topoisomerases gyrA, and parC that confer resistance to fluoroquinolones in clinical isolates will also be lethal to Y. pestis when combined with inhibition of the recombination machinery encoded by RecBC(D). This hypothesis will be confirmed by constructing a mutant Y. pestis strain, harboring a deletion of RecBC(D) in combination with FQ^{r} mutations, and testing that strain for survival rate and ciprofloxacin MIC. According to the hypothesis, deletion of recBC in the FQ^{r} strain will lead to cell death and a significant increase in ciprofloxacin effectiveness as measured by MIC. This study will directly address the hypothesis that after Y. pestis evolves FQ^{r}, there is an absolute dependence on RecBC(D)-mediated homologous recombination and that inhibitors of RecBC(D) in combination with ciprofloxacin are an effective antibiotic against resistant Y. pestis.

It is possible that deletion of RecBC(D) from FQ^{r} mutant Y. pestis will be so deleterious that direct construction of the double mutant strain is impossible. In this case, an alternate strategy will be employed to demonstrate synthetic lethality (combination lethality) of these mutations using a plasmid stability approach. Briefly, a plasmid (with an unstable replicon) expressing functional RecBC will be introduced in FQ^{r} mutant Y. pestis strains, and subsequently the RecBC gene will then be deleted from the chromosome. If FQ^{r} mutants are synthetic lethal with loss of RecBC function, the unstable plasmid will be retained in the double mutant strain.

Analogous approaches will be used to test the interaction between mutations in RecBC (or its homologues) and gyrA in other species of interest such as Bacillus anthracis.

### Example 8

### High Throughput Screening Methods to Identify Small Molecule Inhibitors of RecBC(D)

Small molecule inhibitors of RecB helicase are identified using a high throughput screening assay of a library of small molecules. Briefly, RecBC(D) proteins are recombinantly expressed and purified as previously described. These recombinant proteins are exposed to various small molecules in multi-well plates, and their helicase activity is determined by addition of a dsDNA substrate, a dsDNA-specific dye, and ATP. In the absence of inhibitor, the RecBC will unwind the dsDNA, producing ssDNA and a concurrent decrease in the fluorescent signal. The fluorescent signal is monitored using a standard plate reader, either in real time or at a predetermined end point. Accordingly, small molecules that inhibit RecBC helicase activity are identified as resulting in increased fluorescence.

A single point mutation in gyrA, S83L, confers high levels of resistance to ciprofloxacin in E. coli. A strain possessing this mutation, in addition to higher than normal membrane permeability, will be used to identify small molecules that sensitize bacteria to the damaging effects of ciprofloxacin. This is indicative of compounds that inhibit RecBC, thereby preventing the DNA repair processes necessitated by the mutant gyrase and ciprofloxacin. The permeable gyrAS83L strain is grown in multiwell plates containing ciprofloxacin at a dose that is just below the minimum inhibitory concentration for this strain. In the absence of an inhibitor, the bacteria grow, producing optical density that is readily observed using an absorbance plate reader to monitor light scattering at 600 nM. Small molecule libraries are added to these plates, which cause a decrease in growth rate, if they target RecBC, and a corresponding decrease in OD₆₀₀. Similar screens are carried out in alternate strain backgrounds, such as wild type E. coli or other resistant strains, to account for variations in membrane permeability or pumps that prevent accumulation of inhibitors.

Compounds identified in either of the above high throughput screens are further characterized via a variety of methods. The MIC of the putative RecBC(D) inhibitor is determined by observing growth over a range of compound concentrations in the presence and absence of ciprofloxacin. A strain analogous to that used in screening, but lacking the recB gene, is used to test for specificity of the ciprofloxacin sensitization effect, thereby ruling out non-specific toxicity of the compound. Conversely, the amplification of RecB activity via insertion of additional copies of the gene or a stronger promoter should increase the amount of compound required to observe death at an otherwise sub-lethal concentration of ciprofloxacin. Similar methods can be used to test whether the mechanism of action involves other logical targets in this pathway, including RecA, RecG, PriA, RuvB or RuvC.

RecBC(D) function is assessed using any of several different genetic tests in vivo. First, the ability to transfer a genetic marker from a Hfr (high frequency of recombination) donor strain to a recipient is tested in the presence of inhibitor. Recombination defective strains are severely impaired for F mediated transfer (˜500-fold) because the process requires homologous recombination. Therefore, this assay allows a large dynamic range to measure recB inhibition. Second, T4 bacteriophage mutated for gene2 (T4 gp2−) cannot replicate on wildtype E. coli due to digestion of the viral DNA upon injection, but can efficiently replicate in recBC or recD defective strains. RecB inhibition is, therefore, assayed by measuring the ability of T4 gp2− phage to replicate on E. coli treated with inhibitors. T4 gp2− phage replication is monitored by following the decrease in OD600 due to bacteria lysis in liquid culture, or by using a fluorogenic or colorimetric marker on engineered T4 phage. Finally, infection of E. coli by bacteriophage P1 requires functional RecBC enzyme, as indicated by the observation that P1 phage cannot form plaques on RecBC mutant E. coli. Thus, RecBC inhibition is assayed by measuring the ability of P1 phage to replicate on E. coli, using similar readouts as those described for T4 gp2− above.

### Example 9

### An Inhibitor of RecBC(D) Increases the Ciprofloxacin Sensitivity of Resistant E. coli

To test the model that inhibition of RecB would significantly sensitize already resistant bacteria to ciprofloxacin, the effect of the λ_{gam} protein was examined. This protein is used by λ phage to inhibit E. coli RecB in order to prevent its cleavage of the phage genome during infection. To construct an arabinose-inducible expression system for the study of gam overexpression in E. coli, the gam gene from λ phage was amplified by PCR from strain PS6275 and cloned into the NdeI/XhoI sites of vector pBadAss resulting in vector pRTC0045. pRTC0045 and its corresponding empty vector control (pBadAss) were each transformed into E. coli strains RTC0086, RTC0110 and RTC0013 resulting in a total of 6 strains (Table 6).

TABLE 6
E. coli strains for lambda gam overexpression

| Strain | Relevant Genotype | Source |
|---|---|---|
| RTC0141 | ΔaraA::Gm^{R}, +pBadAss | Transform RTC0086 + pBadAss |
| RTC0142 | ΔaraA::Gm^{R}, | Transform RTC0086 + pRTC0045 |
| | +pRTC0045 | |
| RTC0143 | ΔaraA::Gm^{R}, | Transform RTC0110 + pBadAss |
| | gyrA(S83L), +pBadAss | |
| RTC0144 | ΔaraA::Gm^{R}, | Transform RTC0110 + pRTC0045 |
| | gyrA(S83L), | |
| | +pRTC0045 | |
| RTC0147 | ΔaraA::Gm^{R}, | Transform RTC0013 + pBadAss |
| | ΔrecB::Km^{R}, +pBadAss | |
| RTC0148 | ΔaraA::Gm^{R}, | Transform RTC0013 + pRTC0045 |
| | ΔrecB::Km^{R}, | |
| | +pRTC0045 | |

To construct an arabinose-inducible expression system for the study of gam overexpression in P. aeruginosa, the araC gene, pBad promoter, gam gene or empty insert control, and rnnB terminator regions from pBadAss and pRTC0045 were amplified by PCR and each cloned into the ApaI/XbaI site of vector pBBR1MCS-4 resulting in vectors pRTC0049 and pRTC0050, respectively. Both vectors were transformed into E. coli strain S17-1 and moved into P. aeruginosa strains ATCC 27853, RTC1013 and RTC1012 by conjugal mating resulting in a total of 6 strains (Table 7).

TABLE 7
P. aeruginosa strains for lambda gam overexpression

| Strain | Relevant Genotype | Source |
|---|---|---|
| RTC1014 | +pRTC0049 | Mate ATCC 27853 + pRTC0049 |
| RTC1017 | +pRTC0050 | Mate ATCC 27853 + pRTC0050 |
| RTC1015 | gyrA(S83I), +pRTC0049 | Mate RTC1013 + pRTC0049 |
| RTC1018 | gyrA(S83I), +pRTC0050 | Mate RTC1013 + pRTC0050 |
| RTC1016 | ΔrecB::Gm^{R}, +pRTC0049 | Mate RTC1012 + pRTC0049 |
| RTC1019 | ΔrecB::Gm^{R}, +pRTC0050 | Mate RTC1012 + pRTC0050 |

To determine the effect of gam overexpression on ciprofloxacin efficacy in E. coli, for each of the 6 strains an overnight culture was grown in Luria Broth (LB)+100 μg/ml ampicillin. 96-well plates containing 150 μl LB+100 μg/ml ampicillin, +/−ciprofloxacin, +/−arabinose were inoculated with approximately 5×10⁴ CFU of starting bacteria. Plates were covered with sterile, breathable filters and incubated for 18.5 hours at 37° C. After incubation, the OD₆₅₀ was read in a 96-well plate reader and used to determine ciprofloxacin IC50s, IC90s, and MICs in the presence and absence of gam (Table 8).

TABLE 8
Effect of gam expression on ciprofloxacin efficacy in E. coli

| | No Arabinose | | | 0.25% Arabinose | | |
|---|---|---|---|---|---|---|
| Strain | MIC (ng/ml) | IC50 | IC90 | MIC | IC50 | IC90 |
| RTC0141 | 35.0 | 15.3 | 28.1 | 35.0 | 18.5 | 32.8 |
| RTC0142 | 35.0 | 15.0 | 25.6 | 15.0 | 4.10 | 8.20 |
| RTC0143 | 750 | 257 | 515 | 750 | 428 | 725 |
| RTC0144 | 750 | 268 | 510 | 200 | 54.1 | 170 |
| RTC0147 | 15.0 | 2.3 | 4.6 | 15.0 | 2.8 | 5.1 |
| RTC0148 | 15.0 | 2.5 | 4.8 | 15.0 | 3.7 | 6.9 |

Overexpression of lambda gam resulted in an approximately 5-8 fold decrease in the ciprofloxacin IC50 for both wild-type E. coli (strain RTC0142) and E. coli containing a gyrA(S83L) mutation (strain RTC0144) relative to their empty vector control strains (strain RTC0141 and RTC0143, respectively). In contrast, overexpression of lambda gam in a strain lacking the RecB target (strain RTC0148) had no effect on the strain's ciprofloxacin sensitivity relative to the control, empty vector strain (RTC0147).

To determine the effect of lambda gam overexpression on ciprofloxacin efficacy in P. aeruginosa, for each of the 6 strains, an overnight culture was grow in LB+350 μg/ml carbenicillin. 96-well plates containing 150 μl LB+350 μg/ml carbenicillin, +/−ciprofloxacin, +/−arabinose were inoculated with approximately 5×10⁴ CFU of starting bacteria. Plates were covered with sterile, breathable filters and incubated for 18.5 hours at 37 C. After incubation, the OD650 was read in a 96-well plate reader and used to determine ciprofloxacin IC50s, IC90s and MICs in the presence and absence of gam (Table 9).

TABLE 9
Effect of gam expression on ciprofloxacin efficacy in P. aeruginosa

| | No Arabinose | | | 2.0% Arabinose | | |
|---|---|---|---|---|---|---|
| Strain | MIC (ng/ml) | IC50 | IC90 | MIC | IC50 | IC90 |
| RTC1014 | 400 | 84.5 | 177 | 400 | 81.7 | 172 |
| RTC1017 | 400 | 93.2 | 182 | 100 | 61.8 | 98.8 |
| RTC1015 | 11000 | 4100 | 7530 | 11000 | 2760 | 6640 |
| RTC1018 | 11000 | 3694 | 7122 | 5000 | 1630 | 3206 |
| RTC1016 | 25.0 | 9.2 | 19.7 | 25.0 | 12.9 | 22.7 |
| RTC1019 | 25.0 | 11.0 | 21.2 | 25.0 | 12.8 | 22.7 |

In P. aeruginosa, overexpression of lambda gam resulted in an approximately 2-4 fold decrease in the ciprofloxacin MIC for both wild-type P. aeruginosa (strain RTC1017) and P. aeruginosa containing a gyrA(S831) mutation (strain RTC1018) relative to their empty vector control strains (strain RTC1014 and RTC1015, respectively). In contrast, overexpression of lambda gam in a strain lacking the RecB target (strain RTC1019) had no effect on the strain's ciprofloxacin sensitivity relative to the control, empty vector strain (RTC1016).

### Example 10

### An Inhibitor of Replication Restart Increases the Ciprofloxacin Sensitivity of Wild Type and FQ Resistant E. Coli

One consequence of inhibition of type II topoisomerases by FQs is stalling of replication forks when they encounter the FQ:topoisomerase:DNA complex. These stalled forks must be repaired by recombination dependent fork repair. An essential step in this process is replication restart that is primed by the primosome. Therefore, inhibitors of the primosome hypersensitize cells to FQ, based on preventing repair of the stalled fork. The primosome consists of DnaG primase, DnaB helicase, PriA, PriB, PriC, DnaC and DnaT. Inhibitors that prevent the formation of a functional primosome will hypersensitize cells to FQ and other agents such rifampin and its analogs that give rise to blocked replication forks (stalled transcription complexes in the case of rifampin), based on preventing repair of the stalled forks.

Inhibitors are identified using target based screening, essentially as outlined in Example 3, but using components of the primosome as the target. Inhibitors are also identified by HTS activity based screens, such as replication restart on ssDNA templates such as φX174 DNA (http://www.sbsonline.org/sbscon/2004/posters/040629170352.htm) or other gram positive derived substrates (http://www.sbsonline.org/sbscon/2004/post/posters/040629165056.htm). Identified inhibitors are subsequently tested for their ability to hypersensitize bacteria to FQ and other DNA damaging agents.

### Example 11

### Discovery of Additional Targets That Hypersensitize Wild Type and FQ Resistant Bacteria to FQs

E. coli are used as a platform to screen for and validate additional targets that sensitize wild-type and FQ^{r} bacteria to ciprofloxacin. Such targets are genes which, when mutated, are synthetic lethal (lethal in combination) with FQ^{r} conferring mutations in gyrA and/or parC, re-sensitize FQ^{r} mutants to ciprofloxacin, or hypersensitize wild-type bacteria to ciprofloxacin. Once identified, inhibitors of the proteins encoded by these genes are developed to use in combination therapies with FQ antibiotics.

To identify genes that are synthetic lethal with FQ^{r} gyrA mutants, an unstable plasmid expressing functional gyrA and a colorimetric marker (e.g., LacZ) are introduced into a FQ^{r} gyrA mutant strain also harboring a mobilizable transposable element. The transposon is mobilized to randomly disrupt genes across the entire genome. If the transposon disrupts a gene that is required for FQ^{r} gyrA mutants to survive, the plasmid expressing wild-type gyrA protein become essential and stable, as judged by a colony sectoring assay for LacZ (Bernhart et al, Mol. Microbiol. 52:1255-1269, 2004). Alternatively, genes synthetic lethal with FQ^{r} gyrA mutants are identified using a transposable element harboring an outward facing, inducible transcription promoter (Judson. N, Mekalanos J J. TnAraOut, a transposon-based approach to identify and characterize essential bacterial genes, Nat Biotechnol. 2000 July; 18(7):740-5. PMID: 10888841). As above, this transposon is mobilized in a FQ^{r} gyrA mutant strain in the presence of small amounts of inducer, and synthetic lethal mutations are identified transposon insertions conferring slow growth (small colonies) in the presence of low inducer levels but no growth in the absence of inducer. The genes disrupted in these synthetic lethal mutations are identified by sequencing the genomic regions flanking the transposon insert.

To identify targets that re-sensitize FQ^{r} mutants to ciprofloxacin, the mutant pool described above is screened for hypersensitivity to ciproflaxin. Also, to identify targets that hypersensitize wild-type bacteria to ciprofloxacin, the transposons described above are mobilized in a wild-type background, and the mutant pool is screened for hypersensitivity to ciprofloxacin.

### Example 12

### Agents That Hypersensitize Mammalian Cells to DNA Damaging Agents

Nonhomologous end joining (NHEJ) or nonhomologous recombination (NHR) are major mechanisms for repair of DSB in mammals. This pathway generally repairs DSB by performing a microhomology search for regions with microhomology (about 3 to 10 bases) to a DSB and by repairing the lesion in a NHR reaction (Rathmell and Chu, DNA Double-Strand Break Repair, Chapter 16 of Nickoloff, J. A. and Hoekstra, M. F. in DNA Damage and Repair, Humana Press, Totowa, N.J., 1998). Major targets in this pathway are DNA protein kinase (DNA-PK), the Ku70 and Ku86 proteins, and the XRCC4 protein. The Ku proteins form a heterodimeric helicase that binds with high affinity to double stranded ends of DNA and recruits DNA-PK. Subsequently, Ku unwinds the DNA and promotes repair either by homology dependent or homology independent pathways (Rathmell and Chu, DNA Double-Strand Break Repair, Chapter 16 of Nickoloff, J. A. and Hoekstra, M. F. in DNA Damage and Repair, Humana Press, Totowa, N.J., 1998). Cells deficient in XRCC4, Ku86, or DNA-PK are hypersensitive to ionizing radiation. As this is a major pathway for DSB repair in mammals, inhibitors of proteins this pathway are predicted to hypersensitize cells to DNA damaging agents that cause DSB. Therefore, inhibitors of the Ku proteins (Ku70 and Ku86), DNA-PK, or XRCC4 should sensitize mammalian cells to DNA damaging agents and, thus, should be valuable drugs in combination with treatment regimes, such as treatment with chemotherapeutics or ionizing radiation, that generate DNA damage.

In order to identify inhibitors of Ku70, Ku86, DNA-PK, and XRCC4. the methods described in Example 5 are used essentially as described to identify binders and inhibitors of these proteins. A non-homologous end joining assay such as that developed by Chu (EMBO J. 2002 Jun. 17; 21(12):3192-200.) is used to screen for functional inhibitors of this reaction in a cell free assay. This is followed by screens in mammalian cells for analogues that hypersensitize cells to radiation and chemotherapeutic DNA damaging agents.

### Example 13

### Deletion of the recB Gene Sensitizes Multiple Bacterial Species to Ciprofloxacin

To confirm that the ciprofloxacin sensitization effect of a recB mutation in E. coli extends to other bacteria, the recB gene was deleted from a variety of bacterial species, and the resulting strains were assayed for sensitivity to ciprofloxacin. Deletion of recB was carried out using standard techniques in E. coli (ATCC25922), K. pneumoniae (ATCC43816), P. aeruginosa (ATCC27853), B. anthracis (Sterne), and S. aureus (NARSA77). The genomic structures of the knockouts were confirmed by PCR. The ciproflaxacin MIC was determined in both wild type and recB mutant strains (Table 10).

TABLE 10
Ciprofloxacin MICs of ΔrecB strains

| | MIC wt | MIC KO |
|---|---|---|
| Species | (ng/ml) | (ng/ml) |
| E. coli | 35 | 5 |
| ATCC25922 | | |
| K. Pneumo. | 35 | 8 |
| ATCC43816 | | |
| P. aeruginosa | 400 | 100 |
| ATCC27853 | | |
| B. anthracis | 50 | 3 |
| Sterne | | |
| S. aureus | 200 | 40* |
| NARSA77 | | |

*rexAB; agar MIC

Deletion of recB from each of the bacterial species examined significantly sensitized the strains to ciprofloxacin, 4- to 16-fold depending upon the particular species. These results demonstrate that recB plays an important role in bacterial sensitivity to fluoroquine antibiotics, including ciprofloxacin, and indicate that an inhibitor of RecB is an effective combination treatment with fluoroquine antibiotics for the treatment of a broad range of bacterial infections.

### Example 14

### Deletion of recB Increases Ciprofloxacin Sensitivity of Klebsiella Pneumoniae in an Animal Model

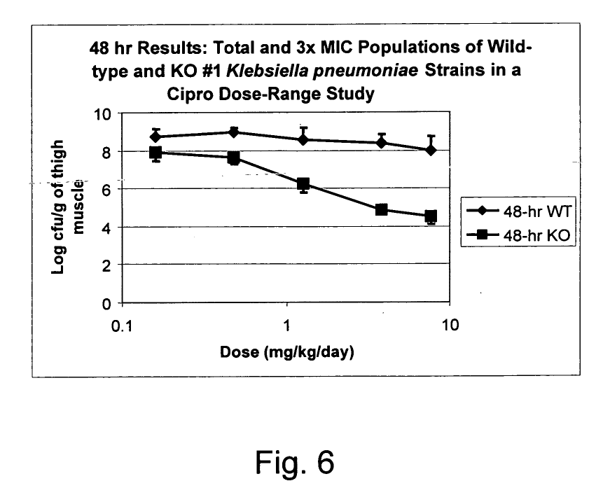

Ciprofloxacin dose response studies were performed using an immunocompetent murine thigh infection model of K. pneumoniae infection, in order to examine the effect of recB deletion. The thighs of mice were inoculated with 1-3×10⁷ CFU of strains of wild type or ΔrecB K. pneumoniae, and the mice were then treated at t=0 and 24 hours with doses ranging from 0.064-15 mg of ciprofloxacin per kg of body weight per day, with the dose fractionated for dosing every 24 h. Levels of bacteria in the thighs were measured by microbiologic assay at t=−2 and 0 hr for animals treated with saline and at 24 and 48 hours for animals treated either with saline or ciprofloxacin. Bio-fitness was measured at −2, 0, 24, and 48 hours. An exemplary graph of log CFU/gm of thigh at the 48 hour endpoint vs cipro dose (mg/kg/day) is shown in FIG. 6.

As is apparent from the graph in FIG. 6, there is a significantly greater reduction in CFU for the recB mutants relative to the wild type strain at every dose of cipro tested. This indicates that the sensitivity to ciprofloxacin that we see in vitro is also manifested as increased sensitivity to ciprofloxacin therapy in vivo. These studies indicate that deletion of recB results in enhanced ciprofloxacin sensitivity and more effective killing of K. pneumoniae in vivo, and demonstrate that inhibitors of the RecB helicase may be effectively used in combination with fluoroquinolones to treat K. pneumoniae and other bacterial infections. Furthermore, the enhanced killing of recB mutants as compared to wild type K. pneumoniae suggests that such a combination treatment will result in a faster cure and better efficacy for difficult to treat infections.

### Example 15

### Identification of RecB Inhibitors That Sensitize Cells to Ciprofloxacin

High throughput screening methods were utilized to identify small molecule inhibitors of RecBC(D) that enhance the sensitivity of bacteria to ciprofloxacin. Briefly, a library containing approximately 110,000 synthetic compounds was selected from a potential library of 650,000 compounds (Discovery Partners International, San Diego, Calif.). These compounds were screened in multi-well plates containing membrane permeabilized E. coli grown in the presence of approximately 0.5× the minimal inhibitory concentration (MIC) of ciprofloxacin. Enhanced permeability was engineered with the use of a hypomorphic allele of lpxA (an essential gene, but quantitative reduction in the amount of lipid A produced by the cell with the hypomorphic allele significantly compromises the outer membrane, resulting in increased permeability to small molecules). Active compounds were subsequently re-assayed plus or minus ciprofloxacin (to distinguish antibiotics from ciprofloxacin sensitizing agents) and in an isogenic strain containing a deletion in rep, a non-essential helicase which has been shown to be synthetically lethal with recB or priA mutations. Active compounds that passed these filters were subsequently tested for their ability to kill E. coil K12 MG1655 Δrep (in order to assess their ability to enter E. coli with wild type permeability).

The initial screen identified approximately 40 compounds exhibiting ciprofloxacin sensitization, as determined by measuring both the 0.5× and 0.1×MICs. These compounds represented multiple structural scaffolds. While certain compounds displayed reduced or low permeability into bacteria, all of these compounds were able to enhance the sensitivity of bacteria to ciprofloxacin under conditions facilitating entry into the cell.

A structure-based search was performed on the remaining 640,000 molecules in the library to identify analogs having at least 70% Tanemoto similarity, and this resulted in 1052 additional compounds, permitting expansion of active scaffolds with analogs having similar structural and chemical properties. These compounds were put through the screening cascade outlined above. Numerous compounds were identified, and several discrete scaffold structures were revealed, including those represented by compounds of Formulas I, II, and III, as described further below.

One compound identified is shown below as Formula Ia, which falls within the scaffold shown generically as Ib, wherein R is hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido. 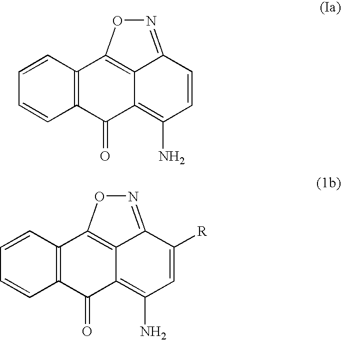
The compound of Formula Ia enhanced the sensitivity of E. coli to ciprofloxacin, providing a 10×MIC shift in ciprofloxacin responsiveness at 25 μM ciprofloxacin. Additional analogs were also identified that enhanced sensitivity to ciprofloxacin.

Another scaffold structure identified is represented generically in Formula IIa, wherein either A is nitrogen, and the other A is carbon. Specific compounds identified having this scaffold scructure are shown in Formulas IIb and IIc. 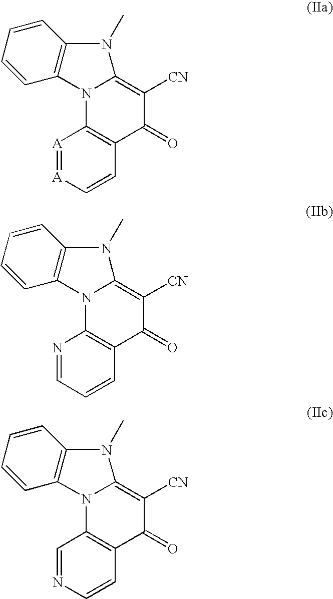
Each of these compounds enhanced the activity of ciprofloxacin at 25 μM.

Another scaffold identified during the screens is represented by the specific identified compound shown in Formula III. Compounds in this scaffold also exhibited enhanced ciprofloxacin sensitivity.

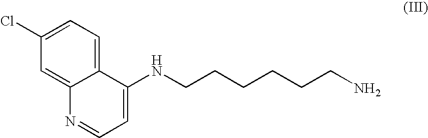

The results of the screen described above demonstrate the identification of a variety of structurally distinct compounds that sensitize cells to ciprofloxacin, consistent with these compounds being inhibitors of either recB or priA. These studies establish that the methods of the present invention can be successfully used to identify compounds that enhance the sensitivity of bacteria to fluoroquinolones. In addition, they demonstrate that a variety of chemical compounds having very different structural scaffolds share the functional characteristic of enhancing sensitivity of bacteria to fluoroquinolones.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

What is claimed is:

1. A method of identifying an agent that sensitizes a microorganism or cell to an antimicrobial or cytotoxic compound, the method comprising:
(a) screening one or more candidate agents for their ability to bind or inhibit an activity of a polypeptide associated with double-stranded DNA break repair or stalled replication fork rescue or repair; and
(b) identifying a candidate agent identified in step (a) that sensitizes a microorganism or cell to an antimicrobial or cytotoxic compound,
thereby identifying an agent that sensitizes a microorganism or cell to an antimicrobial or cytotoxic compound.

2. The method of claim 1, further comprising:
(c) producing a derivative or analog of the agent identified in step (b); and
(d) determining whether said derivative or analog enhances the sensitivity of the microorganism or cell to the antimicrobial or cytotoxic compound.

3. The method of claim 1, wherein said polypeptide is associated with homologous recombination, RecBC(D)-mediated homologous recombination, RecFOR-mediated homologous recombination, non-homologous recombination, non-homologous end joining, recombination-dependent replication fork repair, and/or primosome reassembly

4. The method of claim 1, wherein said polypeptide is selected from the group consisting of: RecB, RecA, PriA, DNA-PK, Ku70, and Ku86.

5. The method of claim 1, wherein said compound is a fluoroquinolone or a topoisomerase poison.

6. The method of claim 1, wherein said screening of step (a) is performed using a mutant strain of a microorganism.

7. A method of identifying a compound that enhances the sensitivity of a microorganism or cell to an antimicrobial or cytotoxic agent, the method comprising:
(a) contacting a microorganism or cell with a candidate compound in the presence of an antimicrobial or cytotoxic agent;
(b) determining whether said microorganism or cell has increased sensitivity to the antimicrobial or cytotoxic agent as compared to a microorganism or cell that is not treated with the candidate compound,
wherein increased sensitivity indicates that the candidate compound enhances the sensitivity of the microorganism or cell to the antimicrobial or cytotoxic agent.

8. A method of identifying a compound that inhibits induction of the SOS response by an antimicrobial or cytotoxic agent, the method comprising:
(a) contacting a microorganism or cell with a candidate compound in the presence of a sublethal dose of an antimicrobial or cytotoxic agent, wherein said micoorganism or cell comprises a polynucleotide encoding a reporter polypeptide, wherein expression of said reporter polypeptide is induced upon activation of the SOS response pathway; and
(b) determining whether expression of the reporter polypeptide is reduced in the microorganism or cell contacted with the candidate compound according to step (a) as compared to a microorganism or cell comprising said polynucleotide that is not treated with the candidate compound,
wherein reduced expression of the reporter gene indicates that the compound inhibits induction of the SOS response by the antimicrobial or cytotoxic agent.

9. The method of claim 7 or 8, wherein said method comprises contacting a plurality of discrete populations of microorganisms or cells with different candidate compounds.

10. A method of identifying an agent that is cytotoxic for a drug-resistant microorganism or cell, the method comprising:
(a) screening one or more candidate agents for their ability to bind or inhibit an activity of a polypeptide associated with double-stranded DNA break repair or stalled replication fork rescue or repair; and
(b) identifying a candidate agent identified in step (a) that is cytotoxic for a drug-resistant microorganism or cell,
thereby identifying an agent that is cytotoxic for a drug-resistant microorganism or cell.

11. The method of claim 10, wherein said microorganism is a gram positive or gram negative bacteria.

12. A method of enhancing the activity of an antimicrobial or cytotoxic compound, comprising administering said antimicrobial or cytotoxic compound in combination with at least one agent that inhibits an activity of a polypeptide associated with double-stranded DNA break repair or stalled replication fork rescue or repair.

13. The method of claim 12, wherein said agent and antimicrobial compound are administered to a subject at risk of, diagnosed with, or suspected of being infected with a microorganism or having a tumor.

14. A method of inhibiting the growth or proliferation of a microorganism or cell, comprising: administering to said microorganism or cell an agent that inhibits an activity of a polypeptide associated with double-stranded DNA break repair or stalled replication fork rescue or repair.

15. The method of claim 14, wherein said microorganism or cell is drug-resistant.

16. The method of claim 14, wherein said agent is administered to a subject at risk of, diagnosed with, or suspected of being infected with a microorganism resistant to one or more antimicrobial compounds or having a tumor.

17. A method of sensitizing a microorganism or cell to an antimicrobial or cytotoxic compound, comprising contacting said microorganism or cell with an agent that inhibits an activity of a polypeptide associated with double-stranded DNA break repair or stalled replication fork rescue or repair.

18. The method of claim 17, wherein said agent is administered to a subject at risk of, diagnosed with, or suspected of being infected with a microorganism resistant to one or more antimicrobial compounds or having a tumor.

19. A method of treating a subject infected with a microorganism, the method comprising:
(a) determining if said microorganism contains a mutation in a gene associated with resistance to an antimicrobial compound; and
(b) administering an agent that inhibits an activity of a polypeptide associated with double-stranded DNA break repair or stalled replication fork rescue or repair to said subject, if said microorganism contains a mutation in a gene associated with resistance to an antimicrobial compound.

20. A method of inhibiting the acquisition of drug resistance by a microorganism, the method comprising: contacting said microorganism with an agent that inhibits an activity of a polypeptide associated with double-stranded DNA break repair or stalled replication fork rescue or repair during treatment with said drug, wherein said agent inhibits transfer of a resistance-conferring gene.

21. A kit comprising an antimicrobial or cytotoxic compound and an agent that inhibits an activity of a polypeptide associated with double-stranded DNA break repair or stalled replication fork rescue or repair.

22. The kit of claim 21, wherein said compound is a fluoroquinolone or a topoisomerase poison.

23. A process of producing a compound that enhances the sensitivity of a microorganism or cell to an antimicrobial or cytotoxic compound, the process comprising:
(a) screening a library of compounds to identify a compound that inhibits an activity of a polypeptide associated with double-stranded DNA break repair or stalled replication fork rescue or repair;
(b) derivatizing the identified compound;
(c) testing the derivatized compound for its ability to inhibit an activity of a polypeptide associated with double-stranded DNA break repair or stalled replication fork rescue or repair; and
(d) producing the derivatized compound,
thereby producing a compound that enhances the sensitivity of a microorganism or cell to an antimicrobial or cytotoxic compound.

24. A composition comprising at least one antimicrobial or cytotoxic compound and at least one agent that inhibits a polypeptide associated with double-stranded DNA break repair or stalled replication fork rescue or repair.

25. The composition of claim 24, wherein said compound is a fluoroquinolone or a topoisomerase poison

26. A method of increasing the therapeutic index of an antimicrobial or cytotoxic compound, the method comprising providing the antimicrobial or cytotoxic compound in combination with an agent that inhibits a polypeptide associated with double-stranded DNA break repair or stalled replication fork rescue or repair.

27. The method of claim 26, wherein said compound is an antibiotic or a chemotherapeutic agent.
